SEQUENCE LISTING (92 sequences)

SEQ ID NO: 1 - protein, 348 aa, Artificial Sequence
  SEQ ID NO. 1 OMT1 (prtn_IY6UUoCV)
MQKWSNGEHSNELLHAQAHIWNHIFSFINSMSLKSAIQLGIPDIINKHGYPMTLSELSSALPIHPTKSHSVYRLMRILVHSGFFAKKKLSKIDEEGYTLTDASQLLLKDHPLSLTPFLTAMLDPVLTKPWNYLSTWFQNDDPTPFDTTHGMTFWDYGNHQPNIAHLFNDAMASDARLVTSVIIDDCKGVFEGLESLVDVGGGTGTVAKAIADAFPHIECTVLDLPHVVGDLQGSKNLKYTGGDMFEAVPPADTVLLKWILHDWNDEECIKILKRSRVAITSKDKKGKVIIIDMMMENQKGDEESIETQLFLDMLMMALVGGKERNEKEWAKLFTDAGFSDYKITPILG

SEQ ID NO: 2 - protein, 368 aa, Artificial Sequence
  SEQ ID NO. 2  OMT2 (prtn_tGvOasTp)
MEADGEDAVLRGQVEIWKYMLSFADSMALKCAVELQLADIIHSHTSPITLSQIASAIPGATSPDLSCLARIMRLLVRRRIFTQHQKPKSDGEEEEALYGPTHSSRWLLTKTNDHDQLTLAPMILMENDPRLMAPWHCFSRCVKEGGVAFKKAHNGQSIWEFGAENPEINKLFNDAMECTAKVVMKAILSHYSDGGFSDIKSMVDVGGGTGGSISEIVRSYPHIKGINFDLPHVIATAPPYSGVSHVGGDMFRSVPTADAIFMKWILHDWSDEDCVKILKNCRKAIAEESGKVIIVESVLEEESNNNNNNNEVFGDTALMLDLVMVAHTTGGKERTQKQWKTILEQGGFPRYNFIKIKALPSIIEAYPN

SEQ ID NO: 3 - protein, 356 aa, Artificial Sequence
  SEQ ID NO. 3  OMT3 (prtn_zYoR6zPg)
MELTTTAARPGLRHRMQQLIYGFFTAQTLHVAVRLRIPDLLADGARDVGDLASATGADAPSLRRLLRALVFLEVLDEPAPGTFALTEQGEVLRADVTGSMRELVLLLSGPESWAAWGQLEHSVRTGEVAWEHVHGRSCFDHLMADPQRQAAFNAAMAEGSRAFVPTLLSAYDFGDLRTVVDVGGGSGALLAGVLAAHPHLRGTVFDTPDGVADAARTVAEQGVADRCGVETGDFFVSVPPGADAYVLKSVLHDWDDEQCVEVLRTVRRAVRPDSRVILVESLMPTTVTTAPSVAQVVMNDLNMMVCHGGRERTVAEFRELLRVAGFRLESVTPCPAPSVVGILEAAPAPATGPDGS

SEQ ID NO: 4 - protein, 370 aa, Artificial Sequence
  SEQ ID NO. 4 OMT4 (prtn_icqpqfVn)
  misc_feature (210)..(210): Xaa can be any naturally occurring amino acid
MASLEEPKGLPDIPLDDEARKEEESYCHALQLVVSSVLSFSMQSAIELGVFDIIAKEGQNAKLSSSEIAAHIGTKTPDGPMMLDRLLAVLASNSVLDCTVVNGKLDKCFRRLYSLTPVSKHFVTNEDGVSLAPVLTMVQDEAFLKGWRQVKDSVIEGGIAFDRAHGMHHFQYPSVDHRFNEIFNKAMFNHSTIVMKRILKLYKGFEHVTXLVDIGGNLGGAISLITSKYPHIKGINFDLPHVIKHASSYPGVENVGGDMFESIPNGDAIFLKFILHDWLDKDCIKLLKNCYNAIPDNGKVIVVEALLPIKPDSNLSVRTNGQLDLHMMTQTPGGMERSQEEFMALATASGFSGIRYECFTANLWIMEFYK

SEQ ID NO: 5 - protein, 409 aa, Artificial Sequence
  SEQ ID NO. 5 OMT5 (prtn_DiPDY0qT)
MISVVSTQRRPSYGGSYSTKCKSLYRLYLYSLNMEKLKSFRHLNNNIDLVLNEENSIELLGAQGHIWNQIFNFINSMSLKCAIQLGIPDIVNNYRKPMTISQLVLALPINQKKSPCVYRLMRILIHSGFFALQKVEGGGEGEEEEGYVITDASKLLLKDNPMSVTPFLLSMLDPVMTKPWDFLSNWFQNDDPTPFDTANGMTFWDYGSHQPNLARFFNDAMASDARLVTSVVIEKCKWVFEGVESLVDVGGGTGTVATSIATNFPQIQCTVLDLPHVVADLQGGNNLNFVGGDMFVEVPTAEVVLLKWILHDWNDEESVKILKKCKEAIMKSKKKGGKVIIIDMKVENEKDEDDESYETQLFFDMLMMTLLTGKERNEKEWAKLFKDAGFSDYKITPILGLRSVIEVYP

SEQ ID NO: 6 - protein, 335 aa, Artificial Sequence
  SEQ ID NO. 6  OMT6 (prtn_4adQLiqx)
MTEETAWGGGLWAAADLITPMAVRVAATLRLGDHITAGSGTPESLAAATGTHPDTLRRVLDHLVTAGLLTRPEPGVYGLTDLGEQLRDDHPGGIRSWLDMTGAIGRAELSFVDLLHTVRTGEAAYPHRYGRPFWEDVAADPDRAASFDALMGSRLRDDAPAVATGYPWGTLGHLVDVGGGDGTLLIAILAAHPGLRGTVLDLPGPAERARAAIDAAGLNDRADARPGSFFDELPPGAGGYLLSGILHDWDDAGATRILRNCAQAAGGRGRVLVMEDDVADEPSTEGDLRMLCYVNGRERGVTQLGDLAAAAGLVVETVVPAGTRAIVEMRRDTGK

SEQ ID NO: 7 - protein, 177 aa, Artificial Sequence
  SEQ ID NO. 7  OMT7 (prtn_qe919VDm)
MTSTWYTLLVLAVAAERLAELVVARRNTRWSLARGGVESGRGHYPAMVALHTGLLLGCLAEVRFAERPFPAVLGWTMAGVVVASQALRWWCIRTLGPRWNTRVIVVPGLPRVTGGPYRWLSHPNYVAVAAEGIALPLVHGAWVTALVFTALNAALMVVRIRCEDGALARLPAADAPA

SEQ ID NO: 8 - protein, 389 aa, Artificial Sequence
  SEQ ID NO. 8  OMT8 (prtn_wQbqeQW9)
MEVMRVLHMNKGNGETSYAKNSTAQSNIISLGRRVMDEALKKLMMSNSEISSIGIADLGCSSGPNSLLSISNIVDTIHNLCPDLDRPVPELRVSLNDLPSNDFNYICASLPEFYDRVNNNKEGLGFGRGGGESCFVSAVPGSFYGRLFPRRSLHFVHSSSSLHWLSQVPCREAEKEDRTITADLENMGKIYISKTSPKSAHKAYALQFQTDFWVFLRSRSEELVPGGRMVLSFLGRRSLDPTTEESCYQWELLAQALMSMAKEGIIEEEKIDAFNAPYYAASSEELKMVIEKEGSFSIDRLEISPIDWEGGSISEESYDLVIRSKPEALASGRRVSNTIRAVVEPMLEPTFGENVMDELFERYAKIVGEYFYVSSPRYAIVILSLVRAG

SEQ ID NO: 9 - protein, 398 aa, Artificial Sequence
  SEQ ID NO. 9  OMT9 (prtn_9dxmAvGq)
MSAASPASIIQELASAAKQYENNESGAREALIAQSRALIASLEVPSEFIQHTFWSQPALSAIVRLATDVNLFQYLKDAQEEGLSAEALASKTGMDVSLFARLARHLVAMNVITSRNGVFYGTALSNGLAAENYQQSIRFCHDVSRPSFGAFPSFFKGNGYKTPALGTTDGPFQSAHKVDISFPQWLVGNPPYLQYFNSYMSAYRAGKPNWCDNGFYPVADRLLNGFDASVSDVLLVDVGGGRGHDIATFGSQFSPLPGRLVLQDREQVINSIPADESRQFEATTHDIFTTQPVKNARAYYMHSVPHGFGDEDAVKIMANLVPALAKGYSRVLLNEIVVDEESPVMSATNMDLIMLAHMGAKERTEADWRSILTRAGLKVVNIYSYPGVAESLIEAELA

SEQ ID NO: 10 - protein, 366 aa, Artificial Sequence
  SEQ ID NO. 10  OMT10 (prtn_4cW1vabL)
MALPNGISSKQELLEAQAHVWNHIYSYINSMSLKCAIQLGIPDAIHKHGNPITLSQLADALNINKAKSHGLFRLMRILVHSGFFDKVKVKVKVEGEDEEEEEDAYSLTPASRLLLRSEPLSVAPFALAMSDPVYTETWHHLSEWFRNDAVAAFDTKYGMTFPEYAVADDRLNVLFNEAMACDAGFVNSILTTECREIFDGLESMVDVGGGTGATAKGIAAAFPGMECTVLDLPNVVGGLKGSENLSFVSGDMFDFIPHADAIFMKFILHDWNDEECVKILKKCKEAISRSNNSCRKIILVEIVMEDEKETHEATETKLFFDMQMLAIITGKERSEKEWGKLFFDAGFTNYKITRVLGLRSVIEVFP

SEQ ID NO: 11 - protein, 312 aa, Artificial Sequence
  SEQ ID NO. 11   OMT11 (prtn_pa63UK5p)
MLLRSRFLKVIHVRKQLSACSRFAIQTQTRCKSTDASEDEVKHFQELAPTWWDTDGSQRILHKMNLTRLDFVQRTVRNQVKIQNPEIFVPGFNYKEFLPEYVCDNIQREMQESIETNLDKRPEVSVLDVGCGGGILSESLARLKWVKNVQGIDLTRDCIMVAKEHAKKDPMLEGKINYECKALEDVTGQFDIITCMEMLEHVDMPSEILRHCWSRLNPEKGILFLSTINRDLISWFTTIFMGENVLKIVPKGTHHLSKYINSKEILAWFNDNYSGQFRLLDLKGTMYLPYQGWVEHDCSDVGNYFMAIQRLN

SEQ ID NO: 12 - protein, 365 aa, Artificial Sequence
  SEQ ID NO. 12   OMT12 (prtn_JoZt2PLE)
MGSTGETQITPTHISDEEANLFAMQLASASVLPMILKSALELDLLEIIAKAGPGAQISPIEIASQLPTTNPDAPVMLDRMLRLLACYIILTCSVRTQQDGKVQRLYGLATVAKYLVKNEDGVSISALNLMNQDKVLMESWYHLKDAVLDGGIPFNKAYGMTAFEYHGTDPRFNKVFNKGMSDHSTITMKKILETYTGFEGLKSLVDVGGGTGAVINTIVSKYPTIKGINFDLPHVIEDAPSYPGVEHVGGDMFVSIPKADAVFMKWICHDWSDEHCLKFLKNCYEALPDNGKVIVAECILPVAPDSSLATKGVVHIDVIMLAHNPGGKERTQKEFEDLAKGAGFQGFKVHCNAFNTYIMEFLKKV

SEQ ID NO: 13 - protein, 374 aa, Artificial Sequence
  SEQ ID NO. 13  OMT13 (prtn_1QwPODPY)
MVLISEDSRELLQAHVELWNQTYSFMKSVALAVALDLHIADAIHRRGGAATLSQILGEIGVRPCKLPGLHRIMRVLTVSGTFTIVQPSAETMSSESDGREPVYKLTTASSLLVSSESSATASLSPMLNHVLSPFRDSPLSMGLTAWFRHDEDEQAPGMCPFTLMYGTTLWEVCRRDDAINALFNNAMAADSNFLMQILLKEFSEVFLGIDSLVDVAGGVGGATMAIAAAFPCLKCTVLDLPHVVAKAPSSSIGNVQFVGGDMFESIPPANVVLLKWILHDWSNDECIKILKNCKQAIPSRDAGGKIIIIDVVVGSDSSDTKLLETQVIYDLHLMKIGGVERDEQEWKKIFLEAGFKDYKIMPILGLRSIIELYP

SEQ ID NO: 14 - protein, 365 aa, Artificial Sequence
  SEQ ID NO. 14  OMT14 (prtn_u5QQ2uY8)
MGSTVETQMTPIQVSDEEANLFAMQLASGSILPMVLKAAIELDLLEVMAKAGPGAFVSPADLASQLPTKNPDAPVMLDRMLRLLASYSILTYSLRTLPDGKVERLYGLGPVCKFLTKNEDGASIGSLCLMNQDKVLMESWYHLKDAVLEGGIPFNKAYGMTAFEYHGTDPRFNKVFNKGMADHSTITMKKLLEIYNGFEGLTSIVDVGGGTGAVLNMIVSKYPSIKGINFDLPHVIEDAPQYPGVEHVGGDMFVSVPKGDAIFMKWICHDWSDEHCLKFLKNCYAALPDNGKVIVAECILPVAPDSSLATKGVVHIDAIMLAHNPGGKERTEKEFEALAKESGFQGFRVVCSAFNIYAIEFLKKI

SEQ ID NO: 15 - protein, 365 aa, Artificial Sequence
  SEQ ID NO. 15   OMT15 (prtn_MOfy0Tz9)
MGSTGETQMTPTQVSDEEANLFAMQLASASVLPMVLKAAIELDLLEIMAKAGPGVFLSPTDIASQLPTKNPDAPVMLDRMLRLLASYSILTYSLRTLADGKVERLYGLGPVCKFLTKNEEGVSIAPLCLMNQDKVLVESWYHLKDAVLEGGIPFNKAYGMTAFEYHGTDPRFNKVFNRGMADHSTITMKKILETYKGFEGLTSVVDVGGGTGAVLNMIVSKYPSIKGINFDLPHVIEDAPQYPGVEHVGGDMFVSVPKGDAIFMKWICHDWSDEHCLKFLKNCYAALPDNGKVILGECILPVAPDSSLATKGVVHIDVIMLAHNPGGKERTEQEFQALAKGAGFQGFNVACSAFNTYVIEFLKKN

SEQ ID NO: 16 - protein, 365 aa, Artificial Sequence
  SEQ ID NO. 16  OMT16 (prtn_uh3ufF5t)
MGSTPETQMTPTQVSDEEANLFAMQLASASVLPMVLKAAIELDLLEIMAKAGPGAFVSPADLSSQLPTKNPDAPVMLDRMLRVLASYSILTYSLRTLPDGKVERLYGLGPVCKFLTKSEDGASIGSLCLMNQDKVLIESWYNLKDAFLEGGIPFNKAYGMTAFEYHGTDPRFNKVFNKGMADHSTITMKKLLENYNGFEGLTSIVDVGGGTGAVLNMIVSKYPSIKGINFDLPHVIEDAPQYPGVEHVGGDMFVSVPKGDAIFMKWICHDWSDEHCLKFLKNCYAALPDNGKVIVAECILPVAPDSSLATKGVVHIDAIMLAHNPGGKERTEKEFEALAKGSGFQGFRVVCSAFNTYAIEFLKKN

SEQ ID NO: 17 - protein, 352 aa, Artificial Sequence
  SEQ ID NO. 17  OMT17 (prtn_cwrlQpnu)
  misc_feature (311)..(311): Xaa can be any naturally occurring amino acid
MESLRGQEQIWQLMFSFVDSMALKCAIELRIADIIHSHGKPITLSQIASGIRSNSNSSISPNIPYLSRIMRFLVRKNIFTEHQEDNDEVISLYGLSDSSRWLLRDFKSSLAPMVLMQTHPLSMAVWHFLEDYVRNSSNTFEKAHGCNIWEFSSANPDFNKIFNNAMASIVPIYMGAVLSSYKDGLGCIKGTVVDVGGGTGGSISELMKYYPNIKGINFDLPHVIATAPALDGVTHISGDIFESIPSADAVLMKGVLHCFSDEKCVKVLRNCRKAITDKKNGKIIILEIVLDPTSNQIFDETRMVYDLLIPXFSGGKERTELEWKRLLNEAGFTSIKITKIPIIPAIIEAFLV

SEQ ID NO: 18 - protein, 360 aa, Artificial Sequence
  SEQ ID NO. 18  OMT18 (prtn_8NkMs4lT)
MELARNDQTEAALRGEANVWKSINGIADFMVMKCALELRIPDIVHSHSAPITLAQIASSVPDSPSLNLSYLSRIMRLLVRRKIFSQHKSLDGEEVLYGPTHSSRLLLSKTTLPDQVTLAPFVAFMTHPYLSAPWSCLARCVKEGGNGFEMVHGGRQLWDLSPGNPEFNKVFNDGMASTARITTMAILSEYRDVFCGICSLVDVGGEFGGSISAIVKSHPHIKGINYDLPHVVATAPTYTGLVSHVGGNMFEWIPTAVAVFMKWILHDWADEDCVKILKNCRRAMPEKGGKIIIVDIVLEPEGNGLFDDAAVMLDIALMALTRGKERTEKEWKRVLEEGGFPRYQILKIPALTSVIEAYPQ

SEQ ID NO: 19 - protein, 439 aa, Artificial Sequence
  SEQ ID NO. 19  OMT19 (prtn_7wFbK88Q)
MGSISSPSLIIDLANAVSSAAKNLDTQLQSQGLPQPSFEADGPTYVVPKDAPKAAHEARVATAEAALKLFNLVSGPSELLPNMTASYHTIFALQWLHHFDVFSHIPLDGTLSYEKLATKANVPESLLKSVARMAMTSNILAEPTTGQVAHSASSAMFVKFPNMRDWASYMFTASIPTAAAMVQATEKWPGSVKKTETAYNIAFNHDLPFFDHLSQSPVMTKQFSGYMRSVTDGQGMDLSHLVNGFDWASLPDKSLIVDIGGSAGHASYALAAAYPHLRFEVQDLDTVVNGEKAAKEHEEAVSKHVLGTDNRVTFKAHNFFEAQPTKDATVYMLRMIIHDWPDAEAKTILGNLVPALESAKATLLIMDTVLPSPGSIPSVRERVIRTRDLTMRQVFNAKERGVDDWEAILRETDSRLTLKNLRQPEGSNMCLLTISLQDD

SEQ ID NO: 20 - protein, 378 aa, Artificial Sequence
  SEQ ID NO. 20  OMT20 (prtn_RBrV7WR9)
MTSDQPAATAVENPLPERDEPMTSEATLARFREYMVGPSRFMTLLSCFELGLVDQIRDNPGLTAAELGEAIGAKADAVEQLLLLLVKEGFVAHDEASGAYVLDGLADVAAGDLKRALAYMNMIKVVALRQLFHLTESAQTGTLVGLKELYGVTEGTLYGAVAEHRDLRDAWSNLMNTVTANIDPWFFGNVDVPAGARVLDLAGNTGLGAIHTVAHKASPGLQVTTFDLPEKEQEALANFKAHGVAESCSFIGGDVFDGVPKGFDIVLIKHFLDMFDKDDVIRILQGVNQALEVGGQVNIMVPVYPEDITDTDNYNVDFFPAFFIGCTMGQGGPQKLSAYQSWLEECGFKVTKAITKNAAEVPPDVIPVQAIISATKVV

SEQ ID NO: 21 - protein, 427 aa, Artificial Sequence
  SEQ ID NO. 21  OMT21 (prtn_HlzCGwyK)
MALKSTNGTHAGPTASAASLASLAANISEKAASLSTYLESQGHAQPSFLPGCADPPETEEYLALHTSLTSSLEDLQRLVDGPRRSLRPFIMIGNDLAALQVAFDFGFFQLIPPEGSMDVETLAHKVGIDADRTARVLRMLATHRIFVEPKPGFFAHTAASAVFHDDEELRCAGHYMLDECFKAATACSDCIKASPNDSDSTHSPFNTYFGVPMFSYYEQNPQFAARFAKAMAVDRQIAELRDCFPWGDIKGTVVDVGGGSGHISMALARNFPKLDFIVQDDSEKMLAQGRARNLSDIEGRISFMKHSFFHPQPIGGAGAFFIRQCTHNWCDRDVVKILKSFVPGLENSAPGTPLLINDTVLPVPGSKPLHEERALRQMDMLMFVVLGAKQRTAKEFEALLKEADARYEIRRVHADGSMGLVEVHLNI

SEQ ID NO: 22 - protein, 378 aa, Artificial Sequence
  SEQ ID NO. 22  OMT22 (prtn_Dse0xKlS)
MSEHQPTHATTPTHPAAGAQSGPDTGPAAGAQSGPDTGPAAGAQSGPDAGPDTGPDTVLRLINGYWSTGILGAAAEHQLFTHLEAGAHDAAQLAQRAQISERGAQTLLDGLVSIGLLTLEQGRYRNTPAASAYLVTGRPADLSQMARLKLTHMGKLTALPEVVRAGGPVSDPTTEVADNPHWGQVVPAIAAQSVPAAAVAADVLGLAGAGAISILDVGGGSGIYSATWLEANPAARATQLDWEPINAIARKLLAERGVGDRFTCIDGDFHTTDFGTGRYDVALYSHIAHQEGPEDNIAVFTRLKDALKPGGALVVCDYVVDDDRSGPAFPLLFASEMLLKSQQGGTWRRADYHAWLTKAGFSDVSFHAAPPATLVIAR

SEQ ID NO: 23 - protein, 344 aa, Artificial Sequence
  SEQ ID NO. 23  OMT23 (prtn_Y15oO7Wp)
MVGPSRFMSLLSCFELGVIDKLRDTPGMTAAELGDAVGAKPDAVEQLLLLLVKESFLAYDEDTGGYSLDALADIAEDDLRQVLARMEMIKVVTLRQLFYLTDSVRTGTTVGLNKLYGHEGNLYDALAEHKDLREPWARLMNGETAHIDPWFFDNIDVPPGSNVLDLAGNTGLGAIHTCKLKASPGLRVTTFDLPEKEEECLANFRSHGVEEHCSFIGGDVFEEVPKGFDVVLIKHFLPMFDKSDVFKILEGVNRSMDVGGQVHLLVPVFPENIKDSDNYTVDFFPSFFIGCAMGQGGAQKMSTWQKWLEECGFTVTKAIAEDPADMLPHALPVEAVLSATKRAG

SEQ ID NO: 24 - protein, 429 aa, Artificial Sequence
  SEQ ID NO. 24  OMT24 (prtn_hFdRi4bZ)
MADQANTAVQGSSPSILALAQNILELTQDVTKYFQANNLAAPTFALGSQDPPNTPEYRRLHATLKTSLEDLQRLIDGPRKWLRGFCCTGYDLGALQIALDFNFFQLIPPHGDITFEDLAQKAGLDLDRTSRVVRQLMTYRFFEELRPRVISHSSTSLLMQQDEELRSVVHYSLDEMLKAAADCNISLKANPYEAHQNLNPFVTRHGVGIFEFYKNDSEKARRFAKAMAGLRRMDRHLDYLLTDGFNWSAVKGTVVDCGGGNGHISKSLAHLYSDLDFVVQDSNAEMLAEGQDSLHDDLRGRVTYLQHSFFEPQPTKGAAAFLIRQCTHNWADHDVVTIFKSFVPGLEHSSPDTPLLINDIIIPEPGAWPRHQERAMRQVDMIMLVNCGAKQRTKAEFELLLKEADPRYEIRNVFDNGPLGLLEVYLNRP

SEQ ID NO: 25 - protein, 435 aa, Artificial Sequence
  SEQ ID NO. 25   OMT25 (prtn_3bVmhBhw)
MAAQHDHLKSTNDDPAGRLSTNSLLNLAELILQQTTSINGYLRENAIAPPTFDADSSKPPETAEYQALHGSLTSSLEDLQRLIDGPKESIRSYLLVGFELAAAQVAFEFDFFNIIPEDGGMEVGELAKKAGMDADRAARFLRILATRRIFQEKTPGHFSHTALSRAFAKDEDIKCTGQFATDELLKAATSSADCVKASPYESDSAHCPFSTRHGMSMFEFYGKNPRYAARFAKAMAGITKIDRQITELKDCFPWENLQGTVVDVGGGSGHTSISLARKFPHLSFVVQDGSADMLAQGKKLDLHDVEGQVTWMQYDFFQPQPLRDVAAFFIRQCTHNWCDRDVVKIFKSFVPGLEGSKPGTPLLINETILPEPGTKPLHEERAFRQMDMLMYVNLGAKQRTKNEFEALLKEADPRYEIHNVHAEGNMGLLEVHLRR

SEQ ID NO: 26 - protein, 395 aa, Artificial Sequence
  SEQ ID NO. 26   OMT26 (prtn_q1ERTwS4)
MTSVPATNGAEQRSAGKDVSMIGLAADIARQATRIHKYLQGTEARDLSEGGGSIDPPETAEYALLRNQLTSSLEDLQHLIDGPRTYLRSFICSGNDLAALQVGFDFNFFTIVPEEGDIGISELADKAGLDVDRASRVVRMMITRRLFQEAIPGRIAHTTGSRIFKTDENLRCAGQYMLDEMWKAATASSDCIKASPHESDIDRQISELQDNYPWADLKGTVVDVGGGSGHVSVALARLFPHLSFTVQDASPHMIAQGQSLDLSGVEGRVFWMQHDFFQPQPVRDAGAFVVRQCLHNWCDRDVITIFKSLVPGLEGSAPGTPLLINDTLLPEPNKIPAHVERSMRQMDMLMFVAVGSKERTKDDFEMLLKEADPRFEIHRVLAEGSMGLIEVHLRR

SEQ ID NO: 27 - protein, 422 aa, Artificial Sequence
  SEQ ID NO. 27  OMT27 (prtn_S7nwBKtt)
MSLPVQTPIQLVALAESILQQTKNISQYLKSHAHSEMTFDSVYTEPPNDPEFVQLQDSLKTSLEDLRFLADGPTRFYRSFFMTGYLLAAFQLALDLAFFDLVPPDGAISFQELAGKSGLDADRTKRVVCYLATHHVFRISQSGLVSHTAFSIAMRDEELRSVVHYSFDEMLKAAVETGSSLKATPQESDKTHCPFTSRHGVSIFEYYAKHTDKALRFAKAMAAYRRMENSTVELRDNFPWQRLEGTVVDIGGGSGHISMALAREFPNLKFIVQDDSEDMLSDGQRLLTPDIQDRIVFQKQSFFAPQPCRDIAAFVIRQCTHNWADDDVIVMFKSVVPGLEGSSPDTPLLINDIVMPEVGTVPRLKERAMREADMVMMVSFGAKQRTKAEFDILLKKADPRFEIRKVHDTGALGLLEVYLHRS

SEQ ID NO: 28 - protein, 423 aa, Artificial Sequence
  SEQ ID NO. 28  OMT28  (prtn_8BtPlxhk)
MPSQSNGAHGSGLLAVAEDIVQQTQRIVKYLNANNIEQPTFATDDTIRPETLDYAKLQGELKRSLEDLQYLVEGPKRHFRNLACQGYELAAISVALDFNFFSIVPAEGKISLEELAERAGVDLDRTTRIVRLLATESIFREPTPGYVAHNPSSYLLHKDEEIRATLHYTLDEMFKAATATADNVKASPKSYDSAVTPFTTRHGLPIFNFYEKDTQRSIRFAKAMAGWAKLNLNIDALKDSFPWGDLDGTVVDVGGGSGKVSITLAKSFPNLKFIVQDSADMHVAGQSLLTDDTRGRVTFMQHSFFDPQPVGDAAAFILRACALNWCDARVVDMLRALVPGLKRSKPGTPLLINDMILPEQGVLTRDVERGMRQIDLIMLVGFGGKLRTADEFEALLKQADERYEIRSVRPEGLMGLIEAHLRR

SEQ ID NO: 29 - protein, 374 aa, Artificial Sequence
  SEQ ID NO. 29   OMT29 (prtn_VXdQd8Af)
MAMANGSSSSLSASWSSAELFKAQGQVWNCALNCVNSLSLKCAVELGIPDIIHSHGQPMTLSQICSALNIQPNKTQAMERVMRILVHSGFFSLSRDDDPQGYYGLTPASRLLLKDEPFGAAKFLLFMTHPHMTDSLNSLSSWFQNDVPTSFETTHGKGYWEFVAGEDKFSKIFYDCMEADSELIVSVLIKDYKEVFEGIGSLVDVGGGTGATALALAKAFPEINFTVFDQPHVVHNLQGTQNLRFQGGDMFDAIPPADAFLLKWTSHDWGDEDCVKILKKCKEAIPSKGKVIIIEIAIKENSKEEAEEEKEKDDCSSKTETETQLCFDMMCMQAYNFGKQRTVREWEKVLEGAGFSHYKITPCLGARSLIEAYP

SEQ ID NO: 30 - protein, 224 aa, Artificial Sequence
  SEQ ID NO. 30   OMT30 (prtn_w2T6OUA7)
MSESQQLWDDVDDYFTTLLAPEDEALTAALRDSDAAGLPHINVAPNQGKLLQLLAEIQGARRILEIGTLGGYSTIWLGRALPRDGRLISFEYDAKHAEVARRNLARAGLDGISEVRVGPALESLPKLADERPEPFDLVFIDADKVNNPHYVEWALKLTRPGSLIVVDNVVRGGGVTDAGSTDPSVRGTRSALELIAEHPKLSGTAVQTVGSKGYDGFALARVLP

SEQ ID NO: 31 - protein, 458 aa, Artificial Sequence
  SEQ ID NO. 31  GLY1 (prtn_aZpQN5Ei)
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 32 - protein, 462 aa, Artificial Sequence
  SEQ ID NO. 32   GLY2 (prtn_aKbna2uv)
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 33 - protein, 340 aa, Artificial Sequence
  SEQ ID NO. 33  GLY3 (prtn_6Pfe14RE)
MISVIIPLYNQERYIQDCLASLAAQTMDDFEVIIVDDGSTDSSAELAADFAAQDSRFTLMRQANAGVSTARNRGLDQATGQWVCFVDPDDSVDKDYLATLLAASAEQQDIDIVMSTCVALTDDGEVRQHFFPESFVAHTAQDKERLFHQLIDGAFEQSDGFVTAIGVPWGKLYRRDFLAEHDLRFDPALPRMQDNLFNMEAFQHARAITYLDYAGYRYRVGGLSERTYRNTIKGLYHPAIDRRAELMHAYGLDQVPQLYQAWQVEQVNLYYQELKAAAMLTDGSAAQVARVVRARADTLRQRTAQVDAAVLPRPIRLKYRMLIAPAMTTLAALALARQRH

SEQ ID NO: 34 - protein, 470 aa, Artificial Sequence
  SEQ ID NO. 34   GLY4 (prtn_4BalFvlK)
MVQPHVLLVTFPAQGHINPCLQFAKRLIRMGIEVTFATSVFAHRRMAKTTTSTLSKGLNFAAFSDGYDDGFKADEHDSQHYMSEIKSRGSKTLKDIILKSSDEGRPVTSLVYSLLLPWAAKVAREFHIPCALLWIQPATVLDIYYYYFNGYEDAIKGSTNDPNWCIQLPRLPLLKSQDLPSFLLSSSNEEKYSFALPTFKEQLDTLDVEENPKVLVNTFDALEPKELKAIEKYNLIGIGPLIPSTFLDGKDPLDSSFGGDLFQKSNDYIEWLNSKANSSVVYISFGSLLNLSKNQKEEIAKGLIEIKKPFLWVIRDQENGKGDEKEEKLSCMMELEKQGKIVPWCSQLEVLTHPSIGCFVSHCGWNSTLESLSSGVSVVAFPHWTDQGTNAKLIEDVWKTGVRLKKNEDGVVESEEIKRCIEMVMDGGEKGEEMRRNAQKWKELAREAVKEGGSSEMNLKAFVQEVGKGC

SEQ ID NO: 35 - protein, 482 aa, Artificial Sequence
  SEQ ID NO. 35   GLY5 (prtn_IKVHSLEK)
MKETKKIELVFIPSPGIGHLVSTVEMAKLLIAREEQLSITVLIIQWPNDKKLDSYIQSVANFSSRLKFIRLPQDDSIMQLLKSNIFTTFIASHKPAVRDAVADILKSESNNTLAGIVIDLFCTSMIDVANEFELPTYVFYTSGAATLGLHYHIQNLRDEFNKDITKYKDEPEEKLSIATYLNPFPAKCLPSVALDKEGGSTMFLDLAKRFRETKGIMINTFLELESYALNSLSRDKNLPPIYPVGPVLNLNNVEGDNLGSSDQNTMKWLDDQPASSVVFLCFGSGGSFEKHQVKEIAYALESSGCRFLWSLRRPPTEDARFPSNYENLEEILPEGFLERTKGIGKVIGWAPQLAILSHKSTGGFVSHCGWNSTLESTYFGVPIATWPMYAEQQANAFQLVKDLRMGVEIKMDYRKDMKVMGKEVIVKAEEIEKAIREIMDSESEIRVKVKEMKEKSRAAQMEGGSSYTSIGGFIQIIMENSQ

SEQ ID NO: 36 - protein, 478 aa, Artificial Sequence
  SEQ ID NO. 36   GLY6 (prtn_9nh2HTiF)
MKTTELVFIPAPGMGHLVPTVEVAKQLVDRDEQLSITVLIMTLPLETNIPSYTKSLSSDYSSRITLLQLSQPETSVSMSSFNAINFFEYISSYKDRVKDAVNETFSSSSSVKLKGFVIDMFCTAMIDVANEFGIPSYVFYTSNAAMLGLQLHFQSLSIEYSPKVHNYLDPESEVAISTYINPIPVKCLPGIILDNDKSGTMFVNHARRFRETKGIMVNTFAELESHALKALSDDEKIPPIYPVGPILNLGDGNEDHNQEYDMIMKWLDEQPHSSVVFLCFGSKGSFEEDQVKEIANALERSGNRFLWSLRRPPPKDTLQFPSEFENPEEVLPVGFFQRTKGRGKVIGWAPQLAILSHPAVGGFVSHCGWNSTLESVRSGVPIATWPLYAEQQSNAFQLVKDLGMAVEIKMDYREDFNKTNPPLVKAEEIEDGIRKLMDSENKIRAKVMEMKDKSRAALLEGGSSYVALGHFVETVMKN

SEQ ID NO: 37 - protein, 479 aa, Artificial Sequence
  SEQ ID NO. 37   GLY7 (prtn_JZcGfS0m)
MKTAELVFIPAPGMGHLVPTVEVAKQLVDRHEQLSITVLIMTIPLETNIPSYTKSLSSDYSSRITLLPLSQPETSVTMSSFNAINFFEYISSYKGRVKDAVSETSFSSSNSVKLAGFVIDMFCTAMIDVANEFGIPSYVFYTSSAAMLGLQLHFQSLSIECSPKVHNYVEPESEVLISTYMNPVPVKCLPGIILVNDESSTMFVNHARRFRETKGIMVNTFTELESHALKALSDDEKIPPIYPVGPILNLENGNEDHNQEYDAIMKWLDEKPNSSVVFLCFGSKGSFEEDQVKEIANALESSGYHFLWSLRRPPPKDKLQFPSEFENPEEVLPEGFFQRTKGRGKVIGWAPQLAILSHPSVGGFVSHCGWNSTLESVRSGVPIATWPLYAEQQSNAFQLVKDLGMAVEIKMDYREDFNTRNPPLVKAEEIEDGIRKLMDSENKIRAKVTEMKDKSRAALLEGGSSYVALGHFVETVMKN

SEQ ID NO: 38 - protein, 458 aa, Artificial Sequence; identical to SEQ ID NO: 31
  SEQ ID NO. 38   GLY8 (prtn_yAjjYS8A)

SEQ ID NO: 39 - protein, 485 aa, Artificial Sequence
  SEQ ID NO. 39   GLY9 (prtn_0QHKLMIq)
MGSIGAELTKPHAVCIPYPAQGHINPMLKLAKILHHKGFHITFVNTEFNHRRLLKSRGPDSLKGLSSFRFETIPDGLPPCEADATQDIPSLCESTTNTCLAPFRDLLAKLNDTNTSNVPPVSCIVSDGVMSFTLAAAQELGVPEVLFWTTSACGFLGYMHYCKVIEKGYAPLKDASDLTNGYLETTLDFIPGMKDVRLRDLPSFLRTTNPDEFMIKFVLQETERARKASAIILNTFETLEAEVLESLRNLLPPVYPIGPLHFLVKHVDDENLKGLRSSLWKEEPECIQWLDTKEPNSVVYVNFGSITVMTPNQLIEFAWGLANSQQTFLWIIRPDIVSGDASILPPEFVEETKNRGMLASWCSQEEVLSHPAIVGFLTHSGWNSTLESISSGVPMICWPFFAEQQTNCWFSVTKWDVGMEIDSDVKRDEVESLVRELMVGGKGKKMKKKAMEWKELAEASAKEHSGSSYVNIEKLVNDILLSSKH

SEQ ID NO: 40 - protein, 485 aa, Artificial Sequence
  SEQ ID NO. 40   GLY10 (prtn_6rczPyEk)
MGSIGAEFTKPHAVCIPYPAQGHINPMLKLAKILHHKGFHITFVNTEFNHRRLLKSRGPDSLKGLSSFRFETIPDGLPPCDADATQDIPSLCESTTNTCLGPFRDLLAKLNDTNTSNVPPVSCIISDGVMSFTLAAAQELGVPEVLFWTTSACGFLGYMHYYKVIEKGYAPLKDASDLTNGYLETTLDFIPCMKDVRLRDLPSFLRTTNPDEFMIKFVLQETERARKASAIILNTYETLEAEVLESLRNLLPPVYPIGPLHFLVKHVDDENLKGLRSSLWKEEPECIQWLDTKEPNSVVYVNFGSITVMTPNQLIEFAWGLANSQQSFLWIIRPDIVSGDASILPPEFVEETKKRGMLASWCSQEEVLSHPAIGGFLTHSGWNSTLESISSGVPMICWPFFAEQQTNCWFSVTKWDVGMEIDCDVKRDEVESLVRELMVGGKGKKMKKKAMEWKELAEASAKEHSGSSYVNIEKVVNDILLSSKH

SEQ ID NO: 41 - protein, 496 aa, Artificial Sequence
  SEQ ID NO. 41  GLY11 (prtn_tiKIbLIM)
MATQVHKLHFILFPLMAPGHMIPMIDIAKLLANRGVITTIITTPVNANRFSSTITRAIKSGLRIQILTLKFPSVEVGLPEGCENIDMLPSLDLASKFFAAISMLKQQVENLLEGINPSPSCVISDMGFPWTTQIAQNFNIPRIVFHGTCCFSLLCSYKILSSNILENITSDSEYFVVPDLPDRVELTKAQVSGSTKNTTSVSSSVLKEVTEQIRLAEESSYGVIVNSFEELEQVYEKEYRKARGKKVWCVGPVSLCNKEIEDLVTRGNKTAIDNQDCLKWLDNFETESVVYASLGSLSRLTLLQMVELGLGLEESNRPFVWVLGGGDKLNDLEKWILENGFEQRIKERGVLIRGWAPQVLILSHPAIGGVLTHCGWNSTLEGISAGLPMVTWPLFAEQFCNEKLVVQVLKIGVSLGVKVPVKWGDEENVGVLVKKDDVKKALDKLMDEGEEGQVRRTKAKELGELAKKAFGEGGSSYVNLTSLIEDIIEQQNHKEK

SEQ ID NO: 42 - protein, 420 aa, Artificial Sequence
  SEQ ID NO. 42   HAL1 (prtn_l22mfyYU)
MSHPEPEYDVIVVGGGPAGSSTAGLLAQEGHRVLLLEREKFPRYHIGESLISGVTLTLDALGVRERMAELRFQIKHGGSLLWGADQTAPWSFRFREIRDARFDYSWQVRRAEFDAMLLDRARELGVVVVEGATVRGPLTDGERVAGVSYQFRGEADPIDARAAIVVDASGQQRWLGRHFGLVSWHDDLRNMAAWSYYAGALRYPGDHEGDLLVESCAQGWLWYAPLSPTLTGIGYVTPSDRFAETGLPPDQLLEKQIAESNEVSWLTAGARRVDVYRTARDWSYACSQFSGPGWVLVGDAAAFIDPLLSSGVTLAMRGALSLSRAVHEALAAPEKERHLMQVYEDRYRDFLAALLDLIRFFYDGAHGRDELHLRAQAIVDPDRLMPPKISFVSLLSGLARGDETLDRSPRTAIDRPSDAI

SEQ ID NO: 43 - protein, 333 aa, Artificial Sequence
  SEQ ID NO. 43   HAL2 (prtn_bYWHpOol)
MPATRTPIHPEAAAYKNPRYQSGPLSVIPKSFVPYCELMRLELPHGNFLGYFPHLVGLLYGSSASPARLPANEVAFQAVLYIGWTFFMRGAGCAWNDVVDQDFDRKTTRCRVRPVARGAVSTTSANIFGFAMVALAFACISPLPAECQRLGLMTTVLSIIYPFCKRVTNFAQVILGMTLAINFILAAYGAGLPAIEAPYTVPTICVTTAITLLVVFYDVVYARQDTADDLKSGVKGMAVLFRNYVEILLTSITLVIAGLIATTGVLVDNGPYFFVFSVAGLLAALLAMIGGIRYRIFHTWNSYSGWFYALAIFNLLGGYLIEYLDQVPMLNKA

SEQ ID NO: 44 - protein, 519 aa, Artificial Sequence
  SEQ ID NO. 44   HAL3 (prtn_QV8NACx7)
MSVPKSCTILVAGGGPAGSYAAAALAREGNDVVLLEADQHPRYHIGESMLPSLRPLLRFIDLEDKFDAHGFQKKLGAAFKLTSKREGSHGPRGYSWNVVRSESDEILFNHARESGAQAFQGIKINAIEFEPYEEEYPNGEKVANPGKPTSAKWSSKDGSSGDIAFKYLVDATGRIGIMSTKYLKNRHYNEGLKNLAIWGYYKNNIPWAQGTPRENQPFFEGMRDGAGWCWTIPLHNGTVSVGAVMRKDLFFEKKKSLGENATNTQIMAECMKLCPTIGELLAPAELVSDIKQATDYSYSATAYAGPNFRIVGDAGCFIDPFFSSGHHLAVAGALAAAVSINASIKGDCTEYEASRWHAKKVDEGYTLFLLVVMAALKQIRMQENPVLSDVDEDGFDRAFQFLRPEAVKKFTKEDVAQTIDFAVHALNNMAELDMDIPEHMINGDKEGENGVTNGNNGAAKTAGLASNMEKLTNDEEKVLNGLRILGKAAPGGTLADFEGTAIDGLMPRLEHGKLGLNKV

SEQ ID NO: 45 - protein, 530 aa, Artificial Sequence
  SEQ ID NO. 45   HAL4 (prtn_Sb1nup6H)
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 46 - protein, 530 aa, Artificial Sequence; identical to SEQ ID NO: 45
  SEQ ID NO. 46  HAL5 (prtn_KHXKeTwd)

SEQ ID NO: 47 - protein, 409 aa, Artificial Sequence
  SEQ ID NO. 47   HAL6 (prtn_e9NAUpNo)
MSATEEDYDVVVIGGGPGGAATAGLLAGMGHKVLVLEREKFPRYHIGESLITGALPTLDDLGLIKRLDAMGFTRKYGGTLLWGKNQGTWGFRFTESALYDYAYQVRRADFDALVLGRARELGAKVLEEVTVREPLFDGERMVGVSYAEKGSKETKEAHAKFVIDASGQTHMLAKHFDSVVWHDDLRNIATWTYWQGCNLYSGTKAGDIISENRPTGWFWFIPLHDGTVSVGYVTPLEEYQKSGKSLEELYFEELGKTEEVSTLVKGATRASGFRNIKDWSYTAERFHGPGWALVGDAAAFVDPLLSTGVTLALRGARALADAIDGVLHDPSVEDELLGLYEESYRTFLDSVLDFVRFFYDRTKNKEDYWNKAQEQIDPQKLRPRETDFATMLSGLTGINDIFQQRRRAA

SEQ ID NO: 48 - protein, 518 aa, Artificial Sequence
  SEQ ID NO. 48  HAL7  (prtn_SR4isLxL)
MAVPEKCTVLVIGGGPAGSYAASALAREGVDTVLLEADKFPRYHIGESMLASMRHFLRFIDVDSVFDSYGFTKKVGAAFKLNPKKREGYTDFLAAGGPQNYAWNVVRSEADHLLFQHAASSGAKTFDGVQVKSINFIGEPCEGFGELPYDYPGRPYSATYLMKDDKTSREIKFDYIIDASGRVGLLSTKYLKNRKYNQGLKNVATWGYWKGAAAYGVGTPRQNSPFFEALQDESGWAWLIPLHNGTTSVGIVMNQKMSANRKSQAGSPDSKTFYLGNLKQLAPELSKLLENAELLTDIKSASDYSYSATAYAIPYARIAGDAGCFIDPYFSSGVHLAFVGGLSAATTISAAIKGQVSEVEAADWHSKKVADSYIRFLLVVLSAYRQIRSQEEAVLSDFDEDNYDRAFAFFRPIIQGIADVDTKLSKEELKKTLEFCSNAFEPVKPEDRSSMLEQLGKCPNTAYQVDLSPDQRTVVDHIRARQMMRTEDTMNISSFGTDSINGFVPKLKTGDLGLVAKA

SEQ ID NO: 49 - protein, 425 aa, Artificial Sequence
  SEQ ID NO. 49   HAL8 (prtn_XlOG82Er)
MIDPNSDYDVVVIGGGPGGSATAGLLAQHGHRILLLEREKFPRYHIGESLITGALPTVDALGLRERLDRLGFTKKNGGTLLWGKNTPRSADGQAADPDEGAPGKGTWGFRFREASEYEHSYQVRRADFDALLLGRARELGVHVLEEATVQEPVFDGDRVVGLTYSLKGDARKRTVRSRMVVDASGQTHLLARHFDLVEWQEDLRNIAVWSYLQGCTLYKGDHAGDILTENRPDGWFWFIPLSDGTVSVGYVTPIEKYKESGKSLEDLYVSELAETNEVSRLTSGAFRASGFRTIRDWSYTSSRFHGPGWALVGDAAAFVDPLLSTGVTLAMRGAHGLADAVDAVLRDPQSESHVLDSYEKSYRQFLDSLFDFVRFFYNRSFRKEDYWDKAQATIDPEKVRTRREDFATILSGLTGMRDIFENPSP

SEQ ID NO: 50 - protein, 413 aa, Artificial Sequence
  SEQ ID NO. 50   HAL9 (prtn_HRqXYxIT)
MRQPGAEYDVIVVGGGPAGACTAGLLALEGHRILLVEREKFPRYHVGESLITGIVPTLTALGLLDRMAELRFQVKYGGSLLWGENQTEPWSFRFREVRGGRYEYAWQVRRAEFDAMLLDRARELGVHVIEGATVRDALTDGDRVTGVRYQFKGESDSVSARATIVVDAAGQHRWLGRRFGLVDWYDDLRNVAVWSYWQGALRYPGEHEGDLLTESCRQGWLWYAPLSPELTSIGYVTTSDRLVASGLSPEQLLEKQIAESSEVSWLTSGAKRVDIYRAARDWSYTCQQFSGPGWVLVGDAAAFIDPLLSSGVTLAMRGASSVAKAVHETLTAPDKERHVMKDYEDRYRDFLGQLLELVRFFYDGAHGKEELHLRAQAIVDPDRSLPPKLSFVSLLSGLVRGDESLAPGAIDEH

SEQ ID NO: 51 - protein, 520 aa, Artificial Sequence
  SEQ ID NO. 51   HAL10 (prtn_6X5gftHA)
MEIPEKCTVLVIGGGPGGSFAASALAREGIDTVLLEGDKFPRYHIGESMLASMRHLLRIVDLDSVFDNYGFTKKVGAAFKLNPKKRPGYTDFLAAGGPENYAWNVIRSEADNLMFKHAADSGAKTFDGVQIKAIKFDDIPYKGSVELPHEYPGRPVSATWTRKEDGTSGEIKFDFVVDASGRVGLLSTKYLKNRRYNQGLKNVANWGYWKGCGTYAAGTPRANSPFFEALQDESGWAWAIPLHNGTTSVGIVMNQQMSTDRKVQSGSPDSKTFYLDCLKQLAPDITELLGEGELTSNIKSASDYSYSASGYAIPGARIVGDAGCFIDPYFSSGVHLALVGGLSAATTISASIKGQCTEEAAADWHSKKIADSYTRFLLVVLSAYKQIRSQNAPVLSDFDEDNFDRAFAFFRPVIQGTADVSNKLTQEELNETLEFCSNAFTPVTPEDRNSALEKMGKDITSKEFQVDLSPQEQKVVGHIRARQMMRTEDTINIASFGTDSINGMVPSLQRGSLGLVKSTA

SEQ ID NO: 52 - protein, 528 aa, Artificial Sequence
  SEQ ID NO. 52  HAL11 (prtn_ntNGNnpP)
MSPAIPERCTVLVIGGGPGGSYAASALAREGIDVVVLEGDKFPRYHIGESMLASMRHMLRFIDLEAKFDSYGFIKKPGASFKLNKDKRPGYTDFLAAGGPNNYAWNVVRSEADQLMFHHAGECGAKIFDAVKIKSIRFEDATTVPEGEPNLNPGRPVAAVYEVAETKETGEIAFDYVVDASGRVGLLSTKYMKNRRYNQGLKNVANWGYWEGCNKFSPGTPRENSPFFEALTDESGWAWFIPLHNGKASVGVVMNQKLAAHKKQQGGFNSTEFYHESLKLAPELLSVLVGDGKFVSDVKSASDYSYSASSYAFPNARIVGDAGCFIDPFFSSGVHIALTGALSAATTIAASIRGDSEEATAAEWHSKKVAAAYTRFLLVVLSAYKQMRFQGDPVLSDFDEDNFDRAFSFFRPIIQGTADAANGNLSQDELNKTLEFCAHAFEPVNPEANQEKVMKAVSAAPAGSDYNPDLSVEEQNAVNHIRARKMMRTEDTFNINTFGTDSIVGYVPNLVRGSLGLKKAETNGVVAA

SEQ ID NO: 53 - protein, 534 aa, Artificial Sequence
  SEQ ID NO. 53  HAL12 (prtn_ZiL2dYNe)
MAAQIPKKTTVLVIGGGPGGSYAAAALAREGIDTVVLEGDKFPRYHIGESMLASIRHLLRFVELDAKFDSYGFLKKANTDFLAAGGPDNYAWNVIRSESDKLMFDHAAESGAHVFDGVQVKSVEFEGGVVPGEDGVESLNPGRPISATYLIKETKETGQIAFDYVIDASGRIGILSTKYMKNRRYNQGLKNVANWAYWKGTNPYAPGTTRENSPFFEALQDESGWAWFIPLHNGTTSVGIVENQKLSIEKKQASKTDNSQDFYLENLKLAPNLLALIGDATQVDKVKAASDYSYCASSYAFPYARIVGDAGCFIDPYFSSGVHLALVGGLSAAATICASIRGDVEESSAADWHSKKIADAYTRFLLVVLSAYRQIRSQEEPVLTDINEDHFDRAFAMFRPIIQGTADVGNKKLSQEELKKTLEFCATAFEPVKTEEDRTAAIEEITNNPNGTGYHADLSASQRNAVNHIRARKMMRTEDTVNIDSFGSDAIAGYVPHLKRGSLGLKQVAKGGLDAVPGQMTPVHSHTTAVEVRA

SEQ ID NO: 54 - protein, 528 aa, Artificial Sequence; identical to SEQ ID NO: 52
  SEQ ID NO. 54   HAL13 (prtn_WcV4k6Cx)

SEQ ID NO: 55 - protein, 409 aa, Artificial Sequence
  SEQ ID NO. 55   HAL14 (prtn_IYWELnAD)
MNNGHEEYDVIVVGGGPAGSSAAGLLALNGHSVLLLERDKFPRYHIGESLITGVLPTLARLGLTERMEQLGFQRKYGGSLLWGKGHSKPWTFKFRETGNGKYEYAYQVRRADFDALLLDRARELGVTVLEGASVKEPLFDGDRMVGVRYSVRNEAETVDVSGKITIDATGQQRWLGRHFDMVEWHEDLRNIAAWSYYQGGLRYEGETEGNILTENRPHGWLWYIPLSKDITSIGYVTPSSRLAESGLTPEAMLDKQIAESSEVGRLTANARRVDIYRTARDWSYVCERFSGPGYALVGDAAAFIDPLLSTGVTLAMRGGSTVSRAVHEALVDPAKERDTLQTYEDSYRDFLNSLLEFVRFFYDRTKGQKDYHVGAQAIIDPDKHFPANVDFVTLVSGLARGDDAVDVPS

SEQ ID NO: 56 - protein, 425 aa, Artificial Sequence
  SEQ ID NO. 56 HAL15 (prtn_hZs5MO9S)
MTNTPSAYDVVVIGGGPAGSTTAALLAREGHRVLLLEKEKFPRYHIGESLIPGCLGVVEELGLWDRLEQLGFTKKYGGTLLWGATDGTWDFRFSDGSGYDYSFQARRADFDALLLTRARELGADVVEEATVKDILFDGDRATGLLYTVKGRDEPVRITSRMIVDASGQGRVMGRRLDLVEWHEDLRNVAVWSYFQGCGRISEEKAGDIVVENMWQDENPPGWFWFIPLSDGTVSIGYVTRTALLKESGLSTDQLFEKELAASVEVRKLTADATRVSAYRTLRDWSYECNKFHGPGWAIVGDAAAFVDPLFSTGVTLAMRGALSLSTAVDTALREPAKEAELLKEYEDNYRTFLGHVLDFVRFFYNVKMRKEQYWEGAQEIVDPERKMPPKIDFAALLSGITGQAPSEEILREAKLRSEQIQANQP

SEQ ID NO: 57 - protein, 417 aa, Artificial Sequence
  SEQ ID NO. 57  HAL16 (prtn_aFf3DVs5)
MPNEEPLSEEWDVIVVGGGPAGASTAGLLAQRGHRVLVLEREKFPRYHIGESLITGCVGVLEELGVRERVEALGFVKKFGGSLVWGRDGRWQFKFTEGEPEFPNAYNVRRADFDALILTRARELGAEVREEVTVRKPLVEDERVVGVAYAERGGAECRVRARFVIDCSGQARVLGREMTQVEFHEDLRNVAVWSYFQNGLPLTGDEAGNIFIEHVPGGWFWYIPMYDSTHSVGYVTAAEDASATGLGLPELLETKLKESTHLRQMLENGTRVSAYRSARDWSYACDRMAGPGWMLAGDAAAFVDPLFSTGVTLAMLAAGAAAKAVDCVLCDPAREAAVGERYERSYREFLESILSFVRFFYNAGLDREEYYKYAQALVDPGKLFFPRKDFVTLISGLNSLRPIFDLDTDNLSSSVIA

SEQ ID NO: 58 - protein, 517 aa, Artificial Sequence
  SEQ ID NO. 58  HAL17 (prtn_n6tOqaV6)
MPHEIPERCTVLVVGGGPGGSYCAAALAREGIDTVLLEADRFPRYHIGESMLASMRHLLRFIDLDQTFDQYGFTKKVGAAFKLNKDKQPGYTDFLAAGGPENYAWNVVRSEADNLMFQHAAKCGAKTFDGVQVKSIEFASTDSNPGRPIAASYVHKDDESKETRVISFDYIVDASGRVGFLSTKYLKNRRYNQSLKNVAHWAYWTGAEKYAKGTARENSPFFEALQDESGWAWLIPLHNGTVSVGIVRNQQIASAKKTESGLDLEDFYLDTLLHQAPDIRGLLGDGKLTTAVKSASDYSYSASSYAVPHARIIGDAGCFIDPYFSSGVHLALVGGLSAAITISAALGGDCEETTAAAWHSAKIADSYTRFLLVVLSAYRQIRSQNEPVLSDFGEDNFDRAFAFFRPVIQGTADVHREKLTQDELNRTMEFCANAFEPARPEVRSTVLQKLRAGSEPGDTTFHPDMSPEEERVVKHICARQMMRTEDTMNISSFGTDVINGFLPNLCHGSLGLVRSEV

SEQ ID NO: 59 - protein, 413 aa, Artificial Sequence
  SEQ ID NO. 59  HAL18 (prtn_azOINbIj)
METRSTETADVVIIGGGPAGSTAAGLLAQQGIDVILLEREKFPRYHIGESLITGCVGVIDELGIRDRLENFGFVKKFGGSLVWGKGGRWSFNFAEGDPEFTNAYNVRRADFDAMLLGRARELGTRVLEEAIVKEVLLEDGRVTGVRHAVRGGAETEIRAKLVIDASGQARVLGRHLTEVHWHEDLRNVAVWTYFQGGVSLTGEETGNIFIEHVPGGWFWYIPLHDGTHSVGFVTPANEAGETRLSMPELFTTKIAESVHLKKYLENATRVSAYRNARDWSYACDRFAGPGWMLMGDAAAFVDPLFSTGVTLAMLAARSVAHAVPVMLGNPERAESIARGYEKSYREFLENILSFVRFFYDAGLNREAYYQYAQQLVDPSRLFFPRKDFVTLVSGLNSLNPIFELDVTGTGTRS

SEQ ID NO: 60 - protein, 519 aa, Artificial Sequence
  SEQ ID NO. 60   HAL19 (prtn_9YKhdzV8)
MAIPEKCTVLVVGGGPAGSYAAAALAREGIDTVILEADKFPRYHIGESMLASMRHFLRFIDVDSVFDSYGFTKKVGAAFKLNPKKREGYTDFLAAGGPENYAWNVVRSEADQLLFQHAAKSGAKAFDGVQVKSINFIDVPYKGPGQLPHDYPGRPVSATYVQKDDNTPREIKFDYIIDASGRVGILSTKHLKNRKYNQGLKNVATWGYWKGAGAYGKGTPRENSPFFEALQDESGWAWLIPLHNGTASVGIVMNQKMSVERKTQAGSPDSKTFYLDCLKELAPDLTKLMENGELITDIKSAADYSYSASGYAIPYARIAGDAGCFIDPYFSSGVHLAFVGGLSAAATIAAAIRGDCSEEAAADWHSKKIADAYIRFLLVVLSAYRQIRSQEEPVLSDINEDNFDRAFAFFRPVIQGIADVDTKLSQDELRKTLEFCSNAFEPVKPEDRTAMLEKLGKDPATAYQVDLSDQQRTVVDHIRARQMMRTEDTININSFGTDSINGFVPNLKRGELGLVPAKV

SEQ ID NO: 61 - protein, 517 aa, Artificial Sequence
  SEQ ID NO. 61   HAL20 (prtn_tDIVSZyQ)
MAIPEKCTVLVVGGGPAGSYAASALAREGIDTVILEADKFPRYHIGESMLASMRHFLRFIDVDSEFDNYGFTKKVGAAFKLNPRKREGYTDFLAAGGPENYAWNVVRSEADHLLFKHAAKSGAKAFDGVQIKKINFVDAPYKGSGELPHEYPGRPVSASYLRKDDNTTHEIKFDYIVDASGRVGLLSTKHLKNRRYNQGLKNVANWGYWKGAGAYGKGTPRENSPFFEALQDESGWAWLIPLHNGTVSVGVVMNQKMSAERKSQMGSPDSKTFYLNCLKELAPDLSNLMENGELITDIKSASDYSYSASGYAIPYARIAGDAGCFIDPYFSSGVHLAFVGGLSAATTIAASIRGDCSEEDAADWHSKKVADSYVRFLLVVLSAYRQIRAQNEPVLSDFDEDNFDRAFAFFRPVIQGTADVDSKLSQAELSKTLEFCSNAFEPVKPEDRSSMLEKLGQNPDTAYQVDLSPQQRTVVDHIRARQMMRTEDTMNINSFGTDAINGFIPNLKKGGLGLVRA

SEQ ID NO: 62 - protein, 232 aa, Artificial Sequence
  SEQ ID NO. 62   RFP (prtn_0ImKkjdd)
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 63 - DNA, 6484 bp, Artificial Sequence
  PLAS-400
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatggtgtctaaaggtgaagccgtcatcaaagaattcatgagattcaaggttcacatggaaggttctatgaatggtcacgaattcgaaattgaaggtgaaggtgagggtagaccatatgaaggtactcaaactgctaagttgaaggttacaaaaggtggtccattgccattctcttgggatattttgtctccacaattcatgtacggttctagagcttttacaaaacacccagctgatatcccagattactacaagcaatcatttccagagggtttcaagtgggaaagagttatgaattttgaagatggtggtgccgttactgttactcaagatacttctttggaagatggcaccttgatctacaaggttaagttgagaggtactaatttcccaccagatggtccagttatgcaaaaaaagactatgggttgggaagctagtactgaaagattatatccagaggatggtgttttgaagggtgatattaagatggccttgagattgaaagacggtggtagatatttggctgatttcaagactacttacaaggccaaaaagccagttcaaatgccaggtgcttataacgttgatagaaagttggatatcacctctcacaacgaagattacaccgttgttgaacagtacgaaagatctgaaggtagacattctactggtggtatggatgagttgtacaagtaactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 64 - DNA, 6319 bp, Artificial Sequence
  PLAS-516
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatgacttctacttggtacactttgttggttttggctgttgctgctgaaagattggctgaattggttgttgctagaagaaacactagatggtctttggctagaggtggtgttgaatctggtagaggtcactacccagctatggttgctttgcacactggtttgttgttgggttgtttggctgaagttagattcgctgaaagaccattcccagctgttttgggttggactatggctggtgttgttgttgcttctcaagctttgagatggtggtgtattagaactttgggtccaagatggaacactagagttattgttgttccaggtttgccaagagttactggtggtccatacagatggttgtctcacccaaactacgttgctgttgctgctgaaggtattgctttgccattggttcacggtgcttgggttactgctttggttttcactgctttgaacgctgctttgatggttgttagaattagatgtgaagacggtgctttggctagattgccagctgctgacgctccagcttgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 65 - DNA, 6955 bp, Artificial Sequence
  PLAS-517
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatggaagttatgagagttttgcacatgaacaagggtaacggtgaaacttcttacgctaagaactctactgctcaatctaacattatttctttgggtagaagagttatggacgaagctttgaagaagttgatgatgtctaactctgaaatttcttctattggtattgctgacttgggttgttcttctggtccaaactctttgttgtctatttctaacattgttgacactattcacaacttgtgtccagacttggacagaccagttccagaattgagagtttctttgaacgacttgccatctaacgacttcaactacatttgtgcttctttgccagaattctacgacagagttaacaacaacaaggaaggtttgggtttcggtagaggtggtggtgaatcttgtttcgtttctgctgttccaggttctttctacggtagattgttcccaagaagatctttgcacttcgttcactcttcttcttctttgcactggttgtctcaagttccatgtagagaagctgaaaaggaagacagaactattactgctgacttggaaaacatgggtaagatttacatttctaagacttctccaaagtctgctcacaaggcttacgctttgcaattccaaactgacttctgggttttcttgagatctagatctgaagaattggttccaggtggtagaatggttttgtctttcttgggtagaagatctttggacccaactactgaagaatcttgttaccaatgggaattgttggctcaagctttgatgtctatggctaaggaaggtattattgaagaagaaaagattgacgctttcaacgctccatactacgctgcttcttctgaagaattgaagatggttattgaaaaggaaggttctttctctattgacagattggaaatttctccaattgactgggaaggtggttctatttctgaagaatcttacgacttggttattagatctaagccagaagctttggcttctggtagaagagtttctaacactattagagctgttgttgaaccaatgttggaaccaactttcggtgaaaacgttatggacgaattgttcgaaagatacgctaagattgttggtgaatacttctacgtttcttctccaagatacgctattgttattttgtctttggttagagctggttgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 66 - DNA, 6886 bp, Artificial Sequence
  PLAS-518
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatggctttgccaaacggtatttcttctaagcaagaattgttggaagctcaagctcacgtttggaaccacatttactcttacattaactctatgtctttgaagtgtgctattcaattgggtattccagacgctattcacaagcacggtaacccaattactttgtctcaattggctgacgctttgaacattaacaaggctaagtctcacggtttgttcagattgatgagaattttggttcactctggtttcttcgacaaggttaaggttaaggttaaggttgaaggtgaagacgaagaagaagaagaagacgcttactctttgactccagcttctagattgttgttgagatctgaaccattgtctgttgctccattcgctttggctatgtctgacccagtttacactgaaacttggcaccacttgtctgaatggttcagaaacgacgctgttgctgctttcgacactaagtacggtatgactttcccagaatacgctgttgctgacgacagattgaacgttttgttcaacgaagctatggcttgtgacgctggtttcgttaactctattttgactactgaatgtagagaaattttcgacggtttggaatctatggttgacgttggtggtggtactggtgctactgctaagggtattgctgctgctttcccaggtatggaatgtactgttttggacttgccaaacgttgttggtggtttgaagggttctgaaaacttgtctttcgtttctggtgacatgttcgacttcattccacacgctgacgctattttcatgaagttcattttgcacgactggaacgacgaagaatgtgttaagattttgaagaagtgtaaggaagctatttctagatctaacaactcttgtagaaagattattttggttgaaattgttatggaagacgaaaaggaaactcacgaagctactgaaactaagttgttcttcgacatgcaaatgttggctattattactggtaaggaaagatctgaaaaggaatggggtaagttgttcttcgacgctggtttcactaactacaagattactagagttttgggtttgagatctgttattgaagttttcccatgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 67 - DNA, 6727 bp, Artificial Sequence
  PLAS-519
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatgttgttgagatctagattcttgaaggttattcacgttagaaagcaattgtctgcttgttctagattcgctattcaaactcaaactagatgtaagtctactgacgcttctgaagacgaagttaagcacttccaagaattggctccaacttggtgggacactgacggttctcaaagaattttgcacaagatgaacttgactagattggacttcgttcaaagaactgttagaaaccaagttaagattcaaaacccagaaattttcgttccaggtttcaactacaaggaattcttgccagaatacgtttgtgacaacattcaaagagaaatgcaagaatctattgaaactaacttggacaagagaccagaagtttctgttttggacgttggttgtggtggtggtattttgtctgaatctttggctagattgaagtgggttaagaacgttcaaggtattgacttgactagagactgtattatggttgctaaggaacacgctaagaaggacccaatgttggaaggtaagattaactacgaatgtaaggctttggaagacgttactggtcaattcgacattattacttgtatggaaatgttggaacacgttgacatgccatctgaaattttgagacactgttggtctagattgaacccagaaaagggtattttgttcttgtctactattaacagagacttgatttcttggttcactactattttcatgggtgaaaacgttttgaagattgttccaaagggtactcaccacttgtctaagtacattaactctaaggaaattttggcttggttcaacgacaactactctggtcaattcagattgttggacttgaagggtactatgtacttgccataccaaggttgggttgaacacgactgttctgacgttggtaactacttcatggctattcaaagattgaactaatgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 68 - DNA, 6883 bp, Artificial Sequence
  PLAS-520
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatgggttctactggtgaaactcaaattactccaactcacatttctgacgaagaagctaacttgttcgctatgcaattggcttctgcttctgttttgccaatgattttgaagtctgctttggaattggacttgttggaaattattgctaaggctggtccaggtgctcaaatttctccaattgaaattgcttctcaattgccaactactaacccagacgctccagttatgttggacagaatgttgagattgttggcttgttacattattttgacttgttctgttagaactcaacaagacggtaaggttcaaagattgtacggtttggctactgttgctaagtacttggttaagaacgaagacggtgtttctatttctgctttgaacttgatgaaccaagacaaggttttgatggaatcttggtaccacttgaaggacgctgttttggacggtggtattccattcaacaaggcttacggtatgactgctttcgaataccacggtactgacccaagattcaacaaggttttcaacaagggtatgtctgaccactctactattactatgaagaagattttggaaacttacactggtttcgaaggtttgaagtctttggttgacgttggtggtggtactggtgctgttattaacactattgtttctaagtacccaactattaagggtattaacttcgacttgccacacgttattgaagacgctccatcttacccaggtgttgaacacgttggtggtgacatgttcgtttctattccaaaggctgacgctgttttcatgaagtggatttgtcacgactggtctgacgaacactgtttgaagttcttgaagaactgttacgaagctttgccagacaacggtaaggttattgttgctgaatgtattttgccagttgctccagactcttctttggctactaagggtgttgttcacattgacgttattatgttggctcacaacccaggtggtaaggaaagaactcaaaaggaattcgaagacttggctaagggtgctggtttccaaggtttcaaggttcactgtaacgctttcaacacttacattatggaattcttgaagaaggtttgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 69 - DNA, 6808 bp, Artificial Sequence
  PLAS-521
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatgatttcagtaataattcccttatacaatcaagagaggtatattcaagattgcttggcttctctggccgctcagacaatggacgactttgaggtcattatagttgatgacggtagcacggacagcagcgcagaactagcagccgatttcgcagcacaggacagtagattcacattgatgcgtcaagcaaacgcaggcgtcagcacagcccgtaatcgtggtttagatcaagctaccggtcagtgggtttgtttcgtcgaccctgacgacagtgtcgataaggattacctagctactcttttggcagcaagtgcggaacaacaggatatagatatagtaatgtcaacttgcgttgcgctgactgacgacggagaagtccgtcaacatttcttcccagaatcttttgtcgcacatacagcccaagacaaagaacgtttgttccatcaattgatcgatggtgctttcgaacagagcgacggctttgttaccgcgatcggtgtaccctgggggaagctttacagacgtgatttcctagccgaacatgacttgcgtttcgatccggcgctaccgcgtatgcaggacaatctgtttaatatggaggcatttcaacatgcaagagcaatcacttatttagactatgcgggttaccgttacagagtcggtggcttgtctgagagaacttatcgtaacaccatcaagggcctatatcatccggccatagaccgtcgtgctgaattgatgcacgcatacggtcttgaccaagtaccccaactatatcaagcttggcaggtcgagcaggttaacttgtactatcaagaactgaaagctgcggcgatgctgacggatgggtctgcggcgcaggtcgcgcgtgttgtgagggcaagagccgacacattacgtcagaggaccgcccaagtggacgctgctgtactgccacgtcctatacgtctaaagtatcgtatgcttatcgcaccggccatgacgacccttgctgcactagcgttggcccgtcaacgtcactgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 70 - DNA, 7225 bp, Artificial Sequence
  PLAS-522
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatgaaaactgcagagctagtatttataccagcaccagggatgggacacctggtcccaacagttgaggtagccaaacagctagtagacagacatgaacagctatccattacggtgctaattatgaccattccattggaaacgaacattcccagttacacaaagagtttgtcaagcgattactcttcaaggattacattattgcctttatcacaaccagaaacatcagtcacgatgtcaagttttaatgctatcaattttttcgaatatatttctagttataagggaagggttaaagatgcagtaagtgaaacttcctttagttccagtaatagcgtgaaactagctggttttgtcatcgatatgttctgtaccgctatgattgatgtagctaacgagtttggaattccgtcctatgttttctatacaagtagcgcagctatgctaggcctacaattgcactttcaatccttgtctatagagtgctcaccaaaggttcacaactatgttgaaccagagtcggaagtccttattagtacctacatgaatcctgttccagttaaatgcttaccaggtataattttagttaacgatgaatcctcaaccatgtttgtgaatcacgcacgtagattcagagagaccaagggtattatggtgaatacatttacagaacttgagagccacgcattgaaagcattatctgatgatgagaaaatccctcctatctatccagttggaccgatattgaatcttgaaaacggtaatgaggatcacaaccaagaatatgacgcaatcatgaaatggctagacgagaagccaaattcgtctgtagtttttctgtgttttgggtccaagggttcattcgaagaagaccaggtaaaggagattgcgaacgccttagaatctagcggttaccattttttgtggtcattgagaagaccccctccgaaagataaattacaattccccagtgagtttgaaaacccagaagaagttctacccgaaggcttctttcaaagaactaagggcagaggtaaggtcatcggttgggcacctcaattagctatccttagtcacccctctgtaggcggtttcgtttcacactgtggttggaactccactttagagagcgtaagatccggtgttcccatcgctacttggcctttatacgcagaacaacagagtaacgctttccaattggttaaagatttgggaatggcagtagaaattaaaatggattatagagaggatttcaatacaaggaatccgccactagtaaaagcagaggaaattgaagacggtattaggaaattgatggactctgagaataagattcgtgccaaagttacagagatgaaggacaaatccagagctgcattgctggagggtggctcatcctacgttgccttgggacattttgtcgagaccgtgatgaaaaactagctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 71 - DNA, 7345 bp, Artificial Sequence
  PLAS-523
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatggcgattccggagaaatgcacggtcctagtcgttggcggggggccagccggttcctatgctgcggctgcgctggcaagagagggaattgacaccgttatactggaagctgacaagttccctcgttatcatatcggagagtcaatgttagcgtccatgcgtcatttccttaggtttatagacgtggactctgtattcgattcttacggattcaccaaaaaggtgggagcagcgtttaaacttaaccctaaaaaaagggaaggatatactgacttcctggcagccggaggaccagagaattatgcatggaacgtcgttagaagtgaggcggatcagttacttttccagcacgcagccaaaagtggggcgaaggcgtttgacggggtccaagtgaaatctattaattttatagatgtaccttataaaggacctgggcagctaccacacgactacccgggaaggcctgtaagtgcgacttatgtccaaaaggatgacaacaccccaagggaaataaaattcgattacataatagatgcgagcggaagggttggcatactgagcaccaagcaccttaagaatagaaaatataaccaaggcctgaaaaatgtagcaacttggggttactggaagggtgctggtgcttacggcaaggggacgccaagagaaaacagccccttcttcgaagcacttcaggatgagagcggctgggcgtggttaattccgttgcacaacggaacagcatcagtaggaattgtgatgaatcagaaaatgagtgtggaacgtaagacacaagctgggtcaccagatagtaagactttctacttggactgtcttaaggaactagccccggatcttactaaattaatggagaatggtgagctgattactgacatcaagtccgctgcagactacagctactctgcctcagggtacgctattccctacgcaaggatagcaggtgacgctgggtgctttatagatccctacttctcctccggggtgcatttggcctttgttggcgggctatctgcagcggctactattgctgcggccattaggggcgactgttccgaagaagcagccgcagactggcattcaaaaaaaatcgcggacgcatatatcagatttctgcttgtggttttatcagcctacaggcaaattagatcacaggaggagcccgtattgtccgatatcaacgaggacaactttgatagagctttcgccttctttagacccgttattcaaggaatcgctgacgttgatactaaactttcacaggacgagctacgtaagactcttgaattttgcagcaatgcgtttgagcctgtaaagccggaggacaggaccgcgatgttagagaagttagggaaggacccggctacagcttatcaggtcgacctgagtgaccagcaaaggacagtggtagaccatattcgtgcaaggcaaatgatgagaaccgaagacactataaacattaacagtttcggaactgactccatcaacgggttcgtgcctaatttgaagagaggcgaactaggcctagtaccagctaaggtctgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 72 - DNA, 7339 bp, Artificial Sequence
  PLAS-524
tccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatggctattcccgaaaagtgtacggtactagtggttggtggtggccctgcaggtagttacgctgcttctgccctggcaagagaaggtattgacaccgtgatccttgaggcagataaatttcctagataccatattggagagtcaatgctagccagtatgcgtcattttttgaggttcattgacgtggactcagagtttgataattacgggtttactaagaaagttggtgcagcgttcaaacttaaccctaggaagcgtgaagggtacacagacttcttagcagcgggaggacctgagaactatgcctggaatgtagtcagatctgaggctgaccatctgttatttaagcacgcggccaagtctggagcaaaagcgtttgacggagtccagataaagaaaatcaattttgttgatgctccatacaaaggttccggagagctgccacacgaataccccggccgtccggtgtcagcctcatatctaaggaaggacgacaacaccactcacgaaataaagttcgactacatcgttgacgcgagcggtcgtgtcggtctgctgtcaaccaagcatctgaagaaccgtaggtataatcaagggctaaaaaacgttgccaactggggctattggaagggcgcaggggcatatgggaaaggcactcccagggagaattccccttttttcgaagctcttcaagacgaatcaggctgggcatggctaataccattacacaatggcaccgtgtcagtcggcgttgtgatgaatcagaaaatgagtgctgagaggaagagccaaatggggtcacccgacagcaaaacattttatctaaattgtttaaaggagttagcccccgatctaagtaacctgatggaaaacggggagctgattaccgatatcaagagcgcatcagactactcatacagtgccagtggctatgctatcccttatgcgagaatagccggggatgcaggttgtttcatagacccatatttcagctcaggcgtgcacctggcgttcgttggcgggctgtcagcggctacgacgattgccgcttccatcagaggggactgttctgaagaggacgcggccgattggcactctaagaaggtggcagactcatatgtgcgttttctgttagtcgttctttccgcctataggcaaattcgtgctcagaacgagccagtactttctgacttcgatgaagataactttgatagagctttcgctttcttcagaccggttatccagggaactgctgatgtagacagcaagttatcccaagctgagttaagtaaaacattagaattctgctctaatgcttttgaaccagtaaaacctgaagatagaagttcaatgctagaaaaattaggtcaaaatcccgacactgcctaccaagttgacctaagcccgcaacaaaggactgttgttgaccatattcgtgcccgtcagatgatgaggacggaggacacgatgaacatcaactcatttgggacagatgcaataaacggctttataccgaacctaaaaaaagggggtcttggattggtgagagcatgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaac

SEQ ID NO: 73 - DNA, 3564 bp, Artificial Sequence
  NpgA
tcaatcaaagcaacccacaaatcctaggctgaatcatgatatcgatggaagcaatcaacaattttatcaagaccgcaccaaagcacgactatctgacaggcggagttcatcattctggtaatgtagacgtgttacaattaagcggcaataaagaagatggtagtttagtatggaaccatacttttgttgatgtagacaacaatgtggtagctaagtttgaagacgctctcgaaaaacttgaaagtttgcaccggcgctcatcctcatccacaggcaatgaagaacacgctaacgtttaaccgaggggagtcacttcataatgatgtgagaaataagtgaatattgtaataattgttgggactccattgtcaacaaaagctataatgtaggtatacagtatatactagaagttctcctcgaggatcttggaatccacaaaagggagtcgataaatctatataataaaaattactttatcttctttcgttttatacgttgtcgtttattatcctattacgttatcaatcttcgcatttcagctttcattagatttgatgactgtttctcaaactttatgtcattttcttacaccgcataaacaacaaataatcataaaaattttagaactagacataaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaatctagaaaatttattataaaaggaagagaaataattaaacaatggttcaagatacctcttctgcttctacctctccaattttgactagatggtacattgataccagaccattgactgcttctactgctgctttgccattattggaaactttacaaccagccgatcaaatctccgttcaaaagtactatcacttgaaggacaagcacatgtctttggcttctaacttgttgaagtacttgttcgttcacagaaactgcagaattccatggtcctctatcgttatttctagaactccagatccacatagaaggccatgttatattccaccatctggttctcaagaggattcttttaaagatggttacaccggtatcaacgtcgagtttaatgtttctcatcaagcctccatggttgctattgctggtactgcttttactccaaattctggtggtgattctaagttgaaaccagaagttggtatcgatattacctgcgtcaacgaaagacaaggtagaaatggtgaagaaaggtccttggaatctttgagacagtacatcgatatcttctccgaagttttctctactgctgaaatggccaacattagaagattggatggtgtctcttcttcctcattgtctgctgatagattggttgattatggctacaggttgttctatacttactgggctttgaaagaagcctacattaagatgactggtgaagccttgttggctccatggttgagagaattggaattctctaatgttgttgctccagctgctgttgctgaatctggtgattctgctggtgattttggtgaaccatatactggtgttagaaccaccttgtacaagaacttggttgaagatgttagaattgaagttgctgctttgggtggtgattacttgtttgctactgctgctagaggtggtggtattggtgcttcttctagaccaggtggtggtccagatggttctggtattagatctcaagatccttggaggccattcaagaagttggatattgaaagggatattcaaccatgtgctactggtgtatgtaactgcttgtcttaaagacataaaactgaaacaacaccaattaataatagactttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtccttaaataacatactcatcactaaacattcttaacaatcagaaaatgcaaccgataaaacattataaatcttcgcggttatctggcattgttattaaccaaaaaaatgccggcctattacaagctactgttcaataaatattgttgtaatgaagacggtccaactgtacaaatacagcaaactgtcatatataaggtgtcttatgtgacagcacttgcgttattgtcagccggagtatgtctttgtcgcattctgggctttttactttctgctcagaaggaagtacgaacaagaaaaaaaaatcaccaatgcttcccttttcagtattagtttcatatttgtttacgttcaaactcgtcgtttgcgcgataacctctaaaaaagtcagttacgtaactatatcaatcagagaatgcaaaaagcactatcataaaaatgtctctaggggatgtgagacatgtcaattataagaagtgatggtgtcatagtatatatatcataaatgattatcaaagtttcaatcctttgtattttctagtttagcgccaacttttgacaaaacctaaactttagataatcatcattcttacaatttttatctggatggcaataatctcctatataaagcccagataaactgtaaaaagaatccatcactatttgaaaaaaagtcatctggcacgtttaattatcagagcagaaatgatgaagggtgttagcgccgtccattgatgcgcctggtagtcatgatttacgtataactaacacatcatgaggacggc

SEQ ID NO: 74 - DNA, 11114 bp, Artificial Sequence
  DiPKS[G1516R]-1
tggaccacttcttcttggacttcttaccaccagcaagagcggcagcggctttggcagctttagacaattgttgctttggaggcatgttatattatgttctgagacgtaagaaagggtgaaaattgatgttagtgtcaaaaattatattacaaaatacgcagagatattctagttcctttgatgaatgaatctttcagaaaaaaaagtcaaagcaaaagcaaaatggcctgcagactaaactgtatggtggtcttggaatgataaagatctgtttaatagatttagtagatacaatagcacatctcattacccagttatgattgacgtcattctgagttacaatgatcttaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaataaacaatgaacaagaactccaaaatccagtccccaaactcttctgatgttgctgttattggtgttggttttagattcccaggtaactctaatgacccagaatctttgtggaacaacttgttggatggtttcgatgctattacccaagtcccaaaagaaagatgggctacttcttttagagagatgggtttgatcaagaacaagttcggtggtttcttgaaggattctgaatggaagaatttcgaccctttgttctttggtatcggtccaaaagaagctccattcattgatccacaacaaaggttgttgttgtccatcgtttgggaatctttggaagatgcttacatcagaccagatgaattgagaggttctaacactggtgttttcatcggtgtttctaacaacgattacaccaagttgggtttccaagacaactactctatttctccatacactatgaccggctctaactcttcattgaactccaacagaatttcctactgcttcgattttagaggtccatccattactgttgataccgcttgttcttcttccttggtttctgttaatttgggtgtccaatccatccaaatgggtgaatgtaagattgctatttgcggtggtgttaacgctttgtttgatccatctacatctgttgccttttccaagttgggtgttttgtctgaaaatggcagatgcaactcttttagtgatcaagcctctggttacgttagatctgaaggtgctggtgttgttgttttgaagtctttggaacaagctaagttggatggtgatagaatctacggtgttatcaagggtgtttcctctaatgaagatggtgcttctaatggtgacaagaactctttgactactccatcttgtgaagcccaatccattaacatttctaaggctatggaaaaggcctccttgtctccatctgatatctattacattgaagcccatggtactggtactccagttggtgatccaattgaagttaaggccttgtccaagatcttctccaactctaacaacaaccagttgaacaacttctctaccgatggtaatgataacgatgatgatgatgacgataacacctctccagaaccattattgattggctcattcaagtccaacatcggtcatttggaatctgctgctggtattgcttctttgattaagtgttgcttgatgttgaagaacaggatgttggttccatccattaactgctctaatttgaacccatccattccattcgatcagtacaacatctccgttatcagagaaatcagacaattcccaaccgataagttggttaacatcggtatcaattctttcggtttcggtggttctaactgccatttgattattcaagagtacaacaacaacttcaagaacaactctaccatctgcaataacaacaacaacaacaataacaacatcgactacttgatcccaatctcctctaagactaagaagtccttggataagtacttgattttgatcaagaccaactccaactaccacaaggatatttctttcgatgacttcgtcaagttccaaatcaagtctaagcagtacaacttgtccaacagaatgactaccattgctaacgattggaactccttcattaagggttctaacgaattccacaacttgatcgaatctaaggatggtgaaggtggttcttcatcttctaacagaggtattgattccgccaatcaaatcaacactactactacctctaccatcaacgatatcgaacctttgttggttttcgttttctgtggtcaaggtccacaatggaatggtatgattaagaccttgtacaactccgagaacgttttcaagaacaccgttgatcatgttgacagcatcttgtacaagtacttcggttactccattttgaacgtcttgtctaagatcgatgataacgacgattccatcaaccatccaatagttgctcaaccatctttgttcttgttgcaaattggtttggtcgagttgtttaagtactggggtatctacccatctatctctgttggtcattctttcggtgaagtctcttcttattacttgtccggtatcatctctttggaaaccgcttgtaaaatcgtctacgtcagatcctctaatcagaacaaaactatgggttccggtaagatgttggttgtttctatgggttttaagcaatggaacgatcaattctctgctgaatggtccgatattgaaattgcttgttacaacgctccagattccatagttgttactggtaacgaagaaagattgaaagaattgtccatcaagttgtccgacgaatccaatcaaattttcaacaccttcttgaggtccccatgttcttttcattcttcccatcaagaagtcatcaagggttctatgttcgaagagttgtctaacttgcaatctactggtgaaaccgaaatccctttgttctctactgttactggtagacaagttttgtctggtcatgttactgctcaacacatctacgataatgttagagaaccagtcttgttccaaaagacgattgaatccattacctcctacatcaagtctcactacccatccaatcaaaaggttatctacgttgaaattgctccacacccaaccttgttttcattgatcaaaaagtccatcccatcctccaacaagaattcctcttctgttttgtgtccattgaacagaaaagaaaactccaacaactcctacaagaagttcgtttctcagttgtacttcaacggtgttaacgttgacttcaacttccagttgaactccatttgcgataacgttaacaacgatcaccatttgaacaacgtcaagcaaaactccttcaaagagactaccaattccttgccaagataccaatgggaacaagatgaatattggtccgaaccattgatctccagaaagaatagattggaaggtccaactacttccttgttgggtcatagaattatctacagcttcccagttttccaatccgttttggacttgcaatctgacaactacaaatacttgttggaccacttggttaacggtaagccagtttttccaggtgctggttatttggatatcatcatcgaattcttcgactaccaaaagcagcagttgaattcctctgattcctctaactcctacatcatcaacgttgacaagatccaattcttgaacccaattcacttgaccgaaaacaagttgcaaaccttgcaatcttctttcgaacctatcgttactaagaagtctgccttctctgttaacttcttcatcaaggataccgtcgaggatcaatctaaggttaagtctatgtctgacgaaacttggactaacacttgtaaggctaccatttccttggaacaacaacagccatctccatcttctactttgactttgtctaagaagcaagacttgcagatcttgagaaacagatgcgatattagcaagctagacaagtttgagttgtacgacaagatctctaagaatttgggcttgcagtacaactccttgtttcaagttgttgataccatcgaaactggtaaggattgctcttttgctactttgtctttgccagaagatactttgttcaccaccattttgaacccatgcttgttggataactgtttccatggtttgttgaccttgatcaacgaaaagggttctttcgttgtcgagtccatttcttctgtttctatctacttggagaacatcggttccttcaatcaaacttctgttggtaacgtccagttctacttgtacaccactatttctaaagccacctcctttagttctgaaggtacttgtaagttgttcaccaaggatggttccttgattttgtctatcggtaagttcatcatcaagtccaccaatccaaagtctactaagaccaacgaaactatcgaatctccattggacgaaaccttctctattgaatggcaatctaaggattctccaattccaaccccacaacaaatccaacaacaatctccattgaactctaacccatccttcattagatctaccatcttgaaggacatccagttcgaacaatactgctcctccattatccacaaagaattgatcaaccacgaaaagtacaagaaccagcaatccttcgatatcaactccttggaaaaccacttgaacgatgaccaattgatggaatccttgtccatctccaaagaatacttgagattcttcaccaggatcatctccatcattaagcaatacccaaagatcttgaacgaaaaagagctaaaagaattgaaagaaatcatcgaattgaagtacccatccgaagttcagttgttggaattcgaagttatcgagaaggtgtccatgattatcccaaagttgttgttcgaaaacgacaagcaatcttccatgaccttgttccaagataacttgttgaccaggttctactccaattctaactctaccagattctacttggaaagggtttccgaaatggtcttggaatctattagaccaatcgtcagagaaaagagggtgttcagaattttagagatcggtgctcgtacaggctctttgtctaatgttgttttgactaagttgaacacctacttgtccaccttgaattctaatggtggttctggttacaacatcatcattgagtacaccttcaccgatatttccgccaacttcattattggtgaaatccaagaaaccatgtgcaacttgtacccaaacgttactttcaagttctccgtcttggacttggagaaagagattattaactcctccgatttcttgatgggtgattacgatatagttttgatggcctacgttatccatgccgtttctaacattaagttctccatcgaacagttgtacaagttgttgtctccaagaggttggttgttgtgtattgaacctaagtccaacgttgtgttctccgatttggttttcggttgttttaatcagtggtggaactactacgatgatattagaactacccactgctccttgtctgaatctcaatggaatcagttgttgttgaaccagtccttgaacaacgaatcctcttcttcttctaactgttacggtggtttctccaacgtttcttttattggtggtgaaaaggatgtcgactcccattctttcatattgcactgccaaaaagaatccatctcccaaatgaagttagccaccactattaacaacggtttgtcatctggttccatcgttatcgttttgaactctcaacaattgaccaacatgaagtcctacccaaaggttattgagtatattcaagaggctacctctttgtgcaagaccattgaaattatcgattccaaggacgtcttgaactctaccaattcagttttggaaaagatccaaaagtccttgttggtgttctgtttgttgggttatgacttgttggagaacaactaccaagaacagtctttcgaatacgttaagttgttgaacttgatctctactaccgcctcttcatctaatgataagaaaccaccaaaggtcttgttgatcaccaagcaatctgaaagaatctccaggtctttctactccagatccttgattggtatttccagaacctctatgaacgagtacccaaatttgtccattacctctatcgatttggataccaacgactactcattgcagtctttgttgaagccaatcttcagcaactctaagttttccgacaacgagttcatcttcaaaaagggcttgatgttcgtgtccaggatctttaagaacaagcagttgctagaatcctccaacgcttttgaaactgactcttctaacttgtactgtaaggcctcttctgacttgtcttacaagtacgctattaagcagtctatgttgaccgaaaatcagatcgaaatcaaggttgaatgcgtcggtattaacttcaaggacaacctattctacaagggcttgttgccacaagaaattttcagaatgggtgacatctacaatccaccatatggtttggaatgctctggtgttattaccagaattggttctaacgtcaccgaatactcagttggtcaaaatgtttttggtttcgccagacattctttgggttctcatgttgttaccaacaaggatttggttatcttgaagccagataccatctcattttctgaagctgcttctatcccagttgtttactgtactgcttggtactccttgttcaacattggtcagttgtctaacgaagaatccatcctaattcattctgctactggtggtgtaggtttggcttctttgaatttgttgaaaatgaagaatcagcaacagcaaccattgaccaatgtttatgctactgttggctctaacgagaagaagaagttcttgatcgataacttcaacaacttgttcaaagaggacggcgaaaacattttctctaccagagacaaagaatactccaaccagttggaatccaagatcgatgttattttgaacaccttgtccggtgaattcgtcgaatctaatttcaagtccttgagatccttcggtagattgattgatttgtctgctactcacgtttacgccaatcaacaaattggtctaggtaacttcaagttcgaccacttgtattctgctgttgacttggaaagattgatcgacgaaaaacctaagttgttgcagtccatcttgcaaagaattaccaactctatcgtcaacggttccttggaaaaaattccaattaccatcttcccatccaccgaaactaaggatgctatcgaattattgtccaagagatcccatatcggtaaagttgttgtagattgcaccgatatctctaagtgtaatcctgttggtgatgtgatcaccaacttctctatgagattgccaaagccaaactaccagttgaatttgaactccaccttgttgattactggtcagtctggtttgtctatccctttgttgaattggttgttgtctaagtctggtggtaacgttaagaacgttgtcatcatttctaagtccaccatgaagtggaagttgcagactatgatttcccatttcgtttccggtttcggtatccattttaactacgttcaagtcgacatctccaactacgatgctttgtctgaagctattaagcaattgccatctgatttgccaccaatcacctctgtttttcatttggctgctatctacaacgatgttccaatggatcaagttaccatgtctaccgttgaatctgttcataaccctaaagttttgggtgccgttaacttgcatagaatctctgtttcttttggttggaagttgaaccacttcgtcttgttctcttctattactgctattaccggttacccagaccaatctatctacaattctgccaactctattttggacgctttgtccaactttagaaggtttatgggtttgccatccttctccattaacttgggtccaatgaaggatgaaggtaaggtttctaccaacaagagcatcaagaagctattcaagtctagaggtttgccaagcctatccttgaacaagttatttggtttgttggaggtcgtcatcaacaacccatctaatcatgttatcccatcccaattgatttgctccccaatcgatttcaagacctacatcgaatctttctcaactatgaggccaaagttgttacacttgcaacctaccatttccaagcagcaatcttctatcattaacgattctaccaaggcttcctccaacatttcattgcaagataagatcacctccaaggtgtctgatttgttgtccattccaatctccaagatcaacttcgatcatccattgaaacactacggcttggattctttgttgaccgttcaattcaaatcctggatcgacaaagaattcgaaaagaacttgttcacccatatccaattggccaccatctctattaactcattcttggaaaaggtgaacggcttgtctacaaacaataacaacaacaacaattccaacgtcaagtcctctccatccattgtcaaagaagaaatcgttaccttggacaaggatcaacaaccattgctattgaaagaacaccagcacattatcatctccccagatattagaatcaacaagccaaagagggaatccttgattagaaccccaatcttgaacaaattcaaccagatcaccgaatccattatcactccatctacaccatctttgtcccaatccgatgttttgaaaactccaccaatcaagtctttgaacaacactaagaactccagcttgattaacaccccaccaattcaatctgtccaacaacatcaaaagcaacaacaaaaggtccaagtcatccaacaacagcaacaaccattatccagattgtcctacaagagcaacaacaactctttcgttttgggtatcggtatttctgttccaggtgaacctatttcccaacaatccttgaaagactccatctccaatgacttttctgataaggctgaaactaacgagaaggtcaagagaatctttgagcaatctcaaatcaagaccagacacttggttagagattacactaagccagagaactccatcaagttcagacatttggaaaccattaccgatgtgaacaaccagttcaagaaagttgttccagatttggctcaacaagcctgtttgagagctttgaaagattggggtggtgataagggtgatattacccatatagtttctgttacctccaccggtattatcatcccagatgttaatttcaagttgatcgacttgttgggcttgaacaaggatgttgaaagagtgtctttgaacctaatgggttgtttggctggtttgagttctttgagaactgctgcttctttggctaaggcttctccaagaaatagaattttggttgtctgtaccgaagtctgctccttgcatttttctaatactgatggtggtgatcaaatggtcgcctcttctatttttgctgatggttctgctgcttacattattggttgtaacccaagaattgaagaaaccccattatacgaagtcatgtgctccattaacagatctttcccaaataccgaaaacgccatggtttgggatttggaaaaagaaggttggaacttgggtttggatgcttctattccaattgtcattggttctggtattgaagccttcgttgatactttgttggataaggctaagttgcaaacttccactgctatttctgctaaggattgcgaattcttgattcatactggtggcaagtccatcttgatgaacatcgaaaattccttgggtatcgacccaaagcaaactaagaatacttgggatgtttaccatgcctacggcaatatgtcatctgcctctgttattttcgttatggatcatgccagaaagtccaagtctttgccaacttactcaatttctttggcttttggtccaggtttggcttttgaaggttgtttcttgaagaacgtcgtctgaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctatggacatattccgttcgtacttgagttattggatctatgaaatcgctcgctatacaccagtcatgattttgtcgaagttattatccaggaggcacggatgcaaaagatagaagacaaattaatttccttaaaattcaaaatgctcattattgtcacgctgtatgagcatttggtgaagatttcactgggaaatgttgcaataatttgataatcgttcgtattggatgaaactgtaacatcatctgtttattaagtatccgtgttattagtatatcatcacatacggtgtaagaagataacataaagattgagaaacagtcatcaaatataatggaagctgaaatgcgaggattgatg

SEQ ID NO: 75 - DNA, 10890 bp, Artificial Sequence
  DiPKS[G1516R]-2
acgatgattcagttcgccttctatcctttgtttacgtatttgtttatatatataactttatttttttttattaattgggctgcaagacaattttgttgtcagtgatgcctcaatccttcttttgcttccatatttaccatgtggaccctttcaaaacagagttgtatctctgcaggatgccctttttgacgtattgaatggcataattgcactgtcaaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaataaacaatgaacaagaactccaaaatccagtccccaaactcttctgatgttgctgttattggtgttggttttagattcccaggtaactctaatgacccagaatctttgtggaacaacttgttggatggtttcgatgctattacccaagtcccaaaagaaagatgggctacttcttttagagagatgggtttgatcaagaacaagttcggtggtttcttgaaggattctgaatggaagaatttcgaccctttgttctttggtatcggtccaaaagaagctccattcattgatccacaacaaaggttgttgttgtccatcgtttgggaatctttggaagatgcttacatcagaccagatgaattgagaggttctaacactggtgttttcatcggtgtttctaacaacgattacaccaagttgggtttccaagacaactactctatttctccatacactatgaccggctctaactcttcattgaactccaacagaatttcctactgcttcgattttagaggtccatccattactgttgataccgcttgttcttcttccttggtttctgttaatttgggtgtccaatccatccaaatgggtgaatgtaagattgctatttgcggtggtgttaacgctttgtttgatccatctacatctgttgccttttccaagttgggtgttttgtctgaaaatggcagatgcaactcttttagtgatcaagcctctggttacgttagatctgaaggtgctggtgttgttgttttgaagtctttggaacaagctaagttggatggtgatagaatctacggtgttatcaagggtgtttcctctaatgaagatggtgcttctaatggtgacaagaactctttgactactccatcttgtgaagcccaatccattaacatttctaaggctatggaaaaggcctccttgtctccatctgatatctattacattgaagcccatggtactggtactccagttggtgatccaattgaagttaaggccttgtccaagatcttctccaactctaacaacaaccagttgaacaacttctctaccgatggtaatgataacgatgatgatgatgacgataacacctctccagaaccattattgattggctcattcaagtccaacatcggtcatttggaatctgctgctggtattgcttctttgattaagtgttgcttgatgttgaagaacaggatgttggttccatccattaactgctctaatttgaacccatccattccattcgatcagtacaacatctccgttatcagagaaatcagacaattcccaaccgataagttggttaacatcggtatcaattctttcggtttcggtggttctaactgccatttgattattcaagagtacaacaacaacttcaagaacaactctaccatctgcaataacaacaacaacaacaataacaacatcgactacttgatcccaatctcctctaagactaagaagtccttggataagtacttgattttgatcaagaccaactccaactaccacaaggatatttctttcgatgacttcgtcaagttccaaatcaagtctaagcagtacaacttgtccaacagaatgactaccattgctaacgattggaactccttcattaagggttctaacgaattccacaacttgatcgaatctaaggatggtgaaggtggttcttcatcttctaacagaggtattgattccgccaatcaaatcaacactactactacctctaccatcaacgatatcgaacctttgttggttttcgttttctgtggtcaaggtccacaatggaatggtatgattaagaccttgtacaactccgagaacgttttcaagaacaccgttgatcatgttgacagcatcttgtacaagtacttcggttactccattttgaacgtcttgtctaagatcgatgataacgacgattccatcaaccatccaatagttgctcaaccatctttgttcttgttgcaaattggtttggtcgagttgtttaagtactggggtatctacccatctatctctgttggtcattctttcggtgaagtctcttcttattacttgtccggtatcatctctttggaaaccgcttgtaaaatcgtctacgtcagatcctctaatcagaacaaaactatgggttccggtaagatgttggttgtttctatgggttttaagcaatggaacgatcaattctctgctgaatggtccgatattgaaattgcttgttacaacgctccagattccatagttgttactggtaacgaagaaagattgaaagaattgtccatcaagttgtccgacgaatccaatcaaattttcaacaccttcttgaggtccccatgttcttttcattcttcccatcaagaagtcatcaagggttctatgttcgaagagttgtctaacttgcaatctactggtgaaaccgaaatccctttgttctctactgttactggtagacaagttttgtctggtcatgttactgctcaacacatctacgataatgttagagaaccagtcttgttccaaaagacgattgaatccattacctcctacatcaagtctcactacccatccaatcaaaaggttatctacgttgaaattgctccacacccaaccttgttttcattgatcaaaaagtccatcccatcctccaacaagaattcctcttctgttttgtgtccattgaacagaaaagaaaactccaacaactcctacaagaagttcgtttctcagttgtacttcaacggtgttaacgttgacttcaacttccagttgaactccatttgcgataacgttaacaacgatcaccatttgaacaacgtcaagcaaaactccttcaaagagactaccaattccttgccaagataccaatgggaacaagatgaatattggtccgaaccattgatctccagaaagaatagattggaaggtccaactacttccttgttgggtcatagaattatctacagcttcccagttttccaatccgttttggacttgcaatctgacaactacaaatacttgttggaccacttggttaacggtaagccagtttttccaggtgctggttatttggatatcatcatcgaattcttcgactaccaaaagcagcagttgaattcctctgattcctctaactcctacatcatcaacgttgacaagatccaattcttgaacccaattcacttgaccgaaaacaagttgcaaaccttgcaatcttctttcgaacctatcgttactaagaagtctgccttctctgttaacttcttcatcaaggataccgtcgaggatcaatctaaggttaagtctatgtctgacgaaacttggactaacacttgtaaggctaccatttccttggaacaacaacagccatctccatcttctactttgactttgtctaagaagcaagacttgcagatcttgagaaacagatgcgatattagcaagctagacaagtttgagttgtacgacaagatctctaagaatttgggcttgcagtacaactccttgtttcaagttgttgataccatcgaaactggtaaggattgctcttttgctactttgtctttgccagaagatactttgttcaccaccattttgaacccatgcttgttggataactgtttccatggtttgttgaccttgatcaacgaaaagggttctttcgttgtcgagtccatttcttctgtttctatctacttggagaacatcggttccttcaatcaaacttctgttggtaacgtccagttctacttgtacaccactatttctaaagccacctcctttagttctgaaggtacttgtaagttgttcaccaaggatggttccttgattttgtctatcggtaagttcatcatcaagtccaccaatccaaagtctactaagaccaacgaaactatcgaatctccattggacgaaaccttctctattgaatggcaatctaaggattctccaattccaaccccacaacaaatccaacaacaatctccattgaactctaacccatccttcattagatctaccatcttgaaggacatccagttcgaacaatactgctcctccattatccacaaagaattgatcaaccacgaaaagtacaagaaccagcaatccttcgatatcaactccttggaaaaccacttgaacgatgaccaattgatggaatccttgtccatctccaaagaatacttgagattcttcaccaggatcatctccatcattaagcaatacccaaagatcttgaacgaaaaagagctaaaagaattgaaagaaatcatcgaattgaagtacccatccgaagttcagttgttggaattcgaagttatcgagaaggtgtccatgattatcccaaagttgttgttcgaaaacgacaagcaatcttccatgaccttgttccaagataacttgttgaccaggttctactccaattctaactctaccagattctacttggaaagggtttccgaaatggtcttggaatctattagaccaatcgtcagagaaaagagggtgttcagaattttagagatcggtgctcgtacaggctctttgtctaatgttgttttgactaagttgaacacctacttgtccaccttgaattctaatggtggttctggttacaacatcatcattgagtacaccttcaccgatatttccgccaacttcattattggtgaaatccaagaaaccatgtgcaacttgtacccaaacgttactttcaagttctccgtcttggacttggagaaagagattattaactcctccgatttcttgatgggtgattacgatatagttttgatggcctacgttatccatgccgtttctaacattaagttctccatcgaacagttgtacaagttgttgtctccaagaggttggttgttgtgtattgaacctaagtccaacgttgtgttctccgatttggttttcggttgttttaatcagtggtggaactactacgatgatattagaactacccactgctccttgtctgaatctcaatggaatcagttgttgttgaaccagtccttgaacaacgaatcctcttcttcttctaactgttacggtggtttctccaacgtttcttttattggtggtgaaaaggatgtcgactcccattctttcatattgcactgccaaaaagaatccatctcccaaatgaagttagccaccactattaacaacggtttgtcatctggttccatcgttatcgttttgaactctcaacaattgaccaacatgaagtcctacccaaaggttattgagtatattcaagaggctacctctttgtgcaagaccattgaaattatcgattccaaggacgtcttgaactctaccaattcagttttggaaaagatccaaaagtccttgttggtgttctgtttgttgggttatgacttgttggagaacaactaccaagaacagtctttcgaatacgttaagttgttgaacttgatctctactaccgcctcttcatctaatgataagaaaccaccaaaggtcttgttgatcaccaagcaatctgaaagaatctccaggtctttctactccagatccttgattggtatttccagaacctctatgaacgagtacccaaatttgtccattacctctatcgatttggataccaacgactactcattgcagtctttgttgaagccaatcttcagcaactctaagttttccgacaacgagttcatcttcaaaaagggcttgatgttcgtgtccaggatctttaagaacaagcagttgctagaatcctccaacgcttttgaaactgactcttctaacttgtactgtaaggcctcttctgacttgtcttacaagtacgctattaagcagtctatgttgaccgaaaatcagatcgaaatcaaggttgaatgcgtcggtattaacttcaaggacaacctattctacaagggcttgttgccacaagaaattttcagaatgggtgacatctacaatccaccatatggtttggaatgctctggtgttattaccagaattggttctaacgtcaccgaatactcagttggtcaaaatgtttttggtttcgccagacattctttgggttctcatgttgttaccaacaaggatttggttatcttgaagccagataccatctcattttctgaagctgcttctatcccagttgtttactgtactgcttggtactccttgttcaacattggtcagttgtctaacgaagaatccatcctaattcattctgctactggtggtgtaggtttggcttctttgaatttgttgaaaatgaagaatcagcaacagcaaccattgaccaatgtttatgctactgttggctctaacgagaagaagaagttcttgatcgataacttcaacaacttgttcaaagaggacggcgaaaacattttctctaccagagacaaagaatactccaaccagttggaatccaagatcgatgttattttgaacaccttgtccggtgaattcgtcgaatctaatttcaagtccttgagatccttcggtagattgattgatttgtctgctactcacgtttacgccaatcaacaaattggtctaggtaacttcaagttcgaccacttgtattctgctgttgacttggaaagattgatcgacgaaaaacctaagttgttgcagtccatcttgcaaagaattaccaactctatcgtcaacggttccttggaaaaaattccaattaccatcttcccatccaccgaaactaaggatgctatcgaattattgtccaagagatcccatatcggtaaagttgttgtagattgcaccgatatctctaagtgtaatcctgttggtgatgtgatcaccaacttctctatgagattgccaaagccaaactaccagttgaatttgaactccaccttgttgattactggtcagtctggtttgtctatccctttgttgaattggttgttgtctaagtctggtggtaacgttaagaacgttgtcatcatttctaagtccaccatgaagtggaagttgcagactatgatttcccatttcgtttccggtttcggtatccattttaactacgttcaagtcgacatctccaactacgatgctttgtctgaagctattaagcaattgccatctgatttgccaccaatcacctctgtttttcatttggctgctatctacaacgatgttccaatggatcaagttaccatgtctaccgttgaatctgttcataaccctaaagttttgggtgccgttaacttgcatagaatctctgtttcttttggttggaagttgaaccacttcgtcttgttctcttctattactgctattaccggttacccagaccaatctatctacaattctgccaactctattttggacgctttgtccaactttagaaggtttatgggtttgccatccttctccattaacttgggtccaatgaaggatgaaggtaaggtttctaccaacaagagcatcaagaagctattcaagtctagaggtttgccaagcctatccttgaacaagttatttggtttgttggaggtcgtcatcaacaacccatctaatcatgttatcccatcccaattgatttgctccccaatcgatttcaagacctacatcgaatctttctcaactatgaggccaaagttgttacacttgcaacctaccatttccaagcagcaatcttctatcattaacgattctaccaaggcttcctccaacatttcattgcaagataagatcacctccaaggtgtctgatttgttgtccattccaatctccaagatcaacttcgatcatccattgaaacactacggcttggattctttgttgaccgttcaattcaaatcctggatcgacaaagaattcgaaaagaacttgttcacccatatccaattggccaccatctctattaactcattcttggaaaaggtgaacggcttgtctacaaacaataacaacaacaacaattccaacgtcaagtcctctccatccattgtcaaagaagaaatcgttaccttggacaaggatcaacaaccattgctattgaaagaacaccagcacattatcatctccccagatattagaatcaacaagccaaagagggaatccttgattagaaccccaatcttgaacaaattcaaccagatcaccgaatccattatcactccatctacaccatctttgtcccaatccgatgttttgaaaactccaccaatcaagtctttgaacaacactaagaactccagcttgattaacaccccaccaattcaatctgtccaacaacatcaaaagcaacaacaaaaggtccaagtcatccaacaacagcaacaaccattatccagattgtcctacaagagcaacaacaactctttcgttttgggtatcggtatttctgttccaggtgaacctatttcccaacaatccttgaaagactccatctccaatgacttttctgataaggctgaaactaacgagaaggtcaagagaatctttgagcaatctcaaatcaagaccagacacttggttagagattacactaagccagagaactccatcaagttcagacatttggaaaccattaccgatgtgaacaaccagttcaagaaagttgttccagatttggctcaacaagcctgtttgagagctttgaaagattggggtggtgataagggtgatattacccatatagtttctgttacctccaccggtattatcatcccagatgttaatttcaagttgatcgacttgttgggcttgaacaaggatgttgaaagagtgtctttgaacctaatgggttgtttggctggtttgagttctttgagaactgctgcttctttggctaaggcttctccaagaaatagaattttggttgtctgtaccgaagtctgctccttgcatttttctaatactgatggtggtgatcaaatggtcgcctcttctatttttgctgatggttctgctgcttacattattggttgtaacccaagaattgaagaaaccccattatacgaagtcatgtgctccattaacagatctttcccaaataccgaaaacgccatggtttgggatttggaaaaagaaggttggaacttgggtttggatgcttctattccaattgtcattggttctggtattgaagccttcgttgatactttgttggataaggctaagttgcaaacttccactgctatttctgctaaggattgcgaattcttgattcatactggtggcaagtccatcttgatgaacatcgaaaattccttgggtatcgacccaaagcaaactaagaatacttgggatgtttaccatgcctacggcaatatgtcatctgcctctgttattttcgttatggatcatgccagaaagtccaagtctttgccaacttactcaatttctttggcttttggtccaggtttggcttttgaaggttgtttcttgaagaacgtcgtctgaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtctatgtgttgctcttaaaatatttggatacgacatcctttatcttttttcctttaagagcaggatataagccatcaagtttctgaaaatcaaaatggtagcaacaataatgcagacgacaacaactgtgctgacgacagtcgccgcaatgtctactaccttagcatcaaattacatatcttcgcaagctagtt

SEQ ID NO: 76 - DNA, 11300 bp, Artificial Sequence
  DiPKS[G1516R]-3
cgggttacagtcatcgttgaaaacgatggaaaaacatgtcgagattcctcaatccatacaccattatagtccgttttatcagcttccactaattttttaaatctcagtttcttcttgaaatttagcatcgtgcatgggatagcggctagtaaaaaagaaaattaatatctcattaacaaagttattgtacataatccggtacaatattcttcaatgtactctctaatatcgagcacactggcaatattcatgcacacattcgcctaatgctgacgaatgcttaatcagtgcaattaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaataaacaatgaacaagaactccaaaatccagtccccaaactcttctgatgttgctgttattggtgttggttttagattcccaggtaactctaatgacccagaatctttgtggaacaacttgttggatggtttcgatgctattacccaagtcccaaaagaaagatgggctacttcttttagagagatgggtttgatcaagaacaagttcggtggtttcttgaaggattctgaatggaagaatttcgaccctttgttctttggtatcggtccaaaagaagctccattcattgatccacaacaaaggttgttgttgtccatcgtttgggaatctttggaagatgcttacatcagaccagatgaattgagaggttctaacactggtgttttcatcggtgtttctaacaacgattacaccaagttgggtttccaagacaactactctatttctccatacactatgaccggctctaactcttcattgaactccaacagaatttcctactgcttcgattttagaggtccatccattactgttgataccgcttgttcttcttccttggtttctgttaatttgggtgtccaatccatccaaatgggtgaatgtaagattgctatttgcggtggtgttaacgctttgtttgatccatctacatctgttgccttttccaagttgggtgttttgtctgaaaatggcagatgcaactcttttagtgatcaagcctctggttacgttagatctgaaggtgctggtgttgttgttttgaagtctttggaacaagctaagttggatggtgatagaatctacggtgttatcaagggtgtttcctctaatgaagatggtgcttctaatggtgacaagaactctttgactactccatcttgtgaagcccaatccattaacatttctaaggctatggaaaaggcctccttgtctccatctgatatctattacattgaagcccatggtactggtactccagttggtgatccaattgaagttaaggccttgtccaagatcttctccaactctaacaacaaccagttgaacaacttctctaccgatggtaatgataacgatgatgatgatgacgataacacctctccagaaccattattgattggctcattcaagtccaacatcggtcatttggaatctgctgctggtattgcttctttgattaagtgttgcttgatgttgaagaacaggatgttggttccatccattaactgctctaatttgaacccatccattccattcgatcagtacaacatctccgttatcagagaaatcagacaattcccaaccgataagttggttaacatcggtatcaattctttcggtttcggtggttctaactgccatttgattattcaagagtacaacaacaacttcaagaacaactctaccatctgcaataacaacaacaacaacaataacaacatcgactacttgatcccaatctcctctaagactaagaagtccttggataagtacttgattttgatcaagaccaactccaactaccacaaggatatttctttcgatgacttcgtcaagttccaaatcaagtctaagcagtacaacttgtccaacagaatgactaccattgctaacgattggaactccttcattaagggttctaacgaattccacaacttgatcgaatctaaggatggtgaaggtggttcttcatcttctaacagaggtattgattccgccaatcaaatcaacactactactacctctaccatcaacgatatcgaacctttgttggttttcgttttctgtggtcaaggtccacaatggaatggtatgattaagaccttgtacaactccgagaacgttttcaagaacaccgttgatcatgttgacagcatcttgtacaagtacttcggttactccattttgaacgtcttgtctaagatcgatgataacgacgattccatcaaccatccaatagttgctcaaccatctttgttcttgttgcaaattggtttggtcgagttgtttaagtactggggtatctacccatctatctctgttggtcattctttcggtgaagtctcttcttattacttgtccggtatcatctctttggaaaccgcttgtaaaatcgtctacgtcagatcctctaatcagaacaaaactatgggttccggtaagatgttggttgtttctatgggttttaagcaatggaacgatcaattctctgctgaatggtccgatattgaaattgcttgttacaacgctccagattccatagttgttactggtaacgaagaaagattgaaagaattgtccatcaagttgtccgacgaatccaatcaaattttcaacaccttcttgaggtccccatgttcttttcattcttcccatcaagaagtcatcaagggttctatgttcgaagagttgtctaacttgcaatctactggtgaaaccgaaatccctttgttctctactgttactggtagacaagttttgtctggtcatgttactgctcaacacatctacgataatgttagagaaccagtcttgttccaaaagacgattgaatccattacctcctacatcaagtctcactacccatccaatcaaaaggttatctacgttgaaattgctccacacccaaccttgttttcattgatcaaaaagtccatcccatcctccaacaagaattcctcttctgttttgtgtccattgaacagaaaagaaaactccaacaactcctacaagaagttcgtttctcagttgtacttcaacggtgttaacgttgacttcaacttccagttgaactccatttgcgataacgttaacaacgatcaccatttgaacaacgtcaagcaaaactccttcaaagagactaccaattccttgccaagataccaatgggaacaagatgaatattggtccgaaccattgatctccagaaagaatagattggaaggtccaactacttccttgttgggtcatagaattatctacagcttcccagttttccaatccgttttggacttgcaatctgacaactacaaatacttgttggaccacttggttaacggtaagccagtttttccaggtgctggttatttggatatcatcatcgaattcttcgactaccaaaagcagcagttgaattcctctgattcctctaactcctacatcatcaacgttgacaagatccaattcttgaacccaattcacttgaccgaaaacaagttgcaaaccttgcaatcttctttcgaacctatcgttactaagaagtctgccttctctgttaacttcttcatcaaggataccgtcgaggatcaatctaaggttaagtctatgtctgacgaaacttggactaacacttgtaaggctaccatttccttggaacaacaacagccatctccatcttctactttgactttgtctaagaagcaagacttgcagatcttgagaaacagatgcgatattagcaagctagacaagtttgagttgtacgacaagatctctaagaatttgggcttgcagtacaactccttgtttcaagttgttgataccatcgaaactggtaaggattgctcttttgctactttgtctttgccagaagatactttgttcaccaccattttgaacccatgcttgttggataactgtttccatggtttgttgaccttgatcaacgaaaagggttctttcgttgtcgagtccatttcttctgtttctatctacttggagaacatcggttccttcaatcaaacttctgttggtaacgtccagttctacttgtacaccactatttctaaagccacctcctttagttctgaaggtacttgtaagttgttcaccaaggatggttccttgattttgtctatcggtaagttcatcatcaagtccaccaatccaaagtctactaagaccaacgaaactatcgaatctccattggacgaaaccttctctattgaatggcaatctaaggattctccaattccaaccccacaacaaatccaacaacaatctccattgaactctaacccatccttcattagatctaccatcttgaaggacatccagttcgaacaatactgctcctccattatccacaaagaattgatcaaccacgaaaagtacaagaaccagcaatccttcgatatcaactccttggaaaaccacttgaacgatgaccaattgatggaatccttgtccatctccaaagaatacttgagattcttcaccaggatcatctccatcattaagcaatacccaaagatcttgaacgaaaaagagctaaaagaattgaaagaaatcatcgaattgaagtacccatccgaagttcagttgttggaattcgaagttatcgagaaggtgtccatgattatcccaaagttgttgttcgaaaacgacaagcaatcttccatgaccttgttccaagataacttgttgaccaggttctactccaattctaactctaccagattctacttggaaagggtttccgaaatggtcttggaatctattagaccaatcgtcagagaaaagagggtgttcagaattttagagatcggtgctcgtacaggctctttgtctaatgttgttttgactaagttgaacacctacttgtccaccttgaattctaatggtggttctggttacaacatcatcattgagtacaccttcaccgatatttccgccaacttcattattggtgaaatccaagaaaccatgtgcaacttgtacccaaacgttactttcaagttctccgtcttggacttggagaaagagattattaactcctccgatttcttgatgggtgattacgatatagttttgatggcctacgttatccatgccgtttctaacattaagttctccatcgaacagttgtacaagttgttgtctccaagaggttggttgttgtgtattgaacctaagtccaacgttgtgttctccgatttggttttcggttgttttaatcagtggtggaactactacgatgatattagaactacccactgctccttgtctgaatctcaatggaatcagttgttgttgaaccagtccttgaacaacgaatcctcttcttcttctaactgttacggtggtttctccaacgtttcttttattggtggtgaaaaggatgtcgactcccattctttcatattgcactgccaaaaagaatccatctcccaaatgaagttagccaccactattaacaacggtttgtcatctggttccatcgttatcgttttgaactctcaacaattgaccaacatgaagtcctacccaaaggttattgagtatattcaagaggctacctctttgtgcaagaccattgaaattatcgattccaaggacgtcttgaactctaccaattcagttttggaaaagatccaaaagtccttgttggtgttctgtttgttgggttatgacttgttggagaacaactaccaagaacagtctttcgaatacgttaagttgttgaacttgatctctactaccgcctcttcatctaatgataagaaaccaccaaaggtcttgttgatcaccaagcaatctgaaagaatctccaggtctttctactccagatccttgattggtatttccagaacctctatgaacgagtacccaaatttgtccattacctctatcgatttggataccaacgactactcattgcagtctttgttgaagccaatcttcagcaactctaagttttccgacaacgagttcatcttcaaaaagggcttgatgttcgtgtccaggatctttaagaacaagcagttgctagaatcctccaacgcttttgaaactgactcttctaacttgtactgtaaggcctcttctgacttgtcttacaagtacgctattaagcagtctatgttgaccgaaaatcagatcgaaatcaaggttgaatgcgtcggtattaacttcaaggacaacctattctacaagggcttgttgccacaagaaattttcagaatgggtgacatctacaatccaccatatggtttggaatgctctggtgttattaccagaattggttctaacgtcaccgaatactcagttggtcaaaatgtttttggtttcgccagacattctttgggttctcatgttgttaccaacaaggatttggttatcttgaagccagataccatctcattttctgaagctgcttctatcccagttgtttactgtactgcttggtactccttgttcaacattggtcagttgtctaacgaagaatccatcctaattcattctgctactggtggtgtaggtttggcttctttgaatttgttgaaaatgaagaatcagcaacagcaaccattgaccaatgtttatgctactgttggctctaacgagaagaagaagttcttgatcgataacttcaacaacttgttcaaagaggacggcgaaaacattttctctaccagagacaaagaatactccaaccagttggaatccaagatcgatgttattttgaacaccttgtccggtgaattcgtcgaatctaatttcaagtccttgagatccttcggtagattgattgatttgtctgctactcacgtttacgccaatcaacaaattggtctaggtaacttcaagttcgaccacttgtattctgctgttgacttggaaagattgatcgacgaaaaacctaagttgttgcagtccatcttgcaaagaattaccaactctatcgtcaacggttccttggaaaaaattccaattaccatcttcccatccaccgaaactaaggatgctatcgaattattgtccaagagatcccatatcggtaaagttgttgtagattgcaccgatatctctaagtgtaatcctgttggtgatgtgatcaccaacttctctatgagattgccaaagccaaactaccagttgaatttgaactccaccttgttgattactggtcagtctggtttgtctatccctttgttgaattggttgttgtctaagtctggtggtaacgttaagaacgttgtcatcatttctaagtccaccatgaagtggaagttgcagactatgatttcccatttcgtttccggtttcggtatccattttaactacgttcaagtcgacatctccaactacgatgctttgtctgaagctattaagcaattgccatctgatttgccaccaatcacctctgtttttcatttggctgctatctacaacgatgttccaatggatcaagttaccatgtctaccgttgaatctgttcataaccctaaagttttgggtgccgttaacttgcatagaatctctgtttcttttggttggaagttgaaccacttcgtcttgttctcttctattactgctattaccggttacccagaccaatctatctacaattctgccaactctattttggacgctttgtccaactttagaaggtttatgggtttgccatccttctccattaacttgggtccaatgaaggatgaaggtaaggtttctaccaacaagagcatcaagaagctattcaagtctagaggtttgccaagcctatccttgaacaagttatttggtttgttggaggtcgtcatcaacaacccatctaatcatgttatcccatcccaattgatttgctccccaatcgatttcaagacctacatcgaatctttctcaactatgaggccaaagttgttacacttgcaacctaccatttccaagcagcaatcttctatcattaacgattctaccaaggcttcctccaacatttcattgcaagataagatcacctccaaggtgtctgatttgttgtccattccaatctccaagatcaacttcgatcatccattgaaacactacggcttggattctttgttgaccgttcaattcaaatcctggatcgacaaagaattcgaaaagaacttgttcacccatatccaattggccaccatctctattaactcattcttggaaaaggtgaacggcttgtctacaaacaataacaacaacaacaattccaacgtcaagtcctctccatccattgtcaaagaagaaatcgttaccttggacaaggatcaacaaccattgctattgaaagaacaccagcacattatcatctccccagatattagaatcaacaagccaaagagggaatccttgattagaaccccaatcttgaacaaattcaaccagatcaccgaatccattatcactccatctacaccatctttgtcccaatccgatgttttgaaaactccaccaatcaagtctttgaacaacactaagaactccagcttgattaacaccccaccaattcaatctgtccaacaacatcaaaagcaacaacaaaaggtccaagtcatccaacaacagcaacaaccattatccagattgtcctacaagagcaacaacaactctttcgttttgggtatcggtatttctgttccaggtgaacctatttcccaacaatccttgaaagactccatctccaatgacttttctgataaggctgaaactaacgagaaggtcaagagaatctttgagcaatctcaaatcaagaccagacacttggttagagattacactaagccagagaactccatcaagttcagacatttggaaaccattaccgatgtgaacaaccagttcaagaaagttgttccagatttggctcaacaagcctgtttgagagctttgaaagattggggtggtgataagggtgatattacccatatagtttctgttacctccaccggtattatcatcccagatgttaatttcaagttgatcgacttgttgggcttgaacaaggatgttgaaagagtgtctttgaacctaatgggttgtttggctggtttgagttctttgagaactgctgcttctttggctaaggcttctccaagaaatagaattttggttgtctgtaccgaagtctgctccttgcatttttctaatactgatggtggtgatcaaatggtcgcctcttctatttttgctgatggttctgctgcttacattattggttgtaacccaagaattgaagaaaccccattatacgaagtcatgtgctccattaacagatctttcccaaataccgaaaacgccatggtttgggatttggaaaaagaaggttggaacttgggtttggatgcttctattccaattgtcattggttctggtattgaagccttcgttgatactttgttggataaggctaagttgcaaacttccactgctatttctgctaaggattgcgaattcttgattcatactggtggcaagtccatcttgatgaacatcgaaaattccttgggtatcgacccaaagcaaactaagaatacttgggatgtttaccatgcctacggcaatatgtcatctgcctctgttattttcgttatggatcatgccagaaagtccaagtctttgccaacttactcaatttctttggcttttggtccaggtttggcttttgaaggttgtttcttgaagaacgtcgtctgaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtctgagatcgacttgcatcaacctttgaaaaatataaggatgagaaagtgaaatcggttttttttttccattgtcgtcatcaacatgattttttaaataaataaatacgattttttattttttttcccttctttgtttttgttttgcttattcccatcttcattattaaattcttccgctcttaataaaggagtttttttattatcttcttgtgtaatcatcctttttctttaattttcttccttttctttttctctttactggtttttttacttctttattctcaaccatctaaagaatattattgctttctaccaataaaatctgttaattctatttggattgtcgtctactcaagtctcgcctagtaaataaacgataaacaaatttgaagtaagaataacaatatagggagagaaatttttctatttttaatttcgaaacaggtaccaaaaaatctaagttcactttagcactatttgggaaagcttttatataaaaaatctgaaacaaaatcatatcaaag

SEQ ID NO: 77 - DNA, 11140 bp, Artificial Sequence
  DiPKS[G1516R]-4
cggcacccaccataaagattactttagcgttggggttgcttttcatatgagcagcgatcttcctaaccgacatctctgtagaggcggtagaaacagacatcgaaacacggagagatcgcggtacgtctggttggtagctcagtgacgggcacagatagcgacatttactgatacagagacagttacgaaagatggaaacacatgcagagaatgtggtctattccttattcttaagatcggcgaagctaacagagttaatgctgggccatagctggacggcagaaagcacgcaggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaataaacaatgaacaagaactccaaaatccagtccccaaactcttctgatgttgctgttattggtgttggttttagattcccaggtaactctaatgacccagaatctttgtggaacaacttgttggatggtttcgatgctattacccaagtcccaaaagaaagatgggctacttcttttagagagatgggtttgatcaagaacaagttcggtggtttcttgaaggattctgaatggaagaatttcgaccctttgttctttggtatcggtccaaaagaagctccattcattgatccacaacaaaggttgttgttgtccatcgtttgggaatctttggaagatgcttacatcagaccagatgaattgagaggttctaacactggtgttttcatcggtgtttctaacaacgattacaccaagttgggtttccaagacaactactctatttctccatacactatgaccggctctaactcttcattgaactccaacagaatttcctactgcttcgattttagaggtccatccattactgttgataccgcttgttcttcttccttggtttctgttaatttgggtgtccaatccatccaaatgggtgaatgtaagattgctatttgcggtggtgttaacgctttgtttgatccatctacatctgttgccttttccaagttgggtgttttgtctgaaaatggcagatgcaactcttttagtgatcaagcctctggttacgttagatctgaaggtgctggtgttgttgttttgaagtctttggaacaagctaagttggatggtgatagaatctacggtgttatcaagggtgtttcctctaatgaagatggtgcttctaatggtgacaagaactctttgactactccatcttgtgaagcccaatccattaacatttctaaggctatggaaaaggcctccttgtctccatctgatatctattacattgaagcccatggtactggtactccagttggtgatccaattgaagttaaggccttgtccaagatcttctccaactctaacaacaaccagttgaacaacttctctaccgatggtaatgataacgatgatgatgatgacgataacacctctccagaaccattattgattggctcattcaagtccaacatcggtcatttggaatctgctgctggtattgcttctttgattaagtgttgcttgatgttgaagaacaggatgttggttccatccattaactgctctaatttgaacccatccattccattcgatcagtacaacatctccgttatcagagaaatcagacaattcccaaccgataagttggttaacatcggtatcaattctttcggtttcggtggttctaactgccatttgattattcaagagtacaacaacaacttcaagaacaactctaccatctgcaataacaacaacaacaacaataacaacatcgactacttgatcccaatctcctctaagactaagaagtccttggataagtacttgattttgatcaagaccaactccaactaccacaaggatatttctttcgatgacttcgtcaagttccaaatcaagtctaagcagtacaacttgtccaacagaatgactaccattgctaacgattggaactccttcattaagggttctaacgaattccacaacttgatcgaatctaaggatggtgaaggtggttcttcatcttctaacagaggtattgattccgccaatcaaatcaacactactactacctctaccatcaacgatatcgaacctttgttggttttcgttttctgtggtcaaggtccacaatggaatggtatgattaagaccttgtacaactccgagaacgttttcaagaacaccgttgatcatgttgacagcatcttgtacaagtacttcggttactccattttgaacgtcttgtctaagatcgatgataacgacgattccatcaaccatccaatagttgctcaaccatctttgttcttgttgcaaattggtttggtcgagttgtttaagtactggggtatctacccatctatctctgttggtcattctttcggtgaagtctcttcttattacttgtccggtatcatctctttggaaaccgcttgtaaaatcgtctacgtcagatcctctaatcagaacaaaactatgggttccggtaagatgttggttgtttctatgggttttaagcaatggaacgatcaattctctgctgaatggtccgatattgaaattgcttgttacaacgctccagattccatagttgttactggtaacgaagaaagattgaaagaattgtccatcaagttgtccgacgaatccaatcaaattttcaacaccttcttgaggtccccatgttcttttcattcttcccatcaagaagtcatcaagggttctatgttcgaagagttgtctaacttgcaatctactggtgaaaccgaaatccctttgttctctactgttactggtagacaagttttgtctggtcatgttactgctcaacacatctacgataatgttagagaaccagtcttgttccaaaagacgattgaatccattacctcctacatcaagtctcactacccatccaatcaaaaggttatctacgttgaaattgctccacacccaaccttgttttcattgatcaaaaagtccatcccatcctccaacaagaattcctcttctgttttgtgtccattgaacagaaaagaaaactccaacaactcctacaagaagttcgtttctcagttgtacttcaacggtgttaacgttgacttcaacttccagttgaactccatttgcgataacgttaacaacgatcaccatttgaacaacgtcaagcaaaactccttcaaagagactaccaattccttgccaagataccaatgggaacaagatgaatattggtccgaaccattgatctccagaaagaatagattggaaggtccaactacttccttgttgggtcatagaattatctacagcttcccagttttccaatccgttttggacttgcaatctgacaactacaaatacttgttggaccacttggttaacggtaagccagtttttccaggtgctggttatttggatatcatcatcgaattcttcgactaccaaaagcagcagttgaattcctctgattcctctaactcctacatcatcaacgttgacaagatccaattcttgaacccaattcacttgaccgaaaacaagttgcaaaccttgcaatcttctttcgaacctatcgttactaagaagtctgccttctctgttaacttcttcatcaaggataccgtcgaggatcaatctaaggttaagtctatgtctgacgaaacttggactaacacttgtaaggctaccatttccttggaacaacaacagccatctccatcttctactttgactttgtctaagaagcaagacttgcagatcttgagaaacagatgcgatattagcaagctagacaagtttgagttgtacgacaagatctctaagaatttgggcttgcagtacaactccttgtttcaagttgttgataccatcgaaactggtaaggattgctcttttgctactttgtctttgccagaagatactttgttcaccaccattttgaacccatgcttgttggataactgtttccatggtttgttgaccttgatcaacgaaaagggttctttcgttgtcgagtccatttcttctgtttctatctacttggagaacatcggttccttcaatcaaacttctgttggtaacgtccagttctacttgtacaccactatttctaaagccacctcctttagttctgaaggtacttgtaagttgttcaccaaggatggttccttgattttgtctatcggtaagttcatcatcaagtccaccaatccaaagtctactaagaccaacgaaactatcgaatctccattggacgaaaccttctctattgaatggcaatctaaggattctccaattccaaccccacaacaaatccaacaacaatctccattgaactctaacccatccttcattagatctaccatcttgaaggacatccagttcgaacaatactgctcctccattatccacaaagaattgatcaaccacgaaaagtacaagaaccagcaatccttcgatatcaactccttggaaaaccacttgaacgatgaccaattgatggaatccttgtccatctccaaagaatacttgagattcttcaccaggatcatctccatcattaagcaatacccaaagatcttgaacgaaaaagagctaaaagaattgaaagaaatcatcgaattgaagtacccatccgaagttcagttgttggaattcgaagttatcgagaaggtgtccatgattatcccaaagttgttgttcgaaaacgacaagcaatcttccatgaccttgttccaagataacttgttgaccaggttctactccaattctaactctaccagattctacttggaaagggtttccgaaatggtcttggaatctattagaccaatcgtcagagaaaagagggtgttcagaattttagagatcggtgctcgtacaggctctttgtctaatgttgttttgactaagttgaacacctacttgtccaccttgaattctaatggtggttctggttacaacatcatcattgagtacaccttcaccgatatttccgccaacttcattattggtgaaatccaagaaaccatgtgcaacttgtacccaaacgttactttcaagttctccgtcttggacttggagaaagagattattaactcctccgatttcttgatgggtgattacgatatagttttgatggcctacgttatccatgccgtttctaacattaagttctccatcgaacagttgtacaagttgttgtctccaagaggttggttgttgtgtattgaacctaagtccaacgttgtgttctccgatttggttttcggttgttttaatcagtggtggaactactacgatgatattagaactacccactgctccttgtctgaatctcaatggaatcagttgttgttgaaccagtccttgaacaacgaatcctcttcttcttctaactgttacggtggtttctccaacgtttcttttattggtggtgaaaaggatgtcgactcccattctttcatattgcactgccaaaaagaatccatctcccaaatgaagttagccaccactattaacaacggtttgtcatctggttccatcgttatcgttttgaactctcaacaattgaccaacatgaagtcctacccaaaggttattgagtatattcaagaggctacctctttgtgcaagaccattgaaattatcgattccaaggacgtcttgaactctaccaattcagttttggaaaagatccaaaagtccttgttggtgttctgtttgttgggttatgacttgttggagaacaactaccaagaacagtctttcgaatacgttaagttgttgaacttgatctctactaccgcctcttcatctaatgataagaaaccaccaaaggtcttgttgatcaccaagcaatctgaaagaatctccaggtctttctactccagatccttgattggtatttccagaacctctatgaacgagtacccaaatttgtccattacctctatcgatttggataccaacgactactcattgcagtctttgttgaagccaatcttcagcaactctaagttttccgacaacgagttcatcttcaaaaagggcttgatgttcgtgtccaggatctttaagaacaagcagttgctagaatcctccaacgcttttgaaactgactcttctaacttgtactgtaaggcctcttctgacttgtcttacaagtacgctattaagcagtctatgttgaccgaaaatcagatcgaaatcaaggttgaatgcgtcggtattaacttcaaggacaacctattctacaagggcttgttgccacaagaaattttcagaatgggtgacatctacaatccaccatatggtttggaatgctctggtgttattaccagaattggttctaacgtcaccgaatactcagttggtcaaaatgtttttggtttcgccagacattctttgggttctcatgttgttaccaacaaggatttggttatcttgaagccagataccatctcattttctgaagctgcttctatcccagttgtttactgtactgcttggtactccttgttcaacattggtcagttgtctaacgaagaatccatcctaattcattctgctactggtggtgtaggtttggcttctttgaatttgttgaaaatgaagaatcagcaacagcaaccattgaccaatgtttatgctactgttggctctaacgagaagaagaagttcttgatcgataacttcaacaacttgttcaaagaggacggcgaaaacattttctctaccagagacaaagaatactccaaccagttggaatccaagatcgatgttattttgaacaccttgtccggtgaattcgtcgaatctaatttcaagtccttgagatccttcggtagattgattgatttgtctgctactcacgtttacgccaatcaacaaattggtctaggtaacttcaagttcgaccacttgtattctgctgttgacttggaaagattgatcgacgaaaaacctaagttgttgcagtccatcttgcaaagaattaccaactctatcgtcaacggttccttggaaaaaattccaattaccatcttcccatccaccgaaactaaggatgctatcgaattattgtccaagagatcccatatcggtaaagttgttgtagattgcaccgatatctctaagtgtaatcctgttggtgatgtgatcaccaacttctctatgagattgccaaagccaaactaccagttgaatttgaactccaccttgttgattactggtcagtctggtttgtctatccctttgttgaattggttgttgtctaagtctggtggtaacgttaagaacgttgtcatcatttctaagtccaccatgaagtggaagttgcagactatgatttcccatttcgtttccggtttcggtatccattttaactacgttcaagtcgacatctccaactacgatgctttgtctgaagctattaagcaattgccatctgatttgccaccaatcacctctgtttttcatttggctgctatctacaacgatgttccaatggatcaagttaccatgtctaccgttgaatctgttcataaccctaaagttttgggtgccgttaacttgcatagaatctctgtttcttttggttggaagttgaaccacttcgtcttgttctcttctattactgctattaccggttacccagaccaatctatctacaattctgccaactctattttggacgctttgtccaactttagaaggtttatgggtttgccatccttctccattaacttgggtccaatgaaggatgaaggtaaggtttctaccaacaagagcatcaagaagctattcaagtctagaggtttgccaagcctatccttgaacaagttatttggtttgttggaggtcgtcatcaacaacccatctaatcatgttatcccatcccaattgatttgctccccaatcgatttcaagacctacatcgaatctttctcaactatgaggccaaagttgttacacttgcaacctaccatttccaagcagcaatcttctatcattaacgattctaccaaggcttcctccaacatttcattgcaagataagatcacctccaaggtgtctgatttgttgtccattccaatctccaagatcaacttcgatcatccattgaaacactacggcttggattctttgttgaccgttcaattcaaatcctggatcgacaaagaattcgaaaagaacttgttcacccatatccaattggccaccatctctattaactcattcttggaaaaggtgaacggcttgtctacaaacaataacaacaacaacaattccaacgtcaagtcctctccatccattgtcaaagaagaaatcgttaccttggacaaggatcaacaaccattgctattgaaagaacaccagcacattatcatctccccagatattagaatcaacaagccaaagagggaatccttgattagaaccccaatcttgaacaaattcaaccagatcaccgaatccattatcactccatctacaccatctttgtcccaatccgatgttttgaaaactccaccaatcaagtctttgaacaacactaagaactccagcttgattaacaccccaccaattcaatctgtccaacaacatcaaaagcaacaacaaaaggtccaagtcatccaacaacagcaacaaccattatccagattgtcctacaagagcaacaacaactctttcgttttgggtatcggtatttctgttccaggtgaacctatttcccaacaatccttgaaagactccatctccaatgacttttctgataaggctgaaactaacgagaaggtcaagagaatctttgagcaatctcaaatcaagaccagacacttggttagagattacactaagccagagaactccatcaagttcagacatttggaaaccattaccgatgtgaacaaccagttcaagaaagttgttccagatttggctcaacaagcctgtttgagagctttgaaagattggggtggtgataagggtgatattacccatatagtttctgttacctccaccggtattatcatcccagatgttaatttcaagttgatcgacttgttgggcttgaacaaggatgttgaaagagtgtctttgaacctaatgggttgtttggctggtttgagttctttgagaactgctgcttctttggctaaggcttctccaagaaatagaattttggttgtctgtaccgaagtctgctccttgcatttttctaatactgatggtggtgatcaaatggtcgcctcttctatttttgctgatggttctgctgcttacattattggttgtaacccaagaattgaagaaaccccattatacgaagtcatgtgctccattaacagatctttcccaaataccgaaaacgccatggtttgggatttggaaaaagaaggttggaacttgggtttggatgcttctattccaattgtcattggttctggtattgaagccttcgttgatactttgttggataaggctaagttgcaaacttccactgctatttctgctaaggattgcgaattcttgattcatactggtggcaagtccatcttgatgaacatcgaaaattccttgggtatcgacccaaagcaaactaagaatacttgggatgtttaccatgcctacggcaatatgtcatctgcctctgttattttcgttatggatcatgccagaaagtccaagtctttgccaacttactcaatttctttggcttttggtccaggtttggcttttgaaggttgtttcttgaagaacgtcgtctgaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtcgaagacatatgcccactttgggaaatagaaatggtcattttaagctcaagtgaagcgaaaaaaggaggtatactcctttacgcgtcacccaatctggataggcacgtcaaactcattaggtctcatcaagaactagtgtagaaatgacgcttgaactccgaactttaaacaaagttctgtttttgtttctgtttctgtttcctgttgctgtctctgtttctatttttttcgtatagctctatttcccttgtagataaacatatataagaatgctattatagaagcgtgtattttcctccccctagcttcaacctgtaattcccttcttagtaaagcgaactagaaccagtttaataggatataga

SEQ ID NO: 78 - DNA, 11637 bp, Artificial Sequence
  DiPKS[G1516R]-5
atgaacgctaatatatgggtggctgcttcagatggtaatttggaccgagtggaacatatcctccgcgagagtaaaggcgccatgaccccgcaatccaaggacattaacggctacactccaatgcatgctgccgccgcatacggccacctggatttgctgaagaaaatgtgcaatgagtacaatggagacattaatgtgttggacaacgacggcgataccccgttgcaccatgtggaggatgtggccactgccaggttgatcgtggaagagctgggtggagacttcactatcaggaatgtggagggccaaacgccatacgactcgttcgtcgagaacggtgaagatggtgagctaatcgagtacatgaggattaagtccggcgtggccgatgttcacggagtggacggcgtgcagggtgagggtgtcatcgacagcaaattgctggaagagttcaaggacaacgtgagatacaccttggaaaatgaccctgaggaaggagccgatgaggccactctgcaacgcaggaggcagttggaacagatcattacgggagacaacgctgaggaggagttggaaaggtacatccgtgctatggtcagagagcagatgctgggccagggctccatggcgggttccggggacgaaccagattccaagagaagaaaataacgaaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaataaacaatgaacaagaactccaaaatccagtccccaaactcttctgatgttgctgttattggtgttggttttagattcccaggtaactctaatgacccagaatctttgtggaacaacttgttggatggtttcgatgctattacccaagtcccaaaagaaagatgggctacttcttttagagagatgggtttgatcaagaacaagttcggtggtttcttgaaggattctgaatggaagaatttcgaccctttgttctttggtatcggtccaaaagaagctccattcattgatccacaacaaaggttgttgttgtccatcgtttgggaatctttggaagatgcttacatcagaccagatgaattgagaggttctaacactggtgttttcatcggtgtttctaacaacgattacaccaagttgggtttccaagacaactactctatttctccatacactatgaccggctctaactcttcattgaactccaacagaatttcctactgcttcgattttagaggtccatccattactgttgataccgcttgttcttcttccttggtttctgttaatttgggtgtccaatccatccaaatgggtgaatgtaagattgctatttgcggtggtgttaacgctttgtttgatccatctacatctgttgccttttccaagttgggtgttttgtctgaaaatggcagatgcaactcttttagtgatcaagcctctggttacgttagatctgaaggtgctggtgttgttgttttgaagtctttggaacaagctaagttggatggtgatagaatctacggtgttatcaagggtgtttcctctaatgaagatggtgcttctaatggtgacaagaactctttgactactccatcttgtgaagcccaatccattaacatttctaaggctatggaaaaggcctccttgtctccatctgatatctattacattgaagcccatggtactggtactccagttggtgatccaattgaagttaaggccttgtccaagatcttctccaactctaacaacaaccagttgaacaacttctctaccgatggtaatgataacgatgatgatgatgacgataacacctctccagaaccattattgattggctcattcaagtccaacatcggtcatttggaatctgctgctggtattgcttctttgattaagtgttgcttgatgttgaagaacaggatgttggttccatccattaactgctctaatttgaacccatccattccattcgatcagtacaacatctccgttatcagagaaatcagacaattcccaaccgataagttggttaacatcggtatcaattctttcggtttcggtggttctaactgccatttgattattcaagagtacaacaacaacttcaagaacaactctaccatctgcaataacaacaacaacaacaataacaacatcgactacttgatcccaatctcctctaagactaagaagtccttggataagtacttgattttgatcaagaccaactccaactaccacaaggatatttctttcgatgacttcgtcaagttccaaatcaagtctaagcagtacaacttgtccaacagaatgactaccattgctaacgattggaactccttcattaagggttctaacgaattccacaacttgatcgaatctaaggatggtgaaggtggttcttcatcttctaacagaggtattgattccgccaatcaaatcaacactactactacctctaccatcaacgatatcgaacctttgttggttttcgttttctgtggtcaaggtccacaatggaatggtatgattaagaccttgtacaactccgagaacgttttcaagaacaccgttgatcatgttgacagcatcttgtacaagtacttcggttactccattttgaacgtcttgtctaagatcgatgataacgacgattccatcaaccatccaatagttgctcaaccatctttgttcttgttgcaaattggtttggtcgagttgtttaagtactggggtatctacccatctatctctgttggtcattctttcggtgaagtctcttcttattacttgtccggtatcatctctttggaaaccgcttgtaaaatcgtctacgtcagatcctctaatcagaacaaaactatgggttccggtaagatgttggttgtttctatgggttttaagcaatggaacgatcaattctctgctgaatggtccgatattgaaattgcttgttacaacgctccagattccatagttgttactggtaacgaagaaagattgaaagaattgtccatcaagttgtccgacgaatccaatcaaattttcaacaccttcttgaggtccccatgttcttttcattcttcccatcaagaagtcatcaagggttctatgttcgaagagttgtctaacttgcaatctactggtgaaaccgaaatccctttgttctctactgttactggtagacaagttttgtctggtcatgttactgctcaacacatctacgataatgttagagaaccagtcttgttccaaaagacgattgaatccattacctcctacatcaagtctcactacccatccaatcaaaaggttatctacgttgaaattgctccacacccaaccttgttttcattgatcaaaaagtccatcccatcctccaacaagaattcctcttctgttttgtgtccattgaacagaaaagaaaactccaacaactcctacaagaagttcgtttctcagttgtacttcaacggtgttaacgttgacttcaacttccagttgaactccatttgcgataacgttaacaacgatcaccatttgaacaacgtcaagcaaaactccttcaaagagactaccaattccttgccaagataccaatgggaacaagatgaatattggtccgaaccattgatctccagaaagaatagattggaaggtccaactacttccttgttgggtcatagaattatctacagcttcccagttttccaatccgttttggacttgcaatctgacaactacaaatacttgttggaccacttggttaacggtaagccagtttttccaggtgctggttatttggatatcatcatcgaattcttcgactaccaaaagcagcagttgaattcctctgattcctctaactcctacatcatcaacgttgacaagatccaattcttgaacccaattcacttgaccgaaaacaagttgcaaaccttgcaatcttctttcgaacctatcgttactaagaagtctgccttctctgttaacttcttcatcaaggataccgtcgaggatcaatctaaggttaagtctatgtctgacgaaacttggactaacacttgtaaggctaccatttccttggaacaacaacagccatctccatcttctactttgactttgtctaagaagcaagacttgcagatcttgagaaacagatgcgatattagcaagctagacaagtttgagttgtacgacaagatctctaagaatttgggcttgcagtacaactccttgtttcaagttgttgataccatcgaaactggtaaggattgctcttttgctactttgtctttgccagaagatactttgttcaccaccattttgaacccatgcttgttggataactgtttccatggtttgttgaccttgatcaacgaaaagggttctttcgttgtcgagtccatttcttctgtttctatctacttggagaacatcggttccttcaatcaaacttctgttggtaacgtccagttctacttgtacaccactatttctaaagccacctcctttagttctgaaggtacttgtaagttgttcaccaaggatggttccttgattttgtctatcggtaagttcatcatcaagtccaccaatccaaagtctactaagaccaacgaaactatcgaatctccattggacgaaaccttctctattgaatggcaatctaaggattctccaattccaaccccacaacaaatccaacaacaatctccattgaactctaacccatccttcattagatctaccatcttgaaggacatccagttcgaacaatactgctcctccattatccacaaagaattgatcaaccacgaaaagtacaagaaccagcaatccttcgatatcaactccttggaaaaccacttgaacgatgaccaattgatggaatccttgtccatctccaaagaatacttgagattcttcaccaggatcatctccatcattaagcaatacccaaagatcttgaacgaaaaagagctaaaagaattgaaagaaatcatcgaattgaagtacccatccgaagttcagttgttggaattcgaagttatcgagaaggtgtccatgattatcccaaagttgttgttcgaaaacgacaagcaatcttccatgaccttgttccaagataacttgttgaccaggttctactccaattctaactctaccagattctacttggaaagggtttccgaaatggtcttggaatctattagaccaatcgtcagagaaaagagggtgttcagaattttagagatcggtgctcgtacaggctctttgtctaatgttgttttgactaagttgaacacctacttgtccaccttgaattctaatggtggttctggttacaacatcatcattgagtacaccttcaccgatatttccgccaacttcattattggtgaaatccaagaaaccatgtgcaacttgtacccaaacgttactttcaagttctccgtcttggacttggagaaagagattattaactcctccgatttcttgatgggtgattacgatatagttttgatggcctacgttatccatgccgtttctaacattaagttctccatcgaacagttgtacaagttgttgtctccaagaggttggttgttgtgtattgaacctaagtccaacgttgtgttctccgatttggttttcggttgttttaatcagtggtggaactactacgatgatattagaactacccactgctccttgtctgaatctcaatggaatcagttgttgttgaaccagtccttgaacaacgaatcctcttcttcttctaactgttacggtggtttctccaacgtttcttttattggtggtgaaaaggatgtcgactcccattctttcatattgcactgccaaaaagaatccatctcccaaatgaagttagccaccactattaacaacggtttgtcatctggttccatcgttatcgttttgaactctcaacaattgaccaacatgaagtcctacccaaaggttattgagtatattcaagaggctacctctttgtgcaagaccattgaaattatcgattccaaggacgtcttgaactctaccaattcagttttggaaaagatccaaaagtccttgttggtgttctgtttgttgggttatgacttgttggagaacaactaccaagaacagtctttcgaatacgttaagttgttgaacttgatctctactaccgcctcttcatctaatgataagaaaccaccaaaggtcttgttgatcaccaagcaatctgaaagaatctccaggtctttctactccagatccttgattggtatttccagaacctctatgaacgagtacccaaatttgtccattacctctatcgatttggataccaacgactactcattgcagtctttgttgaagccaatcttcagcaactctaagttttccgacaacgagttcatcttcaaaaagggcttgatgttcgtgtccaggatctttaagaacaagcagttgctagaatcctccaacgcttttgaaactgactcttctaacttgtactgtaaggcctcttctgacttgtcttacaagtacgctattaagcagtctatgttgaccgaaaatcagatcgaaatcaaggttgaatgcgtcggtattaacttcaaggacaacctattctacaagggcttgttgccacaagaaattttcagaatgggtgacatctacaatccaccatatggtttggaatgctctggtgttattaccagaattggttctaacgtcaccgaatactcagttggtcaaaatgtttttggtttcgccagacattctttgggttctcatgttgttaccaacaaggatttggttatcttgaagccagataccatctcattttctgaagctgcttctatcccagttgtttactgtactgcttggtactccttgttcaacattggtcagttgtctaacgaagaatccatcctaattcattctgctactggtggtgtaggtttggcttctttgaatttgttgaaaatgaagaatcagcaacagcaaccattgaccaatgtttatgctactgttggctctaacgagaagaagaagttcttgatcgataacttcaacaacttgttcaaagaggacggcgaaaacattttctctaccagagacaaagaatactccaaccagttggaatccaagatcgatgttattttgaacaccttgtccggtgaattcgtcgaatctaatttcaagtccttgagatccttcggtagattgattgatttgtctgctactcacgtttacgccaatcaacaaattggtctaggtaacttcaagttcgaccacttgtattctgctgttgacttggaaagattgatcgacgaaaaacctaagttgttgcagtccatcttgcaaagaattaccaactctatcgtcaacggttccttggaaaaaattccaattaccatcttcccatccaccgaaactaaggatgctatcgaattattgtccaagagatcccatatcggtaaagttgttgtagattgcaccgatatctctaagtgtaatcctgttggtgatgtgatcaccaacttctctatgagattgccaaagccaaactaccagttgaatttgaactccaccttgttgattactggtcagtctggtttgtctatccctttgttgaattggttgttgtctaagtctggtggtaacgttaagaacgttgtcatcatttctaagtccaccatgaagtggaagttgcagactatgatttcccatttcgtttccggtttcggtatccattttaactacgttcaagtcgacatctccaactacgatgctttgtctgaagctattaagcaattgccatctgatttgccaccaatcacctctgtttttcatttggctgctatctacaacgatgttccaatggatcaagttaccatgtctaccgttgaatctgttcataaccctaaagttttgggtgccgttaacttgcatagaatctctgtttcttttggttggaagttgaaccacttcgtcttgttctcttctattactgctattaccggttacccagaccaatctatctacaattctgccaactctattttggacgctttgtccaactttagaaggtttatgggtttgccatccttctccattaacttgggtccaatgaaggatgaaggtaaggtttctaccaacaagagcatcaagaagctattcaagtctagaggtttgccaagcctatccttgaacaagttatttggtttgttggaggtcgtcatcaacaacccatctaatcatgttatcccatcccaattgatttgctccccaatcgatttcaagacctacatcgaatctttctcaactatgaggccaaagttgttacacttgcaacctaccatttccaagcagcaatcttctatcattaacgattctaccaaggcttcctccaacatttcattgcaagataagatcacctccaaggtgtctgatttgttgtccattccaatctccaagatcaacttcgatcatccattgaaacactacggcttggattctttgttgaccgttcaattcaaatcctggatcgacaaagaattcgaaaagaacttgttcacccatatccaattggccaccatctctattaactcattcttggaaaaggtgaacggcttgtctacaaacaataacaacaacaacaattccaacgtcaagtcctctccatccattgtcaaagaagaaatcgttaccttggacaaggatcaacaaccattgctattgaaagaacaccagcacattatcatctccccagatattagaatcaacaagccaaagagggaatccttgattagaaccccaatcttgaacaaattcaaccagatcaccgaatccattatcactccatctacaccatctttgtcccaatccgatgttttgaaaactccaccaatcaagtctttgaacaacactaagaactccagcttgattaacaccccaccaattcaatctgtccaacaacatcaaaagcaacaacaaaaggtccaagtcatccaacaacagcaacaaccattatccagattgtcctacaagagcaacaacaactctttcgttttgggtatcggtatttctgttccaggtgaacctatttcccaacaatccttgaaagactccatctccaatgacttttctgataaggctgaaactaacgagaaggtcaagagaatctttgagcaatctcaaatcaagaccagacacttggttagagattacactaagccagagaactccatcaagttcagacatttggaaaccattaccgatgtgaacaaccagttcaagaaagttgttccagatttggctcaacaagcctgtttgagagctttgaaagattggggtggtgataagggtgatattacccatatagtttctgttacctccaccggtattatcatcccagatgttaatttcaagttgatcgacttgttgggcttgaacaaggatgttgaaagagtgtctttgaacctaatgggttgtttggctggtttgagttctttgagaactgctgcttctttggctaaggcttctccaagaaatagaattttggttgtctgtaccgaagtctgctccttgcatttttctaatactgatggtggtgatcaaatggtcgcctcttctatttttgctgatggttctgctgcttacattattggttgtaacccaagaattgaagaaaccccattatacgaagtcatgtgctccattaacagatctttcccaaataccgaaaacgccatggtttgggatttggaaaaagaaggttggaacttgggtttggatgcttctattccaattgtcattggttctggtattgaagccttcgttgatactttgttggataaggctaagttgcaaacttccactgctatttctgctaaggattgcgaattcttgattcatactggtggcaagtccatcttgatgaacatcgaaaattccttgggtatcgacccaaagcaaactaagaatacttgggatgtttaccatgcctacggcaatatgtcatctgcctctgttattttcgttatggatcatgccagaaagtccaagtctttgccaacttactcaatttctttggcttttggtccaggtttggcttttgaaggttgtttcttgaagaacgtcgtctgaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtctaatagacgttcttacaaggtaaaatttcaccgcgtttttaaatagaatgaaaaaaacgttgtagagtgaaagaaaagcaacaaatatacagttcacaaggcagcttcgtatagtaatacagcacgaaaaacagctcatagaaatggtaacacagaccaatccggtccctgttacatatccaacggatgcttatatccccacgtatctgcccgatgataaggtctccaatctggcagatttgaaaaaattgatagaaatggattccagactagatttgtatctgacaagaaggaggctggatacgtccatcaatttacctacaaacaccaagaccaaggaccatccccccaataaagagatgctgaggatttacgtctacaacactacggaaagcagccctcgcagcgattctggcaccccagcggactcaggcaagactacatggacactgagaatagaaggtaagcttctgcacgagtccgc

SEQ ID NO: 79 - DNA, 7114 bp, Artificial Sequence
  PDH
caccggagcttggatatgataaacgaaatattcttgaatcgtgagatcgcctgttttcaaaaccgttggaggcagaaacaattttgtcacaagatgggcattctaccccatccttgctgtattattgtagtctcgctttcttttatgctggacaaatgagactactgcacatttttatacgttcttggttttttttaaaggtgtggtttcggcattatcctgccgcacgtttcttggataattcatcctgattctctattttaaacgcttcagcctatcaggatttggttttgatacatactgcaagagtgtatctcgggaacagtcatttattccgcaacaaacttaattgcggaacgcgttaggcgatttctagcatatatcaaataccgttcgcgatttcttctgggttcgtctcttttcttttaaatacttattaacgtactcaaacaactacacttcgttgtatctcagaatgagatccctcagtatgacaatacatcattctaaacgttcgtaaaacacatatgaaacaactttataacaaagcgaacaaaatgggcaacatgagatgaaactccgcgtcccttagctgaactacccaaacgtacgaatgcctgaacaattagtttagatccgagattccgcgcttccatcatttagtataatccatattttatataatatataggataagtaacagcccgcgaaaaacaacaaataatcataaaaattttagaactagacatatcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgactagaaaatttattataaaaggaagagaaataattaaacaatgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctatgggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaaagacataaaactgaaacaacaccaattaataatagacttttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatggcttaaataacatactcatcactaaacattcttaacaatcaaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaatacatctaccagtcaacagccaacaattaactaattaaacaatgtcccaaactcataagcacgctattccagctaatattgctgatagatgcttgatcaacccagaacagtacgaaactaagtacaagcaatccatcaacgatccagatactttttggggtgaacaaggtaagattttggattggattaccccataccaaaaggtcaagaatacttcttttgctccaggcaacgtttccattaagtggtatgaagatggtactttgaacttggctgctaactgtttggatagacacttgcaagaaaacggtgatagaaccgctattatttgggaaggtgatgatacctcccaatccaaacatatctcttacagagaattgcacagagatgtctgtagattcgctaacactttgttggatttgggcatcaaaaagggtgatgttgttgctatctatatgccaatggttcctgaagctgctgttgctatgttggcttgtgctagaattggtgctgttcattctgttattttcggtggtttttcaccagaagctgttgccggtagaattatcgattcttcatccagattggttatcaccgctgatgaaggtgttagagctggtagatctattccattgaaaaagaacgttgatgacgccttgaagaacccaaatgttacttctgttgaacacgtcatcgttttgaagagaactggttctgatatcgattggcaagagggtagagatttgtggtggagagatttgattgaaaaggcttctccagaacatcaaccagaagctatgaacgctgaagatcctttgtttatcttgtacacttctggttctactggtaagccaaaaggtgttttacacactactggtggttatttggtttacgctgctactactttcaagtacgttttcgattatcacccaggtgatatctattggtgtactgctgatgttggttgggttactggtcattcttatttgttgtatggtccattggcttgtggtgctactacattgatgtttgaaggtgttccaaattggccaactccagctagaatgtgtcaagttgttgacaaacaccaagtcaacatcttgtatactgctccaactgctattagagctttgatggctgaaggtgataaggctattgaaggtactgatagatcctccttgagaatcttgggttctgttggtgaacctattaaccctgaagcctgggaatggtattggaagaaaattggtaaagaaaagtgcccagttgttgatacttggtggcaaactgaaactggtggttttatgattactccattgccaggtgctattgaattgaaagctggttctgctactagaccattttttggtgttcaaccagctttggttgataacgaaggtcatccacaagaaggtgctactgaaggtaatttggttattactgattcttggccaggtcaagctagaactttgtttggtgatcacgaaagattcgaacagacttacttctctaccttcaagaacatgtacttctctggtgatggtgctagaagagatgaagatggttactattggattaccggtagagttgatgatgtcttgaatgtttctggtcacagattaggtactgccgaaattgaatctgctttggttgctcatccaaagattgctgaagctgcagttgttggtattccacatgctattaagggtcaagctatctacgcttacgttactttgaatcatggtgaagaaccatctccagaattatacgctgaagttagaaactgggtcagaaaagaaattggtccattagctaccccagatgttttacattggactgattctttgccaaagaccagatcaggtaagatcatgagaagaatcttgagaaagattgctgctggtgatacttctaacttgggtgatacttcaacattagctgatccaggtgttgttgaaaagcctttggaagaaaaacaagctattgccatgccatcctaataattaaatactattttcaaaattctacttaaaaataacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtcattgcgaagactatactgatatatgaatttaaactagagcggaccaactatcatccgctaattactgacattaccaaatgagatctgtgaatgggcaagataaaaaacaaaaattgaaatgtttgacgttatgtaaaactattaattccttcgctttcggcggtcacagaatttgcgtgtagctgactcttgttcaatcaatatcatttgttactttatttgaaagtctgtattactgcgcctattgtcatccgtaccaaagaacgtcaaaaagaaacaagataatttttgtgcttacaccatttatagatcactgagcccagaatatcgctggagctcagtgtaagtggcatgaacacaactctgactgatcgcacatattgccgttatcataaatactagttgtacttgtcaatgcgacgaatggcatcatgcctattattacgttcctctttttccgtttcatgtttccagaatgctattgaatctaacacttcaattataaaaaagaataaatccgcaataattttaggctaattgttgtactgtcaagcgaacctaatggttaaaattcagaggaaccttcgacgtagtctgatcgctacttctatatcttatgttcccagtcaatcaaaagttgatactataatagctgccatttatacctgttagttatggcgatcgtttatcacg

SEQ ID NO: 80 - DNA, 3256 bp, Artificial Sequence
  Maf1
aatgatttaagcgtgcgtgaagataacactacaatccattttaaagcaacatccacattgagtgtatacaccacaaaggttttttcagggcgtttttctcgccactttatgttgaccaaaattattaatggaacttacaacgtttccaaaagttagttaaatacatacgtctatttactaagcaagaaatatatcatgacaagcccaaatattatattgttatgtttacaaaaaaaaaatggctatatacatcaagtctggaggctttttataacaagcaagtggggtaacttagacataagattgacttctttgaattcaacaaaaatacatacttttgatgatttcaatggtagaagcataaacaacaaataatcataaaaattttagaactagacataaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaatctagaaaatttattataaaaggaagagaaataattaaacaatgaaatttattgatgagctagatatagagagagtgaatcaaactctcaatttcgagacaaatgactgtaaaatcgtgggcagttgcgatattttcacaacaaaggcggttgcatcagatagaaaattatataaaactattgatcagcatttggatactattttacaggaaaatgagaattacaatgctacccttcagcaacagctagctgctcccgaaacaaaccaatcaccctgctcgtcgccattttattctaataggagggatagcaactctttttgggagcaaaagagaagaatatcttttagtgaatacaatagcaataataacactaacaacagtaatggcaatagcagtaataacaataactattctggacctaatggttcttctccagcaacttttcccaaaagtgccaagctaaatgaccaaaatttaaaagaattagtctcgaattacgattctggctctatgagctcatcgtctcttgattcttcttctaagaatgatgagaggataagaagaaggagcagtagcagtattagcagtttcaaaagtggtaaatcatcgaacaataattacagttctggtacagcaaccaacaatgttaacaaaagaagaaaatcttcgataaacgaaaggccaagcaatttaagtttgggtccgtttggtcccataaacgaaccgtcaagccgcaaaatatttgcttatctgattgctatcctcaacgcttcttatcctgaccatgatttttcatcggttgagccaacggattttgtcaaaacatcattgaaaacttttatttccaaatttgaaaacaccttatattctcttggtagacaaccagaggaatgggtctgggaggtaattaattctcacatgactctttctgattgcgtcctttttcaatattcaccttcaaactcttttttggaagatgagcctggctatctttggaatcttataggttttctttacaacaggaaaaggaaaagagtggcttacctttacttgatttgctcgcgtctaaattcgagtacaggcgaagtggaagatgccttggcaaaaaaacctcagggaaagcttataatagatgatggctcaaatgaatacgaaggagaatacgatttcacttatgatgagaatgtaatagatgataaatcagatcaagaagaatccctacagtagagacataaaactgaaacaacaccaattaataatagactttacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtccttaaataacatactcatcactaaacattcttaacaatcagaaaacaacgcgtcatgaaaaagagttactgaaccttcagatcctacttattgtaatgcttcgcgacatccaatccatttaataatcaatttaaaactagagttggtagagttccttgttgaacgtgataacccaaaagcataatacgagtaatgtttcagtattgctattatatgtttacacaaggaaaacatataataacaaacctctaatccggtagtacttaagaaactatagtttctatgtacaaaaaggtaactatgtaattcttacatttacataacatatagaagggtccaataaacttactaaacttactaccttgttgtatataggctagatcgtaatccactacgtcaacataaaaaaaacttaaggagtttgaattttatgtacaaacagattgttaaaatataatataagattatggaaacgaacttgctctaaaaaaaatttaaagttttataaaatcctcgcactatcgctgttatacatgatgtccccaaagcgtgtac

SEQ ID NO: 81 - DNA, 4538 bp, Artificial Sequence
  Erg20K197E
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacgcatctacgactgtgggtcccgtggagaaatgtatgaaaccctgtatggagagtgattagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatcatacgttgaaactacggcaaaggattggtcagatcgcttcatacaggggtatcccggcataaccctcactaaagggaacaaaagctggagctcgtttaaaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaatggcgggggtcggaatgattaaagaaaggggctgtgggcgagattgaaacaatggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcgagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaataaccagttaataaaccgtggcaaacatgatggtggcctaatggaggtcaccaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtcgcggccgcggtacctaataacttcgtatagcatacattatacgaagttat

SEQ ID NO: 82 - DNA, 3503 bp, Artificial Sequence
  Erg1pUB14-Erg20deg
tgtgcacaaaggccataatattatgtctacagaatatactagatgtcctccctataggatatagtaatcctctaaatggaaccgatatttctacataataatattacgattattcctccttccgttttatatgtttcattatcctagcacactatcaatctttgcatttcagcttccattagatttgatgactatttctcaatctttatgttatctccttacgccgcatgtgataatatactgctagtatgactactagttgatagaagatagttgatttttactccaacaaaagtaacaatattatttagaactatagattccattttgtgcattcccatattctcgaggaaaacttttagtatattctgtagacataatattatcgcctttgtgaacaatagaatcccaacaattgtcgcaaatttaccaattttctagattgcagtcaccttttcaattaatcactagtgtttcacttgtaacattgtcgttgttgtttaacgtattctgtcccgtgccaactatgacaaaaatgcaatgatttcagcggttaaatacgaagcgcaacaagagttagcgaaaaataagtaccaccattctacgctaccattacttactgaaattagagacaactgttatctattggcagatgttcatacggggctttcaaatattgatgaaattatgtgatgtttagaagaagattcgaactgttttcagtagatttggtaactgtgcaaccataactcatgcctacgttcgggatttaatcttctcgcagtctgcaggcgccttgagatttgcgttcggcctaaacgtttgctccacaaacgtgaatggtatgaacatggacatgagcgtggttcagggcactctacgggatcgtggcgaatgggaatcgttctgcaagctcttctaccaaaccatcggcgaatttgcgtcgctttaatgcgatactgccgtagcgggccttcgtatagctcggccgagctcgtacaaaaggcaagcagtgtatcggacagagctgatataacacaatacgctcgtagtcgatgcatgccgtggctgctctcggtcgggtataagtcttagacaatagtcttacctcgcatgtataataaatcttttgtatttaatctattatatgtttctatgcttttttttcctattgttgtttgcttttccttttccttatttctttctagcttctaattttctttcttttttttttttttttcattgaaaattatatatatatatatatatcagaacaattgtccagtattgaacaatacaggttatttcgaacaattgaaaaaaaaaaatcacagaaaaacatatcgagaaaagggtcaaaacaatgcagattttcgtcaagactttgaccggtaaaaccataacattggaagttgaatcttccgataccatcgacaacgttaagtcgaaaattcaagacaaggaaggtatccctccagatcaacaaagattgatctttgccggtaagcagctagaagacggtagaacgctgtctgattacaacattcagaaggagtccaccttacatcttgtgctaaggctaagaggtggtttccacaaatctggtgcttggttgttgccagtttctttggttaagggttctggtgcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatagtggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatggaatcgtccccaacaaaagtgggctctcaaaattcatcacatttaaatgcatataggaagagcaacagttggtttgcatctgatgttccttaaagatttcgacataatgtgcgaagtagataaaatgggtcatttattaatagttatttcattattaaccagttgtggtacaaatgcaactaaagaaaaaaactactaaactatccgggaaatgcgccttagattgcacttcttaattcttattttcgatttttatttttcctttgataatcataaagagaaacgacgatcatttctaaagccatttctgctagtataccgttaaataagaaaaataaagccaaatattataatttttctaatgtgaatccataaatatcaaagcatgcaaaaagggaaagaagtaatgtcttggatttatatagcgtatttgtctaaggggagccagctttggcgcgcctatcgagagaccgattaca

SEQ ID NO: 83 - DNA, 4859 bp, Artificial Sequence
  tHMGr-IDI
ggcgcgcccgagatctttgtgttcggttacccggctcagatcctaacttcgtatgtttattcgtataagttactgttgtccacaggcaatactctgcagaaaattaaaacggcattaatgctaggacaaccagaattgttactactgtatgtgcgatagttgataactgcaacattatgcccggtatattctcaaaaaaccctattactgcatacgaagaaatcgctcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaaaacaatggttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaatggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaataaacaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaacagaagacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaatgcctctagagatgaaaaacaatcgtaaaagggtcctgcgtaattgaaacatttgatcagtatgcagtggcacagaaacaaccaggaatactatagtcataggcaatacaaggtatatattggctatgcagacccctccagaaagtaccgacgtcaagttagatacacttaacgaacctagtgcacatttaattgagaaaaatgtggctcttcctaaggacatattccgttcgtacttgagttattggatctatgaaatcgctcgctatacaccagtcatgattttgtcacactcgtatatgcatgttgttgaaactctgttacgctgaactaacaatcacacatgtagaggtcaccgggaaaagttgcgaccccatggaaggtcgatctcttcgtttggctttgcttggctggcggcattgcgcttcttcgcttatacccgtctcttgacgctcgagctcgttcattgagatacctttattcttgcacattttctggcttttttcgctactcgggtacggcgcgcc

SEQ ID NO: 84 - DNA, 7673 bp, Artificial Sequence
  PGK1pACC1S659A,S1157A
caagtcgcagtcgaaattcaaccgctcattgccactctctctactgcttggtgaactaggctatacgctcaatcagcgccaagatatataagaagaacagcactcccagtcgtattctggcacagtatagcctagcacaatcactgtcacaattgttatcggttctacaattgttctgctctcttcaattttcctttccttattctactctttttatccctacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 85 - DNA, 2177 bp, Artificial Sequence
  OAC
ttagtagatgatagttgatttttattccaacattattgtactcagcaattgttgcgaaacagacaagattaagtatattattcaaggccttgcttacctagatgaaccattgagaaatatacatattgagcaatacctctaggtccgaagttccgtgcctacaccctttagtcagatttagattatatgagataagtggtattttgtggaccaagtttgtgattaatctgatatcaagttactttcttaacgacattaggacgccagggtagccagtattattcaagtccatagaacagcctgtataccttcacttcattaggtatattcttggttatgcgttatttaaatcctcatctgccgctgcttaaaaaaagcagctaaagtgttgcgtaggcacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagatggccgtcaagcacttgatcgtcttaaagtttaaggacgagatcactgaagcccaaaaggaagaattcttcaaaacttacgttaacttggttaacatcattcctgctatgaaggacgtctactggggtaaggatgtcactcaaaaaaacaaggaagaaggttatacccacatcgttgaagtcaccttcgaatctgttgaaactattcaagactacatcattcacccagctcacgtcggtttcggtgatgtttacagatctttctgggaaaaattgttgatcttcgactacaccccaagaaagggctaaagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataactacatggatgataagaaaacatggagtacagtcactttgagaaccttcaatcagctggtaacgtcttcgttaattggatactcaaaaaagatggatagcatgaatcacaagatggaaggaaatgcgggccacgaccacagtgatatgcatatgggagatggagatgataccttccgttaattcgggtttcaatcacttcgtttgtctatcgtatcgcagcctagtgaatatttaattctttcaataaaaaaggcttaaaatcaccatgaaaatcacaagaggaatctttcaacaagaacaatagtatacaatccatgaatgaaggagttcatatttgactagaactatacaaaggttttgtactgtttttactgtgagggtagcatctcatttatccagaatctcagaagggagaaacccaatcgctaaatcgatattatcacccagaagtttcaaataggcgattgtagaatatacacagggacaaccactcagtttatggcttcagtgcgcgcccagttctggaggaaagcattatcgaagtaataaaactttaaaaaagctctccgcaactaatgaggattgcagtatccagatacccttgttttctgccaaatgaacacttttactatgtcatataaacaaaaagtgccatttcttggtactgcttcagtcttaggaatggtcaatacgttattattacttaaatatacgattaataacatgctctgttgcgtatttagtaagtgtgtaaggaaactgtaatattacctaaccggtatcccacgtgtttcacgaa

SEQ ID NO: 86 - DNA, 3600 bp, Artificial Sequence
  PT254
agtatgacaagttttaatcgagatagtttgacgttcgttttttactttgaatatactcgtagtctttttactttttgagtataaaaaaaaaatgactagcaaaataaaattagtagtctaaaaaagaaagctcgcactcaggatcgaactaaggaccaacagatttgcaatctgctgcgctaccactgcgccatacgagcttttgaattatggtaattttgattatcctagaatgttatatctcaatatctcaatatattttggacatctatgaaacacccataaagcagccgctaccaaacagacaagattcagtatgtaaggtaaatacctttttgcacagttaaactacccaaacttattaaagcttgataaattactgaaattccacctttcagttagattcaggcctcatatagattagatatagggtacgtaacattctgtcaaccaagttgttggaatgaaagtctaaaatgtcatctattcggtagcactcatgttactagtatactgtcacatgcggtgtaacgtggggacataaaacagacatcaaatataatggaagctgaaatgcaaagatcgataatgtaataggaatgaaacatataaaacgaaaggagaagtaatggtaatattagtatgtagaaataccgattcaattttggggattcttatattctcgagagaatttctagtataatctgtatacataatattataggctttaccaacaatggaatttcgacaattatcatattattcaccaattaatcacaagttggtaatgagtttgataacaagttactttcttaacaacgttagtatcgtcaaaacactcggttttactcgagcttgtagcacaataataccgtgtagagttctgtattgttcttcttagtgcttgtatatgctcatcccgaccttccattcggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaaacaatgagagccggttccgatcaaattgaaggttctccacatcatgaatccgataactctattgccaccaagattttgaacttcggtcatacttgttggaagttgcaaagaccatatgttgtcaagggtatgatttctattgcttgcggtttgtttggtcgtgagttgtttaacaacagacacttgttttcttggggcttgatgtggaaagctttttttgctttggttcccatcctgtcctttaatttctttgccgccattatgaaccagatctacgatgttgatatcgacagaatcaacaagccagatttgccattggtttctggtgaaatgtctattgaaaccgcctggattttgtccattatcgttgctttgactggtttgatcgttaccatcaagttgaaatctgctcccttgttcgttttcatctacatctttggtattttcgctggtttcgcttattccgttccaccaattagatggaaacaatacccattcaccaatttcttgatcaccatctcttctcatgttggtttggctttcacttcttactctgctactacttctgctttgggtttaccatttgtttggaggccagctttctctttcattattgctttcatgaccgttatgggtatgaccattgctttcgctaaggatatctctgatattgaaggtgatgctaagtacggtgtttctactgttgctacaaaattgggtgctagaaacatgacctttgttgtctctggtgttctgttgttgaactacttggtttccatttccattggtatcatttggccacaggttttcaagtccaacatcatgattttgtctcatgccatcttggctttctgcttgatttttcaaactagagaattggccttggctaactatgcttctgctccatctagacaattcttcgaattcatctggttgttgtactacgccgaatacttcgtctacgtgttcatttgactcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgctttgcgaaaccctatgctctgttgttcggatttgaaattttaaaactacattaatgtgttagtttttctttctttctttctttgtcttgacgtgatttggacttctgtcttgcattcgcgtccattcatctgacccaatattccttttggttttgttatccttataaaaagaaaggaagcttcttagagggaaaaaaatgatgaagagtaatgccaaaatataaataaataaataaatatgaaaatcattttctatttttaatagaataagaagagcatcttaagattacaatttcaagaaatagtttacacagtatatccaataactccaataaactactttcctatacaaatttctatggtgggattaatagtaaaacttctgtacttctctaattcaccaagaaattaaggtaaacatctggtaagcactatccagctttttgctattacacatatggcttttctgcaatcatttcttcccattttgtctcaagccgttagtcttgaaaccacaggcggagtagagttacttgatgcggtattttacatgccttttttcactgcaaaaaaaatgaaatacatatttacacgatttgcaggacagtttacgatagtgagtatgcagaatagttaacacctttgttttatccttttgtgtcttaattatatgatataaaggcgcctggcgttatcggatagtaatagatgctagttatcaacatttcacaattgaaggaaataaagttgaagtactcaacaaaaacttacttcagaattaaatttttggggggaacataggcatcctatgacaggtgaccacaagcccctcaacgcaatctaatattttacaaagtggtaaaattctttcgttcttcgttttaatatacagtcatttattgattctattac

SEQ ID NO: 87 - DNA, 4684 bp, Artificial Sequence
  OstI-pro-alpha-f(I)-OXC53
gcagagaaggagcaaataagccagttttaaggaaccgagataagctgttttcgcccattgttctagttctacaaaatatctacagggaatcgatgaggttgtaagaaatcctgaatactctatgatagtgcataacactaaaaagttgaaagaatcccgtatcatggacgatttccttgaacatttgagcaaagacgataacaaagcatggtatggcgcggaagaaaccgagagagctgcaaaattagatgcaatagaaacactacttattacagatagtgtactaaaaaggaacgacgtgaaaaaacgtgaaaaatacctagacctaatagagaatagtggaaacaacaatggcaaaatattcgtactcagtacttcaaaaatcacagtgagcaacttgacaaaccaacaataaagcagccgctaccaaacagacaagattcagtatgtaaggtaaatacctttttgcacagttaaactacccaaacttattaaagcttgataaattactgaaattccacctttcagttagattcaggcctcatatagattagatatagggtacgtaacattctgtcaaccaagttgttggaatgaaagtctaaaatgtcatctattcggtagcactcatgttactagtatactgtcacatgcggtgtaacgtggggacataaaacagacatcaaatataatggaagctgaaatgcaaagatcgataatgtaataggaatgaaacatataaaacgaaaggagaagtaatggtaatattagtatgtagaaataccgattcaattttggggattcttatattctcgagagaatttctagtataatctgtatacataatattataggctttaccaacaatggaatttcgacaattatcatattattcaccaattaatcacaagttggtaatgagtttgataacaagttactttcttaacaacgttagtatcgtcaaaacactcggttttactcgagcttgtagcacaataataccgtgtagagttctgtattgttcttcttagtgcttgtatatgctcatcccgaccttccattcggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagatgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctatgccaagagaaaacttcttgaagtgtttctctaagcatatcccaaacaacgttgctaacccaaagttggtctatactcaacacgatcaattgtatatgtctattttgaactctactattcaaaatttgagattcatttctgataccaccccaaaaccattggtcattgtcactccttccaacaactcccatatccaagctactattttgtgttctaagaaggtcggtttgcaaatcagaaccagatctggtggtcacgatgccgagggtatgtcctacatttctcaagttccttttgtcgttgttgatttgagaaacatgcactccatcaaaattgatgttcactcccaaaccgcctgggtcgaagctggtgccactttgggtgaagtctactactggattaatgaaaagaatgaaaatttgtccttcccaggtggttactgtccaaccgttggtgttggtggtcacttctccggtggtggttacggtgctttgatgagaaactacggtttagctgctgataacattatcgacgcccacttggttaatgttgatggtaaagtcttggacagaaaatctatgggtgaagatttgttttgggccattagaggtggtggtggtgagaacttcggtatcattgctgcctggaagatcaaattggttgctgttccatctaagtccactattttttccgttaagaagaacatggaaatccatggtttagttaaattatttaacaagtggcaaaacattgcctacaagtacgataaagatttggttttgatgacccatttcattactaagaatattactgacaatcacggtaagaacaagactaccgttcacggttacttctcttctattttccacggtggtgtcgactccttagttgatttgatgaacaaatcctttccagagttgggtattaagaagactgattgtaaggaattctcttggattgacaccaccatcttctactctggtgtcgtcaactttaacactgccaactttaagaaggaaattttattggatagatccgctggtaagaagaccgctttttccatcaaattggattacgtcaagaagccaatccctgagactgccatggtcaagatcttggaaaagttgtatgaagaagacgttggtgctggtatgtacgttttatatccatacggtggtattatggaagagatttccgaatccgctattccattcccacatagagctggtatcatgtacgaattgtggtacactgcttcttgggaaaaacaagaagataatgaaaagcatattaactgggttagatccgtttacaacttcaccactccatatgtttctcaaaacccaagattggcttacttgaactatagagatttggatttgggtaagactaaccacgcctctccaaacaattacactcaagctagaatctggggtgaaaaatacttcggtaagaacttcaacagattagtcaaagtcaagaccaaagttgacccaaacaacttcttcagaaacgagcaatccattcctccattgcctccacaccatcacggccatcatcaccatcaccattagctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgctttgcgaaaccctatgctctgttgttcggatttgaaattttaaaactacattaatgtgttagtttttctttctttctttctttgtcttgacgtgatttggacttctgtcttgcattcgcgtccattcatctgacccaatattccttttggttttgttatccttataaaaagaaaggaagcttcttagagggaaaaaaatgatgaagagtaatgccaaaatataaataaataaataaatatgaaaatcattttctatttttaatagaataagaagagcatcttaagattacaatttcaagaaatagtttacacagtatatccaataactccaataaactactttcctatacaaatttctatggtgggattaatagtaaaacttctgtacttctctaattcaccaagaaattaaggtaaacatctggtaagcactatccagctttttgctattacacatatggcttttctgcaatcatttcttcccattttgtctcaagccgttagtcttgaaaccacaggcggagtagagttacttgatgcggtattttacatgccttttttcactgcaaaaaaaatgaaatacatatttacacgatttgcaggacagtttacgatagtgagtatgcagaatagttaacacctttgttttatccttttgtgtcttaattatatgatataaaggcgcctggccaatagcttgcagcgtagctaaactctaaaatttatctaaatcactcatataaaccgaacccttccccttccgcttatagtacagtacctatacatttcataaacatggcatggcgatcagcgccaaacaatatggaaaatccacagaaagctattcattgaaaaaatagtacaaataagtcacatgatgatatttgattttattatatttttaaaaaaagtaaaaaataaaaagtagtttatttttaaaaaataaaatttaaaatattagtgtatttgatttccgaaagttaaaaaagaaatagtaagaaatatatatttcattgaatggatatatgaaacgtttactggtggaagttttgctc

SEQ ID NO: 88 - protein, 3147 aa, Artificial Sequence
  NPGA
MNKNSKIQSPNSSDVAVIGVGFRFPGNSNDPESLWNNLLDGFDAITQVPKERWATSFREMGLIKNKFGGFLKDSEWKNFDPLFFGIGPKEAPFIDPQQRLLLSIVWESLEDAYIRPDELRGSNTGVFIGVSNNDYTKLGFQDNYSISPYTMTGSNSSLNSNRISYCFDFRGPSITVDTACSSSLVSVNLGVQSIQMGECKIAICGGVNALFDPSTSVAFSKLGVLSENGRCNSFSDQASGYVRSEGAGVVVLKSLEQAKLDGDRIYGVIKGVSSNEDGASNGDKNSLTTPSCEAQSINISKAMEKASLSPSDIYYIEAHGTGTPVGDPIEVKALSKIFSNSNNNQLNNFSTDGNDNDDDDDDNTSPEPLLIGSFKSNIGHLESAAGIASLIKCCLMLKNRMLVPSINCSNLNPSIPFDQYNISVIREIRQFPTDKLVNIGINSFGFGGSNCHLIIQEYNNNFKNNSTICNNNNNNNNNIDYLIPISSKTKKSLDKYLILIKTNSNYHKDISFDDFVKFQIKSKQYNLSNRMTTIANDWNSFIKGSNEFHNLIESKDGEGGSSSSNRGIDSANQINTTTTSTINDIEPLLVFVFCGQGPQWNGMIKTLYNSENVFKNTVDHVDSILYKYFGYSILNVLSKIDDNDDSINHPIVAQPSLFLLQIGLVELFKYWGIYPSISVGHSFGEVSSYYLSGIISLETACKIVYVRSSNQNKTMGSGKMLVVSMGFKQWNDQFSAEWSDIEIACYNAPDSIVVTGNEERLKELSIKLSDESNQIFNTFLRSPCSFHSSHQEVIKGSMFEELSNLQSTGETEIPLFSTVTGRQVLSGHVTAQHIYDNVREPVLFQKTIESITSYIKSHYPSNQKVIYVEIAPHPTLFSLIKKSIPSSNKNSSSVLCPLNRKENSNNSYKKFVSQLYFNGVNVDFNFQLNSICDNVNNDHHLNNVKQNSFKETTNSLPRYQWEQDEYWSEPLISRKNRLEGPTTSLLGHRIIYSFPVFQSVLDLQSDNYKYLLDHLVNGKPVFPGAGYLDIIIEFFDYQKQQLNSSDSSNSYIINVDKIQFLNPIHLTENKLQTLQSSFEPIVTKKSAFSVNFFIKDTVEDQSKVKSMSDETWTNTCKATISLEQQQPSPSSTLTLSKKQDLQILRNRCDISKLDKFELYDKISKNLGLQYNSLFQVVDTIETGKDCSFATLSLPEDTLFTTILNPCLLDNCFHGLLTLINEKGSFVVESISSVSIYLENIGSFNQTSVGNVQFYLYTTISKATSFSSEGTCKLFTKDGSLILSIGKFIIKSTNPKSTKTNETIESPLDETFSIEWQSKDSPIPTPQQIQQQSPLNSNPSFIRSTILKDIQFEQYCSSIIHKELINHEKYKNQQSFDINSLENHLNDDQLMESLSISKEYLRFFTRIISIIKQYPKILNEKELKELKEIIELKYPSEVQLLEFEVIEKVSMIIPKLLFENDKQSSMTLFQDNLLTRFYSNSNSTRFYLERVSEMVLESIRPIVREKRVFRILEIGAGTGSLSNVVLTKLNTYLSTLNSNGGSGYNIIIEYTFTDISANFIIGEIQETMCNLYPNVTFKFSVLDLEKEIINSSDFLMGDYDIVLMAYVIHAVSNIKFSIEQLYKLLSPRGWLLCIEPKSNVVFSDLVFGCFNQWWNYYDDIRTTHCSLSESQWNQLLLNQSLNNESSSSSNCYGGFSNVSFIGGEKDVDSHSFILHCQKESISQMKLATTINNGLSSGSIVIVLNSQQLTNMKSYPKVIEYIQEATSLCKTIEIIDSKDVLNSTNSVLEKIQKSLLVFCLLGYDLLENNYQEQSFEYVKLLNLISTTASSSNDKKPPKVLLITKQSERISRSFYSRSLIGISRTSMNEYPNLSITSIDLDTNDYSLQSLLKPIFSNSKFSDNEFIFKKGLMFVSRIFKNKQLLESSNAFETDSSNLYCKASSDLSYKYAIKQSMLTENQIEIKVECVGINFKDNLFYKGLLPQEIFRMGDIYNPPYGLECSGVITRIGSNVTEYSVGQNVFGFARHSLGSHVVTNKDLVILKPDTISFSEAASIPVVYCTAWYSLFNIGQLSNEESILIHSATGGVGLASLNLLKMKNQQQQPLTNVYATVGSNEKKKFLIDNFNNLFKEDGENIFSTRDKEYSNQLESKIDVILNTLSGEFVESNFKSLRSFGRLIDLSATHVYANQQIGLGNFKFDHLYSAVDLERLIDEKPKLLQSILQRITNSIVNGSLEKIPITIFPSTETKDAIELLSKRSHIGKVVVDCTDISKCNPVGDVITNFSMRLPKPNYQLNLNSTLLITGQSGLSIPLLNWLLSKSGGNVKNVVIISKSTMKWKLQTMISHFVSGFGIHFNYVQVDISNYDALSEAIKQLPSDLPPITSVFHLAAIYNDVPMDQVTMSTVESVHNPKVLGAVNLHRISVSFGWKLNHFVLFSSITAITGYPDQSIYNSANSILDALSNFRRFMGLPSFSINLGPMKDEGKVSTNKSIKKLFKSRGLPSLSLNKLFGLLEVVINNPSNHVIPSQLICSPIDFKTYIESFSTMRPKLLHLQPTISKQQSSIINDSTKASSNISLQDKITSKVSDLLSIPISKINFDHPLKHYGLDSLLTVQFKSWIDKEFEKNLFTHIQLATISINSFLEKVNGLSTNNNNNNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 89 - protein, 3147 aa, Artificial Sequence
  DiPKS[G1516R]
MNKNSKIQSPNSSDVAVIGVGFRFPGNSNDPESLWNNLLDGFDAITQVPKERWATSFREMGLIKNKFGGFLKDSEWKNFDPLFFGIGPKEAPFIDPQQRLLLSIVWESLEDAYIRPDELRGSNTGVFIGVSNNDYTKLGFQDNYSISPYTMTGSNSSLNSNRISYCFDFRGPSITVDTACSSSLVSVNLGVQSIQMGECKIAICGGVNALFDPSTSVAFSKLGVLSENGRCNSFSDQASGYVRSEGAGVVVLKSLEQAKLDGDRIYGVIKGVSSNEDGASNGDKNSLTTPSCEAQSINISKAMEKASLSPSDIYYIEAHGTGTPVGDPIEVKALSKIFSNSNNNQLNNFSTDGNDNDDDDDDNTSPEPLLIGSFKSNIGHLESAAGIASLIKCCLMLKNRMLVPSINCSNLNPSIPFDQYNISVIREIRQFPTDKLVNIGINSFGFGGSNCHLIIQEYNNNFKNNSTICNNNNNNNNNIDYLIPISSKTKKSLDKYLILIKTNSNYHKDISFDDFVKFQIKSKQYNLSNRMTTIANDWNSFIKGSNEFHNLIESKDGEGGSSSSNRGIDSANQINTTTTSTINDIEPLLVFVFCGQGPQWNGMIKTLYNSENVFKNTVDHVDSILYKYFGYSILNVLSKIDDNDDSINHPIVAQPSLFLLQIGLVELFKYWGIYPSISVGHSFGEVSSYYLSGIISLETACKIVYVRSSNQNKTMGSGKMLVVSMGFKQWNDQFSAEWSDIEIACYNAPDSIVVTGNEERLKELSIKLSDESNQIFNTFLRSPCSFHSSHQEVIKGSMFEELSNLQSTGETEIPLFSTVTGRQVLSGHVTAQHIYDNVREPVLFQKTIESITSYIKSHYPSNQKVIYVEIAPHPTLFSLIKKSIPSSNKNSSSVLCPLNRKENSNNSYKKFVSQLYFNGVNVDFNFQLNSICDNVNNDHHLNNVKQNSFKETTNSLPRYQWEQDEYWSEPLISRKNRLEGPTTSLLGHRIIYSFPVFQSVLDLQSDNYKYLLDHLVNGKPVFPGAGYLDIIIEFFDYQKQQLNSSDSSNSYIINVDKIQFLNPIHLTENKLQTLQSSFEPIVTKKSAFSVNFFIKDTVEDQSKVKSMSDETWTNTCKATISLEQQQPSPSSTLTLSKKQDLQILRNRCDISKLDKFELYDKISKNLGLQYNSLFQVVDTIETGKDCSFATLSLPEDTLFTTILNPCLLDNCFHGLLTLINEKGSFVVESISSVSIYLENIGSFNQTSVGNVQFYLYTTISKATSFSSEGTCKLFTKDGSLILSIGKFIIKSTNPKSTKTNETIESPLDETFSIEWQSKDSPIPTPQQIQQQSPLNSNPSFIRSTILKDIQFEQYCSSIIHKELINHEKYKNQQSFDINSLENHLNDDQLMESLSISKEYLRFFTRIISIIKQYPKILNEKELKELKEIIELKYPSEVQLLEFEVIEKVSMIIPKLLFENDKQSSMTLFQDNLLTRFYSNSNSTRFYLERVSEMVLESIRPIVREKRVFRILEIGARTGSLSNVVLTKLNTYLSTLNSNGGSGYNIIIEYTFTDISANFIIGEIQETMCNLYPNVTFKFSVLDLEKEIINSSDFLMGDYDIVLMAYVIHAVSNIKFSIEQLYKLLSPRGWLLCIEPKSNVVFSDLVFGCFNQWWNYYDDIRTTHCSLSESQWNQLLLNQSLNNESSSSSNCYGGFSNVSFIGGEKDVDSHSFILHCQKESISQMKLATTINNGLSSGSIVIVLNSQQLTNMKSYPKVIEYIQEATSLCKTIEIIDSKDVLNSTNSVLEKIQKSLLVFCLLGYDLLENNYQEQSFEYVKLLNLISTTASSSNDKKPPKVLLITKQSERISRSFYSRSLIGISRTSMNEYPNLSITSIDLDTNDYSLQSLLKPIFSNSKFSDNEFIFKKGLMFVSRIFKNKQLLESSNAFETDSSNLYCKASSDLSYKYAIKQSMLTENQIEIKVECVGINFKDNLFYKGLLPQEIFRMGDIYNPPYGLECSGVITRIGSNVTEYSVGQNVFGFARHSLGSHVVTNKDLVILKPDTISFSEAASIPVVYCTAWYSLFNIGQLSNEESILIHSATGGVGLASLNLLKMKNQQQQPLTNVYATVGSNEKKKFLIDNFNNLFKEDGENIFSTRDKEYSNQLESKIDVILNTLSGEFVESNFKSLRSFGRLIDLSATHVYANQQIGLGNFKFDHLYSAVDLERLIDEKPKLLQSILQRITNSIVNGSLEKIPITIFPSTETKDAIELLSKRSHIGKVVVDCTDISKCNPVGDVITNFSMRLPKPNYQLNLNSTLLITGQSGLSIPLLNWLLSKSGGNVKNVVIISKSTMKWKLQTMISHFVSGFGIHFNYVQVDISNYDALSEAIKQLPSDLPPITSVFHLAAIYNDVPMDQVTMSTVESVHNPKVLGAVNLHRISVSFGWKLNHFVLFSSITAITGYPDQSIYNSANSILDALSNFRRFMGLPSFSINLGPMKDEGKVSTNKSIKKLFKSRGLPSLSLNKLFGLLEVVINNPSNHVIPSQLICSPIDFKTYIESFSTMRPKLLHLQPTISKQQSSIINDSTKASSNISLQDKITSKVSDLLSIPISKINFDHPLKHYGLDSLLTVQFKSWIDKEFEKNLFTHIQLATISINSFLEKVNGLSTNNNNNNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 90 - protein, 102 aa, Artificial Sequence
  OAC Protein
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRKG

SEQ ID NO: 91 - protein, 323 aa, Artificial Sequence
  PT254
MSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 92 - protein, 610 aa, Artificial Sequence
  OstI-pro-alpha-f(I)-OXC53 Protein
MRQVWFSWIVGLFLCFFNVSSAAPVNTTTEDETAQIPAEAVIGYSDLEGDFDVAVLPFSNSTNNGLLFINTTIASIAAKEEGVSLEKREAEAMPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHHG

(19) United States
(12) Patent Application Publication
(10) Pub. No.: US 2023/0257787 A1
(43) Pub. Date: Aug. 17, 2023

(54) METHODS AND CELLS WITH MODIFYING ENZYMES FOR PRODUCING SUBSTITUTED CANNABINOIDS AND PRECURSORS

(71) Applicant: Hyasynth Biologicals Inc., Montréal (CA)

(72) Inventors: Alexander CAMPBELL, Montréal (CA); Sylvester PALYS, Montréal (CA)

(21) Appl. No.: 18/006,171

(22) PCT Filed: Jun. 29, 2021
(86) PCT No.: PCT/CA2021/050894

§ 371 Date: Jan. 20, 2023

Related U.S. Application Data
(60) Provisional application No. 63/056,126, filed on Jul. 24, 2020.

(51) Int. Cl. C12P 17/06; C12P 7/42; C07C 65/05

(52) U.S. Cl. CPC C12P 17/06; C12P 7/42; C07C 65/05

(57) ABSTRACT

The present disclosure relates generally to methods and cells for the production of substituted phytocannabinoids or substituted phytocannabinoid precursors in host cells that produce the phytocannabinoid or the phytocannabinoid precursor. Methods are described which comprise transforming host cells with a sequence encoding an enzyme for derivatizing the phytocannabinoid or precursor with a substituent, such as O-methyl, glycosyl, or halogen. The transformed cells are cultured to produce substituted phytocannabinoids or substituted phytocannabinoid precursors.

### CROSS REFERENCE TO RELATED APPLICATIONS

This application claims the benefit of and priority to U.S. Provisional Patent Application No. 63/056,126, filed Jul. 24, 2020, and entitled METHODS AND CELLS WITH MODIFYING ENZYMES FOR PRODUCING SUBSTITUTED CANNABINOIDS AND PRECURSORS, which is incorporated by reference herein in its entirety.

### FIELD

The present disclosure relates generally to the production of phytocannabinoids in host cells using heterologous enzymes. Methods and cell lines for the production of phytocannabinoids, as well as the products so formed, are described.

### BACKGROUND

Phytocannabinoids are naturally produced in Cannabis sativa, other plants, and some fungi. Over 105 phytocannabinoids are known to be biosynthesized in C. sativa, or result from thermal or other decomposition from phytocannabinoids biosynthesized in C. sativa. While the C. sativa plant is also a valuable source of grain, fiber, and other material, growing C. sativa for phytocannabinoid production, particularly indoors, is costly in terms of energy and labour. Subsequent extraction, purification, and fractionation of phytocannabinoids from the C. sativa plant is also labour and energy intensive.

Phytocannabinoids are pharmacologically active molecules that contribute to the medical and psychotropic effects of C. sativa. Biosynthesis in the C. sativa plant scales similarly to other agricultural projects. As with other agricultural projects, large scale production of phytocannabinoids by growing C. sativa requires a variety of inputs (e.g. nutrients, light, pest control, CO₂, etc.). The inputs required for cultivating C. sativa must be provided. In addition, cultivation of C. sativa, where allowed, is currently subject to heavy regulation, taxes, and rigorous quality control where products prepared from the plant are for commercial use, further increasing costs. As a result, it may be economical to produce the phytocannabinoids in a robust and scalable, fermentable organism. Saccharomyces cerevisiae has been used to produce industrial scales of similar molecules.

The time, energy, and labour involved in growing C. sativa for phytocannabinoid production provides a motivation to produce transgenic cell lines for production of phytocannabinoids in yeast. One example of such efforts is provided in the PCT patent application of Mookerjee et al. WO2018/148848, which is hereby incorporated by reference.

An advantage of yeast based biosynthesis is that strains can be easily modified to synthesize cannabinoids not typically produced in high quantities by C. sativa. Cannabinoids other than CBGa, THCa and CBDa are often collectively termed the “minor cannabinoids” and many members of this class have significant applications in medicine and related fields. For example THCV has potential use in the treatment of diabetes, Parkinson's disease and epilepsy (Wargent et al., 2013; Garcia et al., 2011; U.S. Pat. No. 9,066,920 all of which are hereby incorporated by reference).

It is desirable to find alternate methods for the production of phytocannabinoids, and/or for the production of compounds useful in phytocannabinoid synthesis as intermediate or precursor compounds, such as aromatic polyketides.

### SUMMARY

Methods, cells, and other aspects are described for producing phytocannabinoids. In particular, the production of O-methylated cannabinoids and cannabinoid precursors using an enzymes OMT1-OMT30 is described; production of glycosylated cannabinoids and cannabinoid precursors using an enzyme selected from GLY1-GLY11 is described, and production of halogenated cannabinoids and cannabinoid precursors using an enzyme selected from HAL1-HAL20 is described.

There is provided herein a method of producing a substituted phytocannabinoid or a substituted phytocannabinoid precursor in a host cell that produces the phytocannabinoid or the phytocannabinoid precursor, said method comprising: transforming said host cell with a sequence encoding an enzyme for derivatizing the phytocannabinoid or the phytocannabinoid precursor with the substituent, and cuturing said transformed host cell to produce said substituted phytocannabinoid or said substituted phytocannabinoid precursor.

Further, there is provided a method of producing a substituted phytocannabinoid or substituted phytocannabinoid precursor, comprising: providing a host cell capable of producing the phytocannabinoid or phytocannabinoid precursor; introducing into the host cell a polynucleotide encoding an enzyme for derivatizing said phytocannabinoid or phytocannabinoid precursor; and culturing the host cell under conditions sufficient for production of the substituted phytocannabinoid or substituted phytocannabinoid precursor.

An expression vector is described herein comprising a nucleotide molecule comprising a polynucleotide sequence encoding an enzyme for derivatizing a phytocannabinoid or a phytocannabinoid precursor with the substituent, wherein said nucleotide sequence encodes an enzyme having an amino acid sequence according to any one of SEQ ID NO:1-SEQ ID NO:62 or encoding an enzyme having at least 70% identity thereto.

Further, there is provided a host cell transformed with the expression vector.

Unique products formed according to the described method, such as chlorinated olivetolic acid, are also provided herein.

According to one aspect, there is described herein a method of producing a substituted phytocannabinoid or a substituted phytocannabinoid precursor in a host cell that produces the phytocannabinoid or the phytocannabinoid precursor. The method comprises: transforming said host cell with a sequence encoding an enzyme for derivatizing the phytocannabinoid or the phytocannabinoid precursor with the substituent, and culturing said transformed host cell to produce said substituted phytocannabinoid or said substituted phytocannabinoid precursor, wherein: the derivatizing comprises:

(i) O-methylation, wherein the substituent is O-methyl, and the enzyme for derivatizing comprises an O-methylation enzyme selected from the group consisting of an OMT1-OMT30 protein comprising an amino acid sequence of at least 95% identity to the sequence as set forth in any one of SEQ ID NO:1-SEQ ID NO:30;

(ii) glycosylation, wherein the substituent is glycosyl, and the enzyme for derivatizing comprises a glycosylation enzyme selected from the group consisting of a GLY1-GLY11 protein comprising an amino acid sequence of at least 95% identity to the sequence as set forth in any one of SEQ ID NO:31-SEQ ID NO:41; or

(iii) halogenation, wherein the substituent is halogen, and the enzyme for derivatizing comprises a halogenation enzyme selected from the group consisting of a HAL1-HAL20 protein comprising an amino acid sequence of at least 95% identity to the sequence as set forth in any one of SEQ ID NO:42-SEQ ID NO:62.

Other aspects and features of the present disclosure will become apparent to those ordinarily skilled in the art upon review of the following description of specific embodiments in conjunction with the accompanying figures.

### BRIEF DESCRIPTION OF THE FIGURE(S)

Embodiments of the present disclosure will now be described, by way of example only, with reference to the Figure(s).

FIG. 1 depicts a generalized scheme in which glycosylases, halogenases and O-methyltransferases can be used to derivatize cannabinoids or cannabinoid precursors in a THCa producing yeast strain.

### DETAILED DESCRIPTION

Modifying enzymes can be inserted in organisms to produce desirable modified phytocannabinoids or precursors. By incorporating such enzymes, a glycosylation, halogenation and/or O-methylation reaction can occur in a cannabinoid producing yeast strain. Glycosylation, O-methylation and halogenation are three exemplary types of chemical modifications that can be attained by enzymatic derivatization of naturally occurring phytocannabinoids leading to useful products. Methods of producing O-methylated cannabinoids and precursors can be employed using an enzyme selected from OMT1-OMT30, production of glycosylated cannabinoids and precursors can be done using an enzyme selected from GLY1-GLY11. Further, halogenated cannabinoids and precursors may be prepared using an enzyme selected from HAL1-HAL20, as described herein. In the case of glycosylations, the reaction can occur at a free alcohol group.

A method of producing a substituted phytocannabinoid or a substituted phytocannabinoid precursor in a host cell that produces the phytocannabinoid or the phytocannabinoid precursor is described. The method comprises: transforming the host cell with a sequence encoding an enzyme for derivatizing the phytocannabinoid or the phytocannabinoid precursor with the substituent, and culturing the transformed host cell to produce a substituted phytocannabinoid or a substituted phytocannabinoid precursor.

The derivatizing may comprise O-methylation, glycosylation, or halogenation. The enzyme encoded may comprise or consist of (a) an O-methylation enzyme selected from the group consisting of an OMT1-OMT30 protein comprising an amino acid sequence as set forth in any one of SEQ ID NO:1-SEQ ID NO:30; a glycosylation enzyme selected from the group consisting of a GLY1-GLY11 protein comprising an amino acid sequence as set forth in any one of SEQ ID NO:31-SEQ ID NO:41; or a halogenation enzyme selected from the group consisting of a HAL1-HAL20 protein comprising an amino acid sequence as set forth in any one of SEQ ID NO:42-SEQ ID NO:62; (b) an enzyme comprising an amino acid sequence with at least 70% identity with a protein set forth in (a); (c) an enzyme comprising an amino acid sequence that differs from a protein set forth in (a) by one or more amino acid residues that is substituted, deleted and/or inserted; or (d) an enzyme that is a derivative of (a), (b), or (c).

The method may involve the O-methylation enzyme comprising or consisting of an amino acid sequence with at least 75%, 80%, 85%, 90%, 95%, 96%, 97%, 98%, or 99% identity with the amino acid sequence set forth in (a), for example with at least 85% or at least 95% identity thereto.

The sequence encoding the enzyme may comprise or consist of a nucleotide sequence encoding any one of SEQ ID NO:1-SEQ ID NO:62 or encoding an enzyme having at least 70% identity thereto. For example, the nucleotide sequence may encode an enzyme having at least 85% or at least 95% sequence identity to any one of SEQ ID NO:1-SEQ ID NO:62.

The phytocannabinoid employed in the method may be an acid, and may optionally be selected from the group consisting of cannabigorcinic acid (CBGOa) tetrahydrocannabinolic acid (THCa), cannabidiolic acid (CBDa), cannabichromenic acid (CBCa), cannabigerolic acid (CBGa), cannabigerovarinic acid (CBGVa), tetrahydrocannabivarin acid (THCVa), tetrahydrocannabiorsellenic acid (THCOa), and cannabidiorsellenic acid. Preferably, the phytocannabinoid or phytocannabinoid precursor may comprise cannabigerolic acid (CBGa), cannabidiolic acid (CBDA), tetrahydrocannabinolic acid (THCA), or olivetolic acid.

The substituent with which the phytocannabinoid or precursor is derivatized may be, for example O-methyl, glycosyl or halogen. The substituted phytocannabinoid or substituted phytocannabinoid precursor may be O-methylated THCa, glycosylated CBGa, or chlorinated olivetolic acid. For example, the protein may comprise an O-methylation enzyme with an amino acid sequence with least 85% or at least 95% sequence identity with SEQ ID NO:1-SEQ ID NO:30. Further, the protein may comprise a glycosylation enzyme with an amino acid sequence with least 85% or at least 95% sequence identity with SEQ ID NO:31-SEQ ID NO:41. Further, the protein may comprise a halogenation enzyme with an amino acid sequence with least 85% or at least 95% sequence identity with SEQ ID NO:42-SEQ ID NO:62.

In the method described, exemplary embodiments include when the phytocannabinoid is THCa and the substituent is O-methyl; when the phytocannabinoid is CBGa, and the substituent is glycosyl; and when the phytocannabinoid precursor is olivetolic acid and the substituent is a halogen, such as chlorine.

In the method described, the host cell may additionally comprises a nucleic acid encoding a protein having an amino acid sequence according to any one of SEQ ID NO:88-SEQ ID NO:92.

The host cell may be a bacterial cell, a fungal cell, a protist cell, or a plant cell. For example, the bacterial cell may be from Escherichia coli, Streptomyces coelicolor, Bacillus subtilis, Mycoplasma genitalium, Synechocytis, Zymomonas mobilis, Corynebacterium glutamicum, Synechococcus sp., Salmonella typhi, Shigella flexneri, Shigella sonnei, Shigella disenteriae, Pseudomonas putida, Pseudomonas aeruginosa, Pseudomonas mevalonii, Rhodobacter sphaeroides, Rhodobacter capsulatus, Rhodospirillum rubrum, or Rhodococcus sp. Exemplary fungal cells include Saccharomyces cerevisiae, Ogataea polymorpha, Komagataella phaffii, Kluyveromyces lactis, Neurospora crassa, Aspergillus niger, Aspergillus nidulans, Schizosaccharomyces pombe, Yarrowia lipolytica, Myceliophthora thermophila, Aspergillus oryzae, Trichoderma reesei, Chrysosporium lucknowense, Fusarium sp., Fusarium gramineum, Fusarium venenatum, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stipitis, Pichia methanolica, or Hansenula polymorpha. Possible protist host cells include Chlamydomonas reinhardtii, Dictyostelium discoideum, Chlorella sp., Haematococcus pluvialis, Arthrospira platensis, Dunaliella sp., or Nannochloropsis oceanica. Exemplary plant cells include Cannabis sativa, Arabidopsis thaliana, Theobroma cacao, maize, banana, peanut, field peas, sunflower, Nicotiana sp., tomato, canola, wheat, barley, oats, potato, soybeans, cotton, sorghum, lupin, or rice.

For example, the host cell may be S. cerevisiae, E. coli, Yarrowia lipolytica, or Komagataella phaffii.

A method of producing a substituted phytocannabinoid or substituted phytocannabinoid precursor is described herein. The method includes the steps of providing a host cell capable of producing the phytocannabinoid or phytocannabinoid precursor; introducing into the host cell a polynucleotide encoding an enzyme for derivatizing said phytocannabinoid or phytocannabinoid precursor; and culturing the host cell under conditions sufficient for production of the substituted phytocannabinoid or substituted phytocannabinoid precursor.

The host cell may additionally comprise one or more genetic modifications, such as: (a) a nucleic acid as set forth in any one of SEQ ID NO:73 to SEQ ID NO:87; (b) a nucleic acid having at least 70% identity with the nucleotide sequence of (a); (c) a nucleic acid that hybridizes with the complementary strand of the nucleic acid of (a); (d) a nucleic acid encoding a polypeptide with the same enzyme activity as the polypeptide encoded by any one of the nucleic acid sequences of (a); (e) a nucleotide sequence that differs from (a) by one or more nucleotides that are substituted, deleted, and/or inserted; or (f) a derivative of (a), (b), (c), (d), or (e).

For example, the method may involve at least one genetic modification comprises a nucleic acid having at least 75%, 80%, 85%, 90%, 95%, 96%, 97%, 98%, or 99% identity with the nucleic acid as set forth in (a), such as for example at least 85% or at least 95% sequence identity thereto.

The host cell may additionally comprise a nucleic acid encoding a protein having an amino acid sequence with at least 85% or at least 95% identity with a sequence according to any one of SEQ ID NO:88-SEQ ID NO:92. The host cell may further comprise a plasmid with a nucleotide sequence with at least 85% or at least 95% identity with a sequence according to any one of SEQ ID NO:64-SEQ ID NO:72.

An expression vector is provided, comprising a nucleotide molecule comprising a polynucleotide sequence encoding an enzyme for derivatizing a phytocannabinoid or a phytocannabinoid precursor with the substituent, wherein said nucleotide sequence encodes an enzyme having an amino acid sequence according to any one of SEQ ID NO:1-SEQ ID NO:62 or encoding an enzyme having at least 70% identity thereto, for example at least 75%, 80%, 85%, 90%, 95%, 96%, 97%, 98%, or 99% identity thereto. Such an expression vector may include nucleotide sequence encodeing enzyme having at least 85% or at least 95% sequence identity with any one of SEQ ID NO:1-SEQ ID NO:62. Further, the expression vector may comprise a nucleotide sequence according to any one of SEQ ID NO:64-SEQ ID NO:72.

A host cell transformed with the expression vector is also described. The host cell may comprising one or more of: (a) a nucleic acid as set forth in any one of SEQ ID NO: 73 to SEQ ID NO:87; (b) a nucleic acid having at least 70% identity, for example at least 85% or at least 95%, with the nucleotide sequence of (a); (c) a nucleic acid that hybridizes with the complementary strand of the nucleic acid of (a); (d) a nucleic acid encoding a protein with the same enzyme activity as the protein encoded by any one of the nucleic acid sequences of (a); (e) a nucleic acid that differs from (a) by one or more nucleotides that are substituted, deleted, and/or inserted; or (f) a derivative of (a), (b), (c), (d), or (e). Further, the host cell may comprise a nucleotide sequence encoding a protein with an amino acid sequence according to any one of SEQ ID NO:88-SEQ ID NO:92.

The host cell may be a bacterial cell, a fungal cell, a protist cell, or a plant cell. Exemplary host cells may be S. cerevisiae, E. coli, Yarrowia lipolytica, or Komagataella phaffii.

A halogenated olivetolic acid may be produced by the method described, or more particularly, a chlorinated olivetolic acid.

One advantage of yeast based biosynthesis is that strains can be easily modified to synthesize cannabinoids not typically produced in high quantities by C. sativa. Cannabinoids outside of CBGa, THCa and CBDa are often collectively termed the “minor cannabinoids”.

Minor cannabinoids can be synthesized using a number of different biosynthetic routes, one of which is by positional substitution or derivatization of an existing cannabinoid by a modifying enzyme.

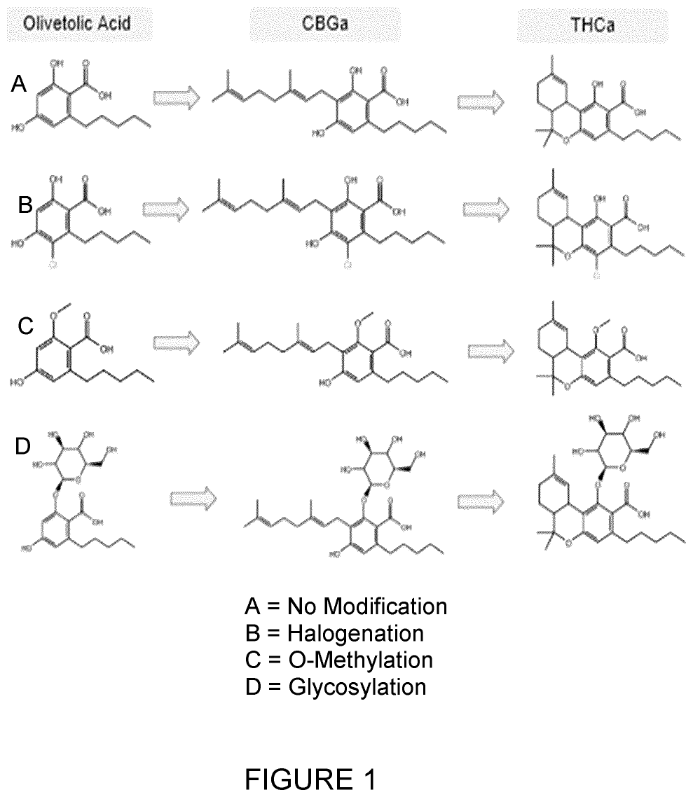

FIG. 1 illustrates biosynthetic routes without modification (Panel A), synthetic routes involving halogenases (Panel B), O-methyltransferases (Panel C), and glycosylases (Panel D). These biosynthetic routes can be used to derivatize cannabinoids or cannabinoid precursors in a THCa producing yeast strain. The modifications can potentially occur at any step in the pathway. As illustrated, glycosylation, halogenation and O-methylation reactions can occur in a THCa producing yeast strain such as in strain HB1254, as described Applicant's co-pending PCT Patent Appln No. CA2020/050687 entitled METHODS AND CELLS FOR PRODUCTION OF PHYTOCANNABINOIDS AND PHYTOCANNABINOID PRECURSORS filed May 21, 2020 and hereby incorporated by reference. In the case of glycosylations, the reaction can occur at either free alcohol group (for CBGa or olivetolic acid).

O-methylated cannabinoids occur naturally in C. sativa with quantities varying from strain to strain (Caprioglio et al., 2019; Yamauchi et al., 1968). There has been little research into the biochemical properties of O-methylated cannabinoids, though O-dimethyl CBDA is noted as a potent and selective 15-LOX inhibitor (Takeda et al., 2009) and may have applications in obesity, atherosclerosis and cancer. Glycosylated and halogenated cannabinoids do not occur naturally in C. sativa, though glycosylated cannabinoids can be produced in C. sativa through the heterologous expression of glycosyltransferases (Hardman et al., 2017).

Glycosylated cannabinoids have improved water solubility and the expression of glycosyltransferases is noted to greatly improve cannabinoid yields in planta (US2019/0338301 A1 of Sayre et al.) Both glycosylation and halogenation improve cannabinoid solubility and these enzymes may also improve titres in yeast. Halogenated cannabinoids have not been made biosynthetically although halogenated prenylated polyketides with similar structures exist in nature, such as ilicicolinic acid (Okada et al, 2017). Halogenated analogues of CBD have been shown to have sedative and anticonvulsant properties (Usami et al., 1999).

Glycosylation, O-methylation and halogenation are three exemplary types of chemical diversity that can be attained by enzymatic derivatization of naturally occurring phytocannabinoids.

The modifications described herein may also be achieved with differing cannabinoid backbones such as CBDa or CBCa.

Certain terms used herein are described below.

The term “cannabinoid” as used herein refers to a chemical compound that shows direct or indirect activity at a cannabinoid receptor. Non limiting examples of cannabinoids include tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), tetrahydrocannabivarin (THCV), tetrahydrocannabiorsellenol (THCO), cannabidiorsellenol, and cannabigerol monomethyl ether (CBGM). Acids of these are included within the term “cannabinoid”.

The term “phytocannabinoid” as used herein refers to a cannabinoid, including an acid form, that typically occurs in a plant species. Exemplary phytocannabinoids produced according to the invention include cannabigerol (CBG), cannabigerolic acid (CBGa), cannabigerovarin (CBGv), cannabigerovarinic acid (CBGva), cannabigerocin (CBGo), or cannabigerocinic acid (CBGoa).

Cannabinoids and phytocannabinoids may contain or may lack one or more carboxylic acid functional groups. Non limiting examples of such cannabinoids or phytocannabinoids containing carboxylic acid function groups or phytocannabinoids include tetrahydrocannabinolic acid (THCa), cannabidiolic acid (CBDA), and cannabichromenic acid (CBCA).

The term “homologue” includes homologous sequences from the same and other species and orthologous sequences from the same and other species. Different polynucleotides or polypeptides having homology may be referred to as homologues.

The term “homology” may refer to the level of similarity between two or more polynucleotide and/or polypeptide sequences in terms of percent of positional identity (i.e., sequence similarity or identity). Homology also refers to the concept of similar functional properties among different polynucleotide or polypeptides. Thus, the compositions and methods herein may further comprise homologues to the polypeptide and polynucleotide sequences described herein.

The term “orthologous,” as used herein, refers to homologous polypeptide sequences and/or polynucleotide sequences in different species that arose from a common ancestral gene during speciation.

As used herein, a “homologue” may have a significant sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and/or 100%) to the polynucleotide sequences herein.

As used herein “sequence identity” refers to the extent to which two optimally aligned polynucleotide or peptide sequences are invariant throughout a window of alignment of components, e.g., nucleotides or amino acids. “Identity” can be readily calculated by known methods.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

The terms “fatty acid-CoA”, “fatty acyl-CoA”, or “CoA donors” as used herein may refer to compounds useful in polyketide synthesis as primer molecules which react in a condensation reaction with an extender unit (such as malonyl-CoA) to form a polyketide. Examples of fatty acid-CoA molecules (also referred to herein as primer molecules or CoA donors), useful in the synthetic routes described herein include but are not limited to: acetyl-CoA, butyryl-CoA, hexanoyl-CoA . These fatty acid-CoA molecules may be provided to host cells or may be synthesized by the host cells for biosynthesis of polyketides, as described herein.

Two nucleotide sequences can be considered to be substantially “complementary” when the two sequences hybridize to each other under stringent conditions. In some examples, two nucleotide sequences considered to be substantially complementary hybridize to each other under highly stringent conditions.

The terms “stringent hybridization conditions” and “stringent hybridization wash conditions” in the context of nucleic acid hybridization experiments, for example in Southern hybridizations and Northern hybridizations are sequence dependent, and are different under different environmental parameters. In some examples, generally, highly stringent hybridization and wash conditions are selected to be about 5° C. lower than the thermal melting point (Tm) for the specific sequence at a defined ionic strength and pH.

In some examples, polynucleotides include polynucleotides or “variants” having at least about 50%, 55%, 60%, 65%, 70%, 75%, 80%, 85%, 90%, 95%, 96%, 97%, 98%, 99% or 100% sequence identity to any of the reference sequences described herein, typically where the variant maintains at least one biological activity of the reference sequence.

As used herein, the terms “polynucleotide variant” and “variant” and the like refer to polynucleotides displaying substantial sequence identity with a reference polynucleotide sequence or polynucleotides that hybridize with a reference sequence under, for example, stringent conditions. These terms may include polynucleotides in which one or more nucleotides have been added or deleted, or replaced with different nucleotides compared to a reference polynucleotide. It will be understood that that certain alterations inclusive of mutations, additions, deletions and substitutions can be made to a reference polynucleotide whereby the altered polynucleotide retains the biological function or activity of the reference polynucleotide.

In some examples, the polynucleotides described herein may be included within “vectors” and/or “expression cassettes”.

In some embodiments, the nucleotide sequences and/or nucleic acid molecules described herein may be “operably” or “operatively” linked to a variety of promoters for expression in host cells. Thus, in some examples, the invention provides transformed host cells and transformed organisms comprising the transformed host cells, wherein the host cells and organisms are transformed with one or more nucleic acid molecules/nucleotide sequences of the invention. As used herein, “operably linked to,” when referring to a first nucleic acid sequence that is operably linked to a second nucleic acid sequence, means a situation when the first nucleic acid sequence is placed in a functional relationship with the second nucleic acid sequence. For instance, a promoter is operably associated with a coding sequence if the promoter effects the transcription or expression of the coding sequence.

In the context of a polypeptide, “operably linked to,” when referring to a first polypeptide sequence that is operably linked to a second polypeptide sequence, refers to a situation when the first polypeptide sequence is placed in a functional relationship with the second polypeptide sequence.

The term a “promoter,” as used herein, refers to a nucleotide sequence that controls or regulates the transcription of a nucleotide sequence (i.e., a coding sequence) that is operably associated with the promoter. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression.

Promoters can include, for example, constitutive, inducible, temporally regulated, developmentally regulated, chemically regulated, tissue-preferred and tissue-specific promoters for use in the preparation of recombinant nucleic acid molecules, i.e., chimeric genes.

The choice of promoter will vary depending on the temporal and spatial requirements for expression, and also depending on the host cell to be transformed. Thus, for example, where expression in response to a stimulus is desired a promoter inducible by stimuli or chemicals can be used. Where continuous expression at a relatively constant level is desired throughout the cells or tissues of an organism a constitutive promoter can be chosen.

In some examples, vectors may be used.

In some examples, the polynucleotide molecules and nucleotide sequences described herein can be used in connection with vectors.

The term “vector” refers to a composition for transferring, delivering or introducing a nucleic acid or polynucleotide into a host cell. A vector may comprise a polynucleotide molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid, a fosmid, a bacteriophage, or an artificial chromosome. The selection of a vector will depend upon the preferred transformation technique and the target species for transformation.

As used herein, “expression vectors” refers to a nucleic acid molecule comprising a nucleotide sequence of interest, wherein said nucleotide sequence is operatively associated with at least a control sequence (e.g., a promoter). Thus, some examples provide expression vectors designed to express the polynucleotide sequences of described herein.

An expression vector comprising a polynucleotide sequence of interest may be “chimeric”, meaning that at least one of its components is heterologous with respect to at least one of its other components. An expression cassette may also be one that is naturally occurring but has been obtained in a recombinant form useful for heterologous expression. In some examples, however, the expression vector is heterologous with respect to the host. For example, the particular polynucleotide sequence of the expression vector does not occur naturally in the host cell and must have been introduced into the host cell or an ancestor of the host cell by a transformation event.

In some examples, an expression vector may also include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, promoters, enhancers, introns, 5′ and 3′ untranslated regions, translation leader sequences, termination signals, and polyadenylation signal sequences.

An expression vector may also include a nucleotide sequence for a selectable marker, which can be used to select a transformed host cell.

As used herein, “selectable marker” means a nucleotide sequence that when expressed imparts a distinct phenotype to the host cell expressing the marker and thus allows such transformed host cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, a sugar, a carbon source, or the like), or on whether the marker is simply a trait that one can identify through observation or testing, such as by screening. Examples of suitable selectable markers are known in the art and can be used in the expression vectors described herein.

The vector and/or expression vectors and/or polynucleotides may be introduced in to a cell.

The term “introducing,” in the context of a nucleotide sequence of interest (e.g., the nucleic acid molecules/constructs/expression vectors), refers to presenting the nucleotide sequence of interest to cell host in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different transformation vectors. Accordingly, these polynucleotides may be introduced into host cells in a single transformation event, or in separate transformation events.

As used herein, the term “contacting” refers to a process by which, for example, a compound may be delivered to a cell. The compound may be administered in a number of ways, including, but not limited to, direct introduction into a cell (i.e., intracellularly) and/or extracellular introduction into a cavity, interstitial space, or into the circulation of the organism.

The term “transformation” or “transfection” as used herein refers to the introduction of a polynucleotide or heterologous nucleic acid into a cell. Transformation of a cell may be stable or transient.

The term “transient transformation” as used herein in the context of a polynucleotide refers to a polynucleotide introduced into the cell and does not integrate into the genome of the cell.

The terms “stably introducing” or “stably introduced” in the context of a polynucleotide introduced into a cell is intended to represent that the introduced polynucleotide is stably incorporated into the genome of the cell, and thus the cell is stably transformed with the polynucleotide.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transformed in vivo or in vitro with a recombinant vector or a polynucleotide of the invention. A host cell which comprises a recombinant vector of the invention is a recombinant host cell.

In some examples, a host cell may be a bacterial cell, a fungal cell, a protist cell, or a plant cell. Specific examples of host cells are described below.

The host cell can be a bacterial cell, a fungal cell, a protist cell, or a plant cell, such as any of the exemplary cell types noted herein in Table 1. Exemplary host cell types include S. cerevisiae, E. coli, Yarrowia lipolytica, and Komagataella phaffii.

TABLE 1
List of Host Cell Organisms

| Type | Organisms |
|---|---|
| Bacteria | Escherichia coli, Streptomyces coelicolor and other species., Bacillus |
| | subtilis, Mycoplasma genitalium, Synechocytis, Zymomonas mobilis, |
| | Corynebacterium glutamicum, Synechococcus sp., Salmonella typhi, |
| | Shigella flexneri, Shigella sonnei, and Shigella disenteriae, Pseudomonas |
| | putida, Pseudomonas aeruginosa, Pseudomonas mevalonii, Rhodobacter |
| | sphaeroides, Rhodobacter capsulatus, Rhodospirillum rubrum, |
| | Rhodococcus sp. |
| Fungi | Saccharomyces cerevisiae, Ogataea polymorpha, Komagataella phaffii, |
| | Kluyveromyces lactis, Neurospora crassa, Aspergillus niger, Aspergillus |
| | nidulans, Schizosaccharomyces pombe, Yarrowia lipolytica, |
| | Myceliophthora thermophila, Aspergillus oryzae, Trichoderma reesei, |
| | Chrysosporium lucknowense, Fusarium sp., Fusarium gramineum, |
| | Fusarium venenatum, Pichia finlandica, Pichia trehalophila, Pichia |
| | koclamae, Pichia membranaefaciens, Pichia opuntiae, Pichia |
| | thermotolerans, Pichia salictaria, Pichia guercuum, Pichia pijperi, Pichia |
| | stipitis, Pichia methanolica, Hansenula polymorpha. |
| Protists | Chlamydomonas reinhardtii, Dictyostelium discoideum, Chlorella sp., |
| | Haematococcus pluvialis, Arthrospira platensis, Dunaliella sp., |
| | Nannochloropsis oceanica. |
| Plants | Cannabis sativa, Arabidopsis thaliana, Theobroma cacao, maize, banana, |
| | peanut, field peas, sunflower, Nicotiana sp., tomato, canola, wheat, barley, |
| | oats, potato, soybeans, cotton, sorghum, lupin, rice. |

Table 2 outlines the sequences described herein.

TABLE 2
Description of Sequences

| | | DNA/ | Length of | Position |
|---|---|---|---|---|
| SEQ ID NO: | Description | Protein | sequence | of coding sequence |
| SEQ ID NO. 1 | OMT1 | Protein | 348 | All |
| SEQ ID NO. 2 | OMT2 | Protein | 368 | All |
| SEQ ID NO. 3 | OMT3 | Protein | 356 | All |
| SEQ ID NO. 4 | OMT4 | Protein | 370 | All |
| SEQ ID NO. 5 | OMT5 | Protein | 409 | All |
| SEQ ID NO. 6 | OMT6 | Protein | 335 | All |
| SEQ ID NO. 7 | OMT7 | Protein | 177 | All |
| SEQ ID NO. 8 | OMT8 | Protein | 389 | All |
| SEQ ID NO. 9 | OMT9 | Protein | 398 | All |
| SEQ ID NO. 10 | OMT10 | Protein | 366 | All |
| SEQ ID NO. 11 | OMT11 | Protein | 313 | All |
| SEQ ID NO. 12 | OMT12 | Protein | 365 | All |
| SEQ ID NO. 13 | OMT13 | Protein | 374 | All |
| SEQ ID NO. 14 | OMT14 | Protein | 365 | All |
| SEQ ID NO. 15 | OMT15 | Protein | 365 | All |
| SEQ ID NO. 16 | OMT16 | Protein | 365 | All |
| SEQ ID NO. 17 | OMT17 | Protein | 352 | All |
| SEQ ID NO. 18 | OMT18 | Protein | 360 | All |
| SEQ ID NO. 19 | OMT19 | Protein | 439 | All |
| SEQ ID NO. 20 | OMT20 | Protein | 378 | All |
| SEQ ID NO. 21 | OMT21 | Protein | 427 | All |
| SEQ ID NO. 22 | OMT22 | Protein | 378 | All |
| SEQ ID NO. 23 | OMT23 | Protein | 344 | All |
| SEQ ID NO. 24 | OMT24 | Protein | 429 | All |
| SEQ ID NO. 25 | OMT25 | Protein | 435 | All |
| SEQ ID NO. 26 | OMT26 | Protein | 395 | All |
| SEQ ID NO. 27 | OMT27 | Protein | 422 | All |
| SEQ ID NO. 28 | OMT28 | Protein | 423 | All |
| SEQ ID NO. 29 | OMT29 | Protein | 374 | All |
| SEQ ID NO. 30 | OMT30 | Protein | 224 | All |
| SEQ ID NO. 31 | GLY1 | Protein | 458 | All |
| SEQ ID NO. 32 | GLY2 | Protein | 462 | All |
| SEQ ID NO. 33 | GLY3 | Protein | 340 | All |
| SEQ ID NO. 34 | GLY4 | Protein | 470 | All |
| SEQ ID NO. 35 | GLY5 | Protein | 482 | All |
| SEQ ID NO. 36 | GLY6 | Protein | 478 | All |
| SEQ ID NO. 37 | GLY7 | Protein | 479 | All |
| SEQ ID NO. 38 | GLY8 | Protein | 458 | All |
| SEQ ID NO. 39 | GLY9 | Protein | 485 | All |
| SEQ ID NO. 40 | GLY10 | Protein | 485 | All |
| SEQ ID NO. 41 | GLY11 | Protein | 496 | All |
| SEQ ID NO. 42 | HAL1 | Protein | 420 | All |
| SEQ ID NO. 43 | HAL2 | Protein | 333 | All |
| SEQ ID NO. 44 | HAL3 | Protein | 519 | All |
| SEQ ID NO. 45 | HAL4 | Protein | 530 | All |
| SEQ ID NO. 46 | HAL5 | Protein | 530 | All |
| SEQ ID NO. 47 | HAL6 | Protein | 409 | All |
| SEQ ID NO. 48 | HAL7 | Protein | 518 | All |
| SEQ ID NO. 49 | HAL8 | Protein | 425 | All |
| SEQ ID NO. 50 | HAL9 | Protein | 413 | All |
| SEQ ID NO. 51 | HAL10 | Protein | 520 | All |
| SEQ ID NO. 52 | HAL11 | Protein | 528 | All |
| SEQ ID NO. 53 | HAL12 | Protein | 534 | All |
| SEQ ID NO. 54 | HAL13 | Protein | 528 | All |
| SEQ ID NO. 55 | HAL14 | Protein | 409 | All |
| SEQ ID NO. 56 | HAL15 | Protein | 425 | All |
| SEQ ID NO. 57 | HAL16 | Protein | 417 | All |
| SEQ ID NO. 58 | HAL17 | Protein | 517 | All |
| SEQ ID NO. 59 | HAL18 | Protein | 413 | All |
| SEQ ID NO. 60 | HAL19 | Protein | 519 | All |
| SEQ ID NO. 61 | HAL20 | Protein | 518 | All |
| SEQ ID NO. 62 | RFP | Protein | 233 | All |
| SEQ ID NO. 63 | PLAS-400 | DNA | 6484 | 2890-3588 |
| SEQ ID NO. 64 | PLAS-516 | DNA | 6319 | 2890-3423 |
| SEQ ID NO. 65 | PLAS-517 | DNA | 6955 | 2890-4059 |
| SEQ ID NO. 66 | PLAS-518 | DNA | 6886 | 2890-3990 |
| SEQ ID NO. 67 | PLAS-519 | DNA | 6727 | 2890-3831 |
| SEQ ID NO. 68 | PLAS-520 | DNA | 6883 | 2890-3912 |
| SEQ ID NO. 69 | PLAS-521 | DNA | 6809 | 2890-3912 |
| SEQ ID NO. 70 | PLAS-522 | DNA | 7225 | 2890-4329 |
| SEQ ID NO. 71 | PLAS-523 | DNA | 7345 | 2890-4449 |
| SEQ ID NO. 72 | PLAS-524 | DNA | 7399 | 2890-4443 |
| SEQ ID NO. 73 | NpgA | DNA | 3564 | 1170-2201 |
| SEQ ID NO. 74 | DiPKS^{G1516R}-1 | DNA | 11114 | 849-10292 |
| SEQ ID NO. 75 | DIPKS^{G1516R}-2 | DNA | 10890 | 717-10160 |
| SEQ ID NO. 76 | DiPKS^{G1516R}-3 | DNA | 11300 | 795-10238 |
| SEQ ID NO. 77 | DiPKS^{G1516R}-4 | DNA | 11140 | 794-10237 |
| SEQ ID NO. 78 | DiPKS^{G1516R}-5 | DNA | 11637 | 1172-10615 |
| SEQ ID NO. 79 | PDH | DNA | 7114 | Ald6: 1444-2949 |
| | | | | ACS: 3888-5843 |
| SEQ ID NO. 80 | Maf1 | DNA | 3256 | 936-2123 |
| SEQ ID NO. 81 | Erg20K197E | DNA | 4254 | 2842-3900 |
| SEQ ID NO. 82 | Erg1p:UB14-Erg20:deg | DNA | 3503 | 1364-2701 |
| SEQ ID NO. 83 | tHMGr-IDI | DNA | 4843 | tHMGR1: 885-2393 |
| | | | | IDI1: 3209-4075 |
| SEQ ID NO. 84 | PGK1p:ACC1^{S659A,S1157A} | DNA | 7673 | Pgk1p: 222-971 |
| | | | | Acc1mut: 972-7673 |
| SEQ ID NO. 85 | OAC | DNA | 2177 | 842-1150 |
| SEQ ID NO. 86 | PT254 | DNA | 3600 | 1443-2414 |
| SEQ ID NO. 87 | Ostl-pro-alpha- | DNA | 4684 | 1505-3355 |
| | f(1)-OXC53 | | | |
| SEQ ID NO. 88 | NPGA | Protein | 344 | All |
| SEQ ID NO. 89 | DiPKS^{G1516R} | Protein | 3147 | All |
| SEQ ID NO. 90 | OAC | Protein | 102 | All |
| SEQ ID NO. 91 | PT254 | Protein | 323 | All |
| SEQ ID NO. 92 | Ostl-pro-alpha-f(1)-OXC53 | Protein | 610 | All |

The protein encoded by the nucleotide sequence with which the host cell is transformed may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the subject sequence.

The nucleotide sequence may have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the subject sequence.

Expression vectors comprising a subject nucleotide sequence encoding a subject protein are described. In such an expression vectors, the nucleotide sequence encoding the subject protein may comprise, for example, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the relevant residue positions of the subject sequence.

A host cell is described herein that is transformed with any one of the expression vectors described, wherein transformation occurs according to any known process.

The host cell may be a bacterial cell, a fungal cell, a protist cell, or a plant cell, such as any cell described herein.

Non limiting examples of phytocannabinoids that may be used, and their acids, include tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM). Acid forms of phytocannabinoids include cannabigorcinic acid (CBGOa) tetrahydrocannabinolic acid (THCa), cannabidiolic acid (CBDa), cannabichromenic acid (CBCa), cannabigerolic acid (CBGa), cannabigerovarinic acid (CBGVa) and tetrahydrocannabivarin acid (THCVa). Any such cannabinoid or phytocannabinoid acid can be utilized in the process according to the invention.

In some examples described herein, the polyketide or cannabinoid precursor may be olivetol, olivetolic acid, divarin, divarinic acid, orcinol, or orsellinic acid. Methods by which polyketides can be converted into phytocannabinoids are described in Applicant's co-pending PCT Patent Appln No. PCT/CA2020/050687 (Bourgeois et al.) entitled METHODS AND CELLS FOR PRODUCTION OF PHYTOCANNABINOIDS AND PHYTOCANNABINOID PRECURSORS filed May 21, 2020, the entirety of which is hereby incorporated by reference. For example, the following precursors may be converted to the following phytocannabinoid, and may be O-methylated, glycosylated or chlorinated, as described herein: olivetol can be used to form cannabigerol (CBG), olivetolic acid can be used to form cannabigerolic acid (CBGa), divarin can be used to form cannabigerovarin (CBGv), divarinic acid can be used to form cannabigerovarinic acid (CBGva), orcinol can be used to form cannabigerocin (CBGo), and orsellinic acid can be used to form cannabigerocinic acid (CBGoa).

To gain a better understanding of the invention described herein, the following examples are set forth. It should be understood that these examples are for illustrative purposes only. Therefore, they should not limit the scope of this invention in anyway.

### EXAMPLES

In the following Examples, certain aspects of materials and methods are shared, and thus are described generally hereinbelow.

### Strain Growth and Media

In general, yeast strains are grown on yeast minimal media with a composition of 1.7 g/L YNB without ammonium sulfate+1.96 g/L URA dropout amino acid supplements+1.5 g/L magnesium L-glutamate; and with 2% w/v galactose, 2% w/v raffinose, 200 μg/l geneticin, and 200 ug/L ampicillin (Sigma-Aldrich Canada), optionally with other ingredients and variations where indicated below, for 96 hours. HB2130 expresses a non-catalytic mScarlett protein and serves as a negative control.

### Experimental Conditions

Unless otherwise indicated, 3 colony replicates of strains were tested in the examples described. Strains are grown in 1 ml media for 96 hours in 96-well deepwell plates. The deepwell plates were incubated at 30° C. and shaken at 950 rpm for 96 hrs. Metabolite extraction was performed by adding 270 μl of 56% acetonitrile to 30 μl of culture in a fresh 96-well deepwell plates. The plates were then centrifuged at 3750 rpm for 5 min. 200 μl of the soluble layer was removed and stored in a 96-well v-bottom microtiter plate. Samples were stored at −20° C. until analysis.

### Quantification Protocol

LC Conditions. Quantification of the cannabinoid of interest in the examples, such as O-methyl THCa, glycosylated CBGa, and chlorinated olivetolic acid, was done using an Agilent™ 6560 ion mobility-QTOF. The chromatography and MS conditions are described below.

Column: Acquity UPLC BEH C18 1.7 micron 2.1×5 mm

Column temperature: 45° C.

Flow rate: 0.3 ml/min

Eluent A: Water 100%

Eluent B: Acetonitrile 100%

Exact masses are as follows in Table 3, which shows monoisotopic masses of certain analyzed minor cannabinoids and polyketide precursors thereof. Gradient is listed in Table 4.

TABLE 3
Monoisotopic Masses of Cannabinoids or Precursors

| Molecule | M/z Observed |
|---|---|
| O-methyl THCa | 372.2298 |
| Glycosyl-CBGa | 539.2833 |
| Chlorinated Olivetolic Acid | 259.0737 |

TABLE 4
Column Gradient (% Eluent B)

| Time (min) | % B |
|---|---|
| 0.00 | 30 |
| 3.50 | 95 |
| 3.60 | 95 |
| 4.60 | 95 |
| 4.70 | 30 |
| 7.00 | 30 |

ESI-MS conditions. The ESI-MS conditions were as follows:

Capillary: 3.5 kV

Source temperature: 150° C.

Desolvation gas temperature: 300° C.

Drying gas flow (nitrogen): 600 L/hr

Sheath gas flow (nitrogen): 660 L/hr

Table 5 outlines the plasmids used herein.

TABLE 5
Plasmids Used

| # | Plasmid Name | Description | Selection | Backbone |
|---|---|---|---|---|
| 1 | PLAS-400 | Gal1p:mScarlett:Cyc1t | Uracil | pYES-URA |
| 2 | PLAS-516 | Gal1p:OMT7:Cyc1t | Uracil | pYES-URA |
| 3 | PLAS-517 | Gal1p: OMT8:Cyc1t | Uracil | pYES-URA |
| 4 | PLAS-518 | Gal1p:OMT10:Cyc1t | Uracil | pYES-URA |
| 5 | PLAS-519 | Gal1p:OMT11:Cyc1t | Uracil | pYES-URA |
| 6 | PLAS-520 | Gal1p:OMT12:Cyc1t | Uracil | pYES-URA |
| 7 | PLAS-521 | Gal1p:GLY3:Cyc1t | Uracil | pYES-URA |
| 8 | PLAS-522 | Gal1p:GLY7:Cyc1t | Uracil | pYES-URA |
| 9 | PLAS-523 | Gal1p:HAL19:Cyc1t | Uracil | pYES-URA |
| 10 | PLAS-524 | Gal1p:HAL20:Cyc1t | Uracil | pYES-URA |

Table 6 outlines the yeast strains used In the following Examples.

TABLE 6
Yeast Strains

| Strain # | Background | Plasmids | Genotype | Notes |
|---|---|---|---|---|
| HB42 | -URA, -LEU | None | Saccharomyces cerevisiae | Base strain |
| | | | CEN.PK2; ΔLEU2; ΔURA3; | |
| | | | Erg20K197E::KanMx | |
| HB1254 | -URA, -LEU | | Saccharomyces cerevisiae | Base THCa |
| | | | CEN.PK2; ΔLEU2; ΔURA3; | producing strain |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |
| HB2031 | -URA, -LEU | PLAS-400 | Saccharomyces cerevisiae | Negative Control |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(I)-OXC53 | |
| HB2032 | -URA, -LEU | PLAS-516 | Saccharomyces cerevisiae | Expresses OMT7 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DIPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |
| HB2033 | -URA, -LEU | PLAS-517 | Saccharomyces cerevisiae | Expresses OMT8 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(I)-OXC53 | |
| HB2034 | -URA, -LEU | PLAS-518 | Saccharomyces cerevisiae | Expresses OMT10 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(I)-OXC53 | |
| HB2035 | -URA, -LEU | PLAS-519 | Saccharomyces cerevisiae | Expresses OMT11 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(I)-OXC53 | |
| HB2036 | -URA, -LEU | PLAS-520 | Saccharomyces cerevisiae | Expresses OMT12 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |
| HB2037 | -URA, -LEU | PLAS-521 | Saccharomyces cerevisiae | Expresses Gly3 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |
| HB2038 | -URA, -LEU | PLAS-522 | Saccharomyces cerevisiae | Expresses Gly7 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |
| HB2039 | -URA, -LEU | PLAS-523 | Saccharomyces cerevisiae | Expresses HAL19 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |
| HB2040 | -URA, -LEU | PLAS-524 | Saccharomyces cerevisiae | Expresses HAL20 |
| | | | CEN.PK2; ΔLEU2; ΔURA3; | |
| | | | Erg20K197E::KanMx; ALD6; | |
| | | | ASC1L641P; NPGA; MAF1; | |
| | | | PGK1p:Acc1; tHMGR1; | |
| | | | IDI; DiPKS_G1516R X5; | |
| | | | ACC1_S659A_S1157A; | |
| | | | UB14p:ERG20; pGAL:OAC; | |
| | | | pGAL:PT254; Ostl-pro- | |
| | | | alpha-f(l)-OXC53 | |

Table 7 lists modifications that may be incorporated into base strains used in the following Examples.

TABLE 7
Modifications to Base Strains

| | | Integration | | |
|---|---|---|---|---|
| Modification | SEQ ID | Region/ | | Genetic Structure |
| Name | NO. | Plasmid | Description | of Sequence |
| 1. | SEQ ID | Flagfeldt Site | Phosphopantetheinyl | Site14Up::Tef1p:NpgA:Prm9t: |
| NpgA | NO. 73 | 14 | Transferase from Aspergillus | Site14Down |
| | | integration | niger. Accessory Protein for | |
| | | | DIPKS | |
| 2. | SEQ ID | USER Site | Type 1 FAS fused to Type 3 | XII- |
| DIPKS^{G1516R}-1 | NO.74 | XII-1 | PKS from D. discoideum. | 1up::Gal1p:DiPKSG1516R: |
| | | integration | Produces Olivetol from | Prm9t::XII1-down |
| | | | malonyl-coA | |
| 3. | SEQ ID | Wu site 1 | Type 1 FAS fused to Type 3 | Wu1up::Gal1p:DiPKSG1516R: |
| DIPKS^{G1516R}-2 | NO. 75 | integration | PKS from D. discoideum. | Prm9t::Wu1down |
| | | | Produces Olivetol from | |
| | | | malonyl-coA | |
| 4. | SEQ ID | Wu site 3 | Type 1 FAS fused to Type 3 | Wu3up::Gal1p:DiPKSG1516R: |
| DIPKS^{G1516R}-3 | NO. 76 | integration | PKS from D. discoideum. | Prm9t::Wu3down |
| | | | Produces Olivetol from | |
| | | | malonyl-coA | |
| 5. | SEQ ID | Wu site 6 | Type 1 FAS fused to Type 3 | Wu6up::Gal1p:DiPKSG1516R: |
| DIPKS^{G1516R}-4 | NO. 77 | integration | PKS from D. discoideum. | Prm9t::Wu6down |
| | | | Produces Olivetol from | |
| | | | malonyl-coA | |
| 6 | SEQ ID | Wu site 18 | Type 1 FAS fused to Type 3 | Wu18up::Gal1p:DiPKSG1516R: |
| DIPKS^{G1516R}-5 | NO. 78 | integration | PKS from D. discoideum. | Prm9t::Wu18down |
| | | | Produces Olivetol from | |
| | | | malonyl-coA | |
| 7. | SEQ ID | Flagfeldt Site | Acetaldehyde dehydrogenase | 19Up::Tdh3p:Ald6:Adh1:: |
| PDH | NO. 79 | 19 | (ALD6) from S. cerevisiae and | Tef1p:seACS1^{L641P}:Prm9t:: |
| | | integration | acetoacetyl coA synthase | 19Down |
| | | | (AscL641P) from Salmonella | |
| | | | enterica. Will allow greater | |
| | | | accumulation of acetyl-coA in | |
| | | | the cell. | |
| 8. | SEQ ID | Flagfeldt Site | Maf1 is a regulator of tRNA | Site5Up::Tef1p:Maf1:Prm9t: |
| Maf1 | NO. 80 | 5 integration | biosynthesis. Overexpression | Site5Down |
| | | | in S. cerevisiae has | |
| | | | demonstrated higher | |
| | | | monoterpene (GPP) yields | |
| 9. | SEQ ID | Chromosomal | Mutant of Erg20 protein that | Tpi1t:ERG20K197E:Cyc1t:: |
| Erg20K197E | NO. 81 | modification | diminishes FPP synthase | Tef1p:KanMX:Tef1t |
| | | | activity creating greater pool | |
| | | | of GPP precursor. Negatively | |
| | | | affects growth phenotype. | |
| 10. | SEQ ID | Flagfeldt Site | Sterol responsive promoter | Site18Up::Erg1p:UB14deg: |
| Erg1p:UB14- | NO. 82 | 18 | controlling Erg20 protein | ERG20:Adh1t:Site18down |
| Erg20:deg | | integration | activity. Allows for regular | |
| | | | FPP synthase activity and | |
| | | | uninhibited growth phenotype | |
| | | | until accumulation of sterols | |
| | | | which leads to a suppression | |
| | | | of expression of enzyme. | |
| 11. | SEQ ID | USER Site | Overexpression of truncated | X3up::Tdh3p:tHMGR1:Adh1t:: |
| tHMGr-IDI | NO. 83 | X-3 | HMGr1 and IDI1 proteins that | Tef1p:IDI1:Prm9t::X3down |
| | | integration | have been previously | |
| | | | identified to be bottlenecks in | |
| | | | the S. cerevisiae terpenoid | |
| | | | pathway responsible for GPP | |
| | | | production. | |
| 12. | SEQ ID | Chromosomal | Mutations in the native S. | Pgk1:ACC1^{S659A,S1157A}:Acc1t |
| PGK1p:ACC1^{S659A,S1157A} | NO. 84 | modification | cerevisiae acetyl-coA | |
| | | | carboxylase that removes | |
| | | | post-translational modification | |
| | | | based down-regulation. Leads | |
| | | | to greater malonyl-coA pools. | |
| | | | The promoter of Acc1 was | |
| | | | also changed to a constitutive | |
| | | | promoter for higher | |
| | | | expression. | |
| 13. | SEQ ID | Flagfeldt Site | The Cannabis sativa Olivetolic | FgF16up::Gal1p:csOAC: |
| OAC | NO. 85 | 16 | acid cyclase (OAC) protein | Eno2t::FgF16down |
| | | integration | allows the production of | |
| | | | olivetolic acid from a | |
| | | | polyketide precursor. . | |
| 14. | SEQ ID | Flagfeldt Site | PT254 is a truncated | FgF20up::Gal1p:PT254:Cyct:: |
| PT254 | NO. 86 | 20 | cannabigerolic acid synthase | FgF20down |
| | | integration | from C. sativa | |
| 15. | SEQ ID | Apel-3 | d28 THC synthase fused with | Apel-3up::Tef1p:Ostl-pro- |
| Ostl-pro- | NO. 87 | | a 5' Ostl-pro-alpha-f(l) tag | alpha-f(l)- |
| alpha-f(l)- | | | | OXC53::cyct:Apel-3down |
| OXC53 | | | | |

### EXAMPLE 1

Production of O-methylated THCa

Introduction. Phytocannabinoids are naturally produced in Cannabis sativa, other plants, and some fungi. Over 105 phytocannabinoids are known to be biosynthesized in C. sativa, or result from thermal or other decomposition from phytocannabinoids biosynthesized in C. sativa. While the C. sativa plant is also a valuable source of grain, fiber, and other material, growing C. sativa for phytocannabinoid production, particularly indoors, is costly in terms of energy and labour. Subsequent extraction, purification, and fractionation of phytocannabinoids from the C. sativa plant is also labour and energy intensive.

Production of phytocannabinoids in genetically modified strains of Saccharomyces cerevisiae is conducted. In particular, A THCa producing yeast strain (HB1254) was transformed with a plasmid expressing either RFP or an enzyme selected from OMT1-OMT30. In order to produce O-methyl THCa, strains HB2031 to HB2036 were grown on yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin (Sigma-Aldrich Canada) for 96 hours. Under these conditions the strains produced THCa and these molecules are O-methylated due to the presence of appropriate enzymes. HB2031, expressing a non-catalytic mScarlett protein, was used as a negative control.

Following growth and MS analysis, O-methyl THCa (Formula I) was detected in some samples. No other O-methylated cannabinoids or cannabinoid precursors were observed. Quantities of O-methyl THCa produced by these strains are summarized in Table 8). The values reported are the average of 3 biological replicates.

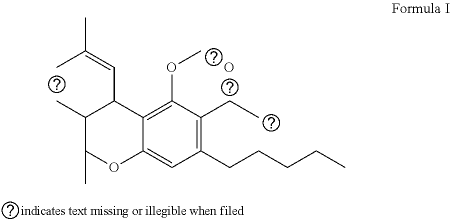

TABLE 8
Quantities of O-methyl THCa Produced by Modified Yeast Strains

| Strain | Plasmid | Enzyme | O-methyl THCA (AU) |
|---|---|---|---|
| HB2031 | PLAS-400 | None (RFP) | 0 |
| HB2032 | PLAS-516 | OMT7 | 34569.64 |
| HB2033 | PLAS-517 | OMT8 | 86374.55 |
| HB2034 | PLAS-518 | OMT10 | 6291.63 |
| HB2035 | PLAS-519 | OMT11 | 325438.45 |
| HB2036 | PLAS-520 | OMT12 | 87847.46 |

### EXAMPLE 2

### Production of Glycosylated CBGa

Production of phytocannabinoids in genetically modified strains of Saccharomyces cerevisiae is described. In particular, a THCa producing yeast strain (HB1254) was transformed with a plasmid expressing either RFP or an enzyme selected from GLY1-11.

Production of glycosylated CBGa in the resulting strains HB2037-HB2038 was conducted by growth on yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin (Sigma-Aldrich Canada) for 96 hours. Under these conditions the strains produce CBGa, which is then glycosylated by the appropriate enzymes present. HB2031, which expresses a non-catalytic mScarlett protein, and served as a negative control.

Following growth and MS analysis glycosylated CBGa was detected in some samples. No other glycosylated cannabinoids or cannabinoid precursors were observed. Quantities of glycosylated CBGa produced by these strains are summarized in Table 9). The CBGa was found to be mono-glycosylated, although from this analysis we could not determine which alcohol residue is the attachment site for the glycosyl residue. The values reported are the average of 3 biological replicates. Two possible structures of monoglycosylated CBGa are provided as Formula II and III.

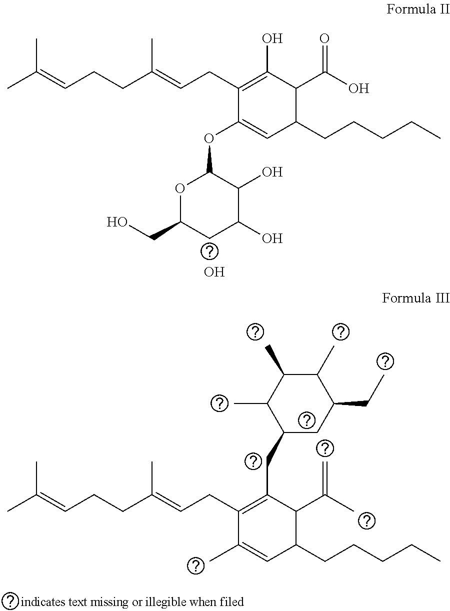

TABLE 9
Quantities of Glycosylated CBGa Produced by Modified Yeast Strains

| Strain | Plasmid | Enzyme | Glycosyl CBGa (AU) |
|---|---|---|---|
| HB2031 | PLAS-400 | None (RFP) | 0 |
| HB2037 | PLAS-521 | Glu 3 | 270689.0885 |
| HB2038 | PLAS-522 | Glu 7 | 195152.1875 |

### EXAMPLE 3

### Production of Production of Chlorinated Olivetolic Acid

Production of phytocannabinoids in genetically modified strains of Saccharomyces cerevisiae is described. In particular, a THCa producing yeast strain (HB1254) was transformed with a plasmid expressing either RFP or an enzyme selected from HAL1-HAL20.

In order to produce chlorinated olivetolic acid, HB2030, HB2039, HB2040 were grown on yeast minimal media with a composition of 1.7 g/L YNB without ammonium sulfate+1.96 g/L URA dropout amino acid supplements+1.5 g/L magnesium L-glutamate) with 2% w/v galactose, 2% w/v raffinose, 200 μg/l geneticin, and 200 ug/L ampicillin (Sigma-Aldrich Canada)+100 mg/L sodium chloride+100 mg/L+100 mg/L potassium bromide+100 mg/L sodium iodide for 96 hours. The strains produced olivetolic acid, and the molecule was then halogenated with a chlorine, bromine or iodine, with the appropriate halogenase being present. HB2031 was used as a negative control, as it expresses a non-catalytic mScarlett protein.

Strains were grown in media containing chlorine, bromine and iodine salts. After MS analysis chlorinated olivetolic acid was detected in some samples. No other halogenated (Br, I, Cl) cannabinoids or cannabinoid precursors were found. The quantities of chlorinated olivetolic acid produced by these strains is summarized in Table 10. The values reported are the average of 3 biological replicates. While no halogenated cannabinoids were observed in this experiment, the biosynthesis of halogenated cannabinoids either from the precursors formed as described, or via a separate pathway can be achieved in the presence of the appropriate prenyltransferase.

Formula IV shows structure of halogenated olivetolic acid.

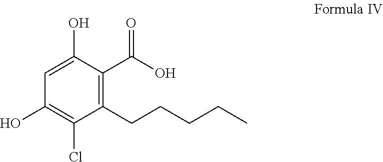 [SMILES: CCCCCc1c(Cl)c(O)cc(O)c1C(=O)O]

TABLE 10
Quantities of Halogenated Olivetolic Acid
Produced by Modified Yeast Strains

| Strain | Plasmid | Halogenase | Chlorinated Olivetolic acid (AU) |
|---|---|---|---|
| HB2031 | PLAS-400 | None (RFP) | 0 |
| HB2039 | PLAS-523 | Hal19 | 415436.67 |
| HB2040 | PLAS-524 | Hal20 | 150236.51 |

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

### REFERENCES

All publications, patents and patent applications mentioned in this specification are indicative of the level of skill those skilled in the art to which this invention pertains and are herein incorporated by reference to the same extent as if each individual publication, patent, or patent application was specifically and individually indicated to be incorporated by reference.

### Patent Publications

U.S. Pat. No. 7,361,482

U.S. Pat. No. 8,884,100 (Page et al.) Aromatic Prenyltransferase from Cannabis.

U.S. Pat. No. 9,066,920 (Whalley et al.)

U.S. Patent Publication No. US2019/0338301A1 (Sayre, R. T., Goncalves, E. C., & Zidenga, T.) High level in vivo biosynthesis and isolation of watersoluble cannabinoids in stably transformed plant systems.

WO2017/161041 A1 (Gonazlez, R., et al.) MICROBIAL SYNTHESIS OF ISOPRENOID PRECURSORS, ISOPRENOIDS AND DERIVATIVES INCLUDING PRENYLATED AROMATICS COMPOUNDS, published Sep. 21, 2017

WO2018/148848 A1 (Mookerjee et al.) publication of PCT/CA2018/050189, METHOD AND CELL LINE FOR PRODUCTION OF PHYTOCANNABINOIDS AND PHYTOCANNABINOID ANALOGUES IN YEAST

WO2018/148849 (Mookerjee et al.) publication of PCT/CA2018/050190, METHOD AND CELL LINE FOR PRODUCTION OF POLYKETIDES IN YEAST.

PCT Patent Appln No. PCT/CA2020/050687 (Bourgeois et al.) METHODS AND CELLS FOR PRODUCTION OF PHYTOCANNABINOIDS AND PHYTOCANNABINOID PRECURSORS filed May 21, 2020.

### Non-Patent Literature

Bai Flagfeldt, D., Siewers, V., Huang, L. and Nielsen, J. (2009) “Characterization of chromosomal integration sites for heterologous gene expression in Saccharomyces cerevisiae” Yeast, 26, 545-551.

Caprioglio, D., Allegrone, G., Pollastro, F., Valera, S., Lopatriello, A., Collado, J. A., . . . & Taglialatela-Scafati, O. (2019). O-Methyl Phytocannabinoids: Semi-synthesis, Analysis in Cannabis Flowerheads, and Biological Activity. Planta medica, 85 (11/12), 981-986.

Garcia, C., Palomo-Garo, C., Garcia-Arencibia, M., Ramos, J. A., Pertwee, R. G., & Fernández-Ruiz, J. (2011). Symptom-relieving and neuroprotective effects of the phytocannabinoid Δ9-THCV in animal models of Parkinson's disease. British journal of pharmacology, 163 (7), 1495-1506.

Gagne, S. J., et al. (2012) “Identification of Olivetolic Acid Cyclase from Cannabis sativa Reveals a Unique Catalytic Route to Plant Polyketides.” Proceedings of the National Academy of Sciences, vol. 109, no. 31, 2012, pp. 12811-12816. doi:10.1073/pnas.1200330109.

GenBank: LC381857.1, Rhododendron dauricum Rd-1 RDPT1 mRNA for orsellinic acid 3-farnesyltransferase, 1475 bp, submitted 9 Apr. 2018 (19 Apr. 2018). Retrieved from: www.ncbl.nlm.nih.aov/nuccQre/LC381857

Ghosh, R., A. Chhabra, P. A. Phatale, S. K. Samrat, J. Sharma, A. Gosain, D. Mohanty, S. Saran and R. S. Gokhale (2008) “Dissecting the Functional Role of Polyketide Synthases in Dictyostelium discoideum biosynthesis of the differentiation regulating factor 4-methyl-5-pentylbenzene-1,3-diol” Journal of Biological Chemistry, 283 (17), 11348-11354.

Gietz, R. D. and Schiestl, R. H., (2007) “High-efficiency yeast transformation using the LiAc/SS carrier DNA/PEG method.” Nat. Protoc. 2, 31-34.

Gietz R. D. (2014) Yeast Transformation by the LiAc/SS Carrier DNA/PEG Method (pp 1-12). In: Smith J., Burke D. (eds) Yeast Genetics. Methods in Molecular Biology (Methods and Protocols), vol 1205. Humana Press, New York, N.Y. https://doi.org/10.1007/978-1-4939-1363-3_1.

Hardman, Janee'M, H., Brooke, R. T., & Zipp, B. J. (2017). Cannabinoid glycosides: In vitro production of a new class of cannabinoids with improved physicochemical properties. BioRxiv, 104349.

Jensen, N. B., Strucko, T., Kildegaard, K. R., David, F., Er\hat{ }Ome Maury, J., Mortensen, U. H., et al., (2014). EasyClone: method for iterative chromosomal integration of multiple genes in Saccharomyces cerevisiae. FEMS Yeast Research, Volume 14, Issue 2, pages 238-248; https://doi.org/10.1111/1567-1364.12118.

Kim, J.-M., Song, H.-Y., Choi, H.-J., So, K.-K., Kim, D.-H., Chae, K.-S., . . . Jahng, K.-Y. (2015). “Characterization of NpgA, a 4′-phosphopantetheinyl transferase of Aspergillus nidulans, and evidence of its involvement in fungal growth and formation of conidia and cleistothecia for development.” Journal of Microbiology, 53 (1), 21-31 https://doi.org/10.1007/s12275-015-4657-8.

Kuzuyama et al. (2005) Structural basis for the promiscuous biosynthetic prenylation of aromatic natural products, Nature, volume 435, pages 983-987; doi: 10.1038/nature03668.

Liu, J., Zhang, W., Du, G., Chen, J., & Zhou, J. (2013). “Overproduction of geraniol by enhanced precursor supply in Saccharomyces cerevisiae.” Journal of Biotechnology, 168 (4), 446-451. https://doi.org/10.1016/J.JBIOTEC.2013.10.017.

Luo, X., Reiter, M., d'Espaux, L., Wong, J., Denby, C., Lechner, A., Zhang, Y., Grzybowski, A., Harth, S., Lin, W., Lee, H., Yu, C., Shin, J., Deng, K., Benites, V., Wang, G., Baidoo, E., Chen, Y., Dev, I., Petzold, C. and Keasling, J. (2019). “Complete biosynthesis of cannabinoids and their unnatural analogues in yeast.” Nature, 567 (7746), pp.123-126.

Okada, M., Saito, K., Wong, C. P., Li, C., Wang, D., Iijima, M., . . . & Abe, I. (2017). Combinatorial biosynthesis of (+)-daurichromenic acid and its halogenated analogue. Organic letters, 19 (12), 3183-3186.

Oswald, Marilyne; Marc Fischer, Nicole Dirninger, Francis Karst, (2007) “Monoterpenoid biosynthesis in Saccharomyces cerevisiae.” FEMS Yeast Research, 7 (3), 413-421. https://doi.org/10.1111/j.1567-1364.2006.00172.x

Peng, B., Nielsen, L. K., Kampranis, S. C., & Vickers, C. E. (2018). Engineered protein degradation of farnesyl pyrophosphate synthase is an effective regulatory mechanism to increase monoterpene production in Saccharomyces cerevisiae. Metabolic Engineering, 47, 83-93. https://doi.org/10.1016/J.YMBEN.2018.02.005.

Ro, D.-K., Paradise, E. M., Ouellet, M., Fisher, K. J., Newman, K. L., Ndungu, J. M., Keasling, J. D. (2006). Production of the antimalarial drug precursor artemisinic acid in engineered yeast. Nature, 440 (7086), 940-943. JOUR. https://doi.org/10.1038/nature04640.

Ryan, O. W., Poddar, S., & Cate, J. H. D. (2016). CRISPR-Cas9 Genome Engineering in Saccharomyces cerevisiae Cells. Cold Spring Harbor Protocols, 2016 (6), pdb.prot086827. https://doi.org/10.1101/pdb.prot086827.

Saeki, H., Hara, R., Takahashi, H., Iijima, M., Munakata, R., Kenmoku, H., . . . Taura, F. (2018). An Aromatic Farnesyltransferase Functions in Biosynthesis of the Anti-HIV Meroterpenoid Daurichromenic Acid. Plant Physiology, 178 (2), 535-551; https://doi.org/10.1104/PP.18.00655.

Shi, S., Chen, Y., Siewers, V., & Nielsen, J. (2014). “Improving Production of Malonyl Coenzyme A-Derived Metabolites by Abolishing Snf1-Dependent Regulation of Acc1.” mBio, 5 (3), e01130-14. https://doi.org/10.1128/mBio.01130-14.

Shiba, Y., Paradise, E. M., Kirby, J., Ro, D.-K., & Keasling, J. D. (2007). “Engineering of the pyruvate dehydrogenase bypass in Saccharomyces cerevisiae for high-level production of isoprenoids.” Metabolic Engineering, 9 (2), 160-168. https://doi.org/10.1016/J.YMBEN.2006.10.005.

Stout, J. M., Boubakir, Z., Ambrose, S. J., Purves, R. W., & Page, J. E. (2012). The hexanoyl-CoA precursor for cannabinoid biosynthesis is formed by an acyl-activating enzyme in Cannabis sativa trichomes. The Plant Journal, 71 (3), 353-365.

Takeda, S., Usami, N., Yamamoto, I., & Watanabe, K. (2009). Cannabidiol-2′, 6′-dimethyl ether, a cannabidiol derivative, is a highly potent and selective 15-lipoxygenase inhibitor. Drug metabolism and disposition, 37 (8), 1733-1737.

Taura, Futoshi, et al. (2009) “Characterization of olivetol synthase, a polyketide synthase putatively involved in cannabinoid biosynthetic pathway.” FEBS Letters Vol. 583, 12 (2009): 2061-2066.

Usami, N., Okuda, T., Yoshida, H., Kimura, T., Watanabe, K., Yoshimura, H., & Yamamoto, I. (1999). Synthesis and pharmacological evaluation in mice of halogenated cannabidiol derivatives. Chemical and pharmaceutical bulletin, 47 (11), 1641-1645.

Varshaysky, A. (2011). The N-end rule pathway and regulation by proteolysis. Protein Science 20 (8):1285-1476. https://doi.org/10.1002/pro.666.

Wargent, E. T., Zaibi, M. S., Silvestri, C., Hislop, D. C., Stocker, C. J., Stott, C. G., . . . & Cawthorne, M. A. (2013). The cannabinoid Δ9-tetrahydrocannabivarin (THCV) ameliorates insulin sensitivity in two mouse models of obesity. Nutrition & diabetes, 3 (5), e68-e68.

YAMAUCHI, T., SHOYAMA, Y., MATSUO, Y., & NISHIOKA, I. (1968). Cannabigerol monomethyl ether, a new component of hemp. Chemical and Pharmaceutical Bulletin, 16 (6), 1164-1165.

1. A method of producing a substituted phytocannabinoid or a substituted phytocannabinoid precursor in a host cell that produces the phytocannabinoid or the phytocannabinoid precursor, said method comprising:
transforming said host cell with a sequence encoding an enzyme for derivatizing the phytocannabinoid or the phytocannabinoid precursor with the substituent, and
culturing said transformed host cell to produce said substituted phytocannabinoid or said substituted phytocannabinoid precursor,
wherein:
said derivatizing comprises:
(i) O-methylation, wherein the substituent is O-methyl, and the enzyme for derivatizing comprises an O-methylation enzyme selected from the group consisting of an OMT1-OMT30 protein comprising an amino acid sequence of at least 95% identity to the sequence as set forth in any one of SEQ ID NO:1-SEQ ID NO:30;
(ii) glycosylation, wherein the substituent is glycosyl, and the enzyme for derivatizing comprises a glycosylation enzyme selected from the group consisting of a GLY1-GLY11 protein comprising an amino acid sequence of at least 95% identity to the sequence as set forth in any one of SEQ ID NO:31-SEQ ID NO:41; or
(iii) halogenation, wherein the substituent is halogen, and the enzyme for derivatizing comprises a halogenation enzyme selected from the group consisting of a HAL1-HAL20 protein comprising an amino acid sequence of at least 95% identity to the sequence as set forth in any one of SEQ ID NO:42-SEQ ID NO:62.

2. The method of claim 1, wherein:
when said phytocannabinoid is THCa and said substituent is O-methyl;
when said phytocannabinoid is CBGa, and said substituent is glycosyl; or
when said phytocannabinoid precursor is olivetolic acid and said substituent is a halogen.

3. (canceled)

4. (canceled)

5. (canceled)

6. (canceled)

7. (canceled)

8. (canceled)

9. (canceled)

10. (canceled)

11. The method of claim 1, wherein said phytocannabinoid is an acid selected from the group consisting of cannabigorcinic acid (CBGOa) tetrahydrocannabinolic acid (THCa), cannabidiolic acid (CBDa), cannabichromenic acid (CBCa), cannabigerolic acid (CBGa), cannabigerovarinic acid (CBGVa), tetrahydrocannabivarin acid (THCVa), tetrahydrocannabiorsellenic acid (THCOa), and cannabidiorsellenic acid.

12. (canceled)

13. (canceled)

14. The method of claim 1, wherein the substituted phytocannabinoid or substituted phytocannabinoid precursor is O-methylated THCa, glycosylated CBGa, or chlorinated olivetolic acid.

15. (canceled)

16. (canceled)

17. (canceled)

18. (canceled)

19. (canceled)

20. The method of claim 1, wherein the host cell additionally comprises a nucleic acid encoding a protein having an amino acid sequence according to any one of SEQ ID NO:88-SEQ ID NO:92.

21. The method of claim 1, wherein said host cell is a bacterial cell, a fungal cell, a protist cell, or a plant cell.

22. (canceled)

23. The method of claim 21, wherein said host cell is S. cerevisiae, E. coli, Yarrowia lipolytica, or Komagataella phaffii.

24. (canceled)

25. The method of claim 1, wherein the host cell additionally comprises at least one genetic modification comprising:
(a) a nucleic acid as set forth in any one of SEQ ID NO:73 to SEQ ID NO:87;
(b) a nucleic acid having at least 90% sequence identity with the nucleotide sequence of (a);
(c) a nucleic acid that hybridizes with the complementary strand of the nucleic acid of (a);
(d) a nucleic acid encoding a polypeptide with the same enzyme activity as the polypeptide encoded by any one of the nucleic acid sequences of (a);
(e) a nucleotide sequence that differs from (a) by one or more nucleotides that are substituted, deleted, and/or inserted; or
(f) a derivative of (a), (b), (c), (d), or (e).

26. The method of claim 25, wherein the at least one genetic modification comprises a nucleic acid having at least 95%, 96%, 97%, 98%, or 99% identity with the nucleic acid as set forth in (a).

27. The method of claim 1, wherein the host cell additionally comprises a nucleic acid encoding a protein having an amino acid sequence with at least 95% sequence identity with any one of SEQ ID NO:88-SEQ ID NO:92.

28. The method of claim 1, wherein said host cell comprises a plasmid with a nucleotide sequence with at least 95% sequence identity with any one of SEQ ID NO:64-SEQ ID NO:72.

29. An expression vector comprising a nucleotide molecule comprising a polynucleotide sequence encoding an enzyme for derivatizing a phytocannabinoid or a phytocannabinoid precursor with the substituent, wherein said nucleotide sequence encodes an enzyme having an amino acid sequence according to any one of SEQ ID NO:1-SEQ ID NO:62 or encoding an enzyme having at least 70% 95% identity thereto.

30. (canceled)

31. (canceled)

32. The expression vector of claim 29, comprising a nucleotide sequence according to any one of SEQ ID NO:64-SEQ ID NO:72.

33. A host cell transformed with the expression vector according to claim 29.

34. The host cell of claim 33, additionally comprising one or more of:
(a) a nucleic acid as set forth in any one of SEQ ID NO: 73 to SEQ ID NO:87;
(b) a nucleic acid having at least 95% sequence identity with the nucleotide sequence of (a);
(c) a nucleic acid that hybridizes with the complementary strand of the nucleic acid of (a);
(d) a nucleic acid encoding a protein with the same enzyme activity as the protein encoded by any one of the nucleic acid sequences of (a);
(e) a nucleic acid that differs from (a) by one or more nucleotides that are substituted, deleted, and/or inserted; or
(f) a derivative of (a), (b), (c), (d), or (e).

35. (canceled)

36. The host cell of claim 33, additionally comprising a nucleotide sequence encoding a protein with an amino acid sequence according to any one of SEQ ID NO:88-SEQ ID NO:92.

37. (canceled)

38. (canceled)

39. A halogenated olivetolic acid produced by the method of claim 2.

40. A chlorinated olivetolic acid produced by the method of claim 14.

41. Chlorinated olivetolic acid.

42. The method of claim 28, wherein:
derivatizing comprises O-methylation;
the O-methylation enzyme comprises an amino acid sequence of at least 95% identity to SEQ ID NO:1; and
the host cell comprises the plasmid with a nucleotide sequence of at least 85% sequence identity with SEQ ID NO:64.
